(19) United States
(12) Patent Application Publication
(10) Pub. No.: US 2009/0233960 A1
(43) Pub. Date: Sep. 17, 2009

(54) Kinase Inhibitors

(75) Inventors: Philippe Van Rompaey, Melle (BE); Philippe Arzel, Gent (BE); Olivier Raynald Defert, Le Havre (FR); Dirk Casimir Maria Leysen, Lokeren (BE)

Correspondence Address: WOLF GREENFIELD & SACKS, P.C., 600 ATLANTIC AVENUE, BOSTON, MA, 02210-2206, US

(73) Assignee: Devgen NV, Zwijnaarde (BE)

(21) Appl. No.: 12/083,408

(22) PCT Filed: Oct. 13, 2006
(86) PCT No.: PCT/EP2006/009923

§ 371 Date: Apr. 11, 2008

Related U.S. Application Data
(60) Provisional application No. 60/726,523, filed on Oct. 13, 2005.

(51) Int. Cl. A61K 31/437; A61K 31/44; C07D 471/04; C07D 213/02

(52) U.S. Cl. 514/303; 514/300; 514/352; 546/113; 546/119; 546/309

(57) ABSTRACT

The present invention relates to new AGC kinase inhibitors, in particular to compounds of Formula I, II, or III or a stereoisomer, tautomer, racemic, metabolite, pro- or predrug, salt, hydrate, or solvate thereof, I II III wherein X, R¹, R², R³, R³¹, n, and m have the meaning defined in the claims. In particular, the present invention relates more specifically to ROCK inhibitors, compositions, in particular pharmaceuticals, comprising such inhibitors, and to uses of such inhibitors in the treatment and prophylaxis of disease.

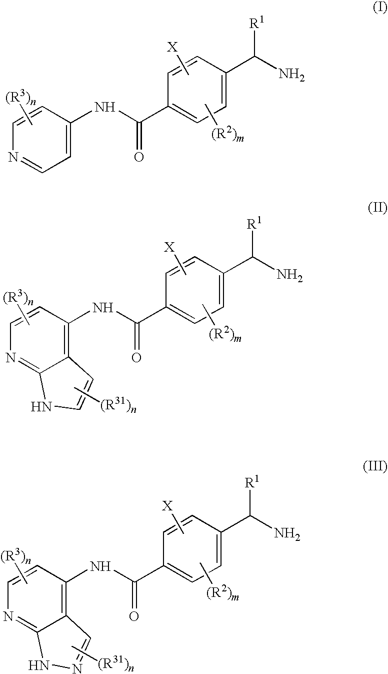

### FIELD OF THE INVENTION

The present invention relates to new kinase inhibitors, more specifically AGC kinases inhibitors, compositions, in particular pharmaceuticals, comprising such inhibitors, and to uses of such inhibitors in the treatment and prophylaxis of disease.

### BACKGROUND OF THE INVENTION

AGC-family protein kinases are named after their family members: protein kinase A (PKA), protein kinase G (PKG), and protein kinase C (PKC).

One AGC-kinase family of interest is Rho-associated coiled-coil forming protein serine/threonine kinase (ROCK), which is believed to be an effector of Ras-related small GTPase Rho. The Rho family consists of at least 10 members of small GTP binding proteins, including RhoA, B, C, D, E, F, G, Rac1, Rac2, Cdc42, and TC10. Two isoforms of ROCK are known: α (ROCKII) and β (ROCKI). ROCKI shows highest expression levels in non-neuronal tissues, such as heart, lung, and skeletal muscles; whereas ROCKII is preferentially expressed in brain (hippocampus, cortex, and cerebellum).

The Rho/Rho-kinase mediated pathway plays an important role in the signal transduction pathway of many agonists such as angiotensin II, 5-HT, NA, thrombin, endothelin-1, urotensin II, platelet-derived growth factor, and ATP/ADP. Activation of ROCK leads to phosphorylation of various proteins: MLCP, MLC, LIMKs, CRMP2, and others. One of the main substrates is the myosin light chain MLC. Activation of MLC, together with the ROCK-induced inactivation of the MLCPhosphatase, leads to stimulation of actin-myosin interactions and subsequent cell contraction and stress fiber formation. ROCK also induces activation of LIMs resulting in an increase of actin filaments. Finally, ROCK activates the ERM protein complex and other proteins involved in cytoskeletal regulation.

ROCK associates with and activates the IKK complex. ROCK inhibitors prevent the degradation of the IKK complex and subsequent NF-κB activation induced by MPS and TNF. As a consequence, ROCK inhibitors decrease NF-κB transcription stimulated by pro-inflammatory mediators. NF-κB is a ubiquitously expressed family of transcription factors controlling the expression of numerous genes involved in immunity and inflammation. Therefore, ROCK inhibitors provide a useful therapy to treat auto-immune and inflammatory diseases.

In conclusion, ROCKs play an important role in various cellular functions: such as smooth muscle contraction, actin cytoskeleton organization, platelet activation, downregulation of myosin phosphatase cell adhesion, -migration, -proliferation, and -survival, thrombin-induced responses of aortic smooth muscle cells, hypertrophy of cardiomyocytes, bronchial smooth muscle contraction, smooth muscle contraction, and cytoskeletal reorganization of non-muscle cells, activation of volume-regulated anion channels, neurite retraction, neutrophil chemotaxis, wound healing, and cell transformation and gene expression.

More specifically, ROCK has been implicated in various diseases and disorders including hypertension, cerebral vasospasm, coronary vasospasm, bronchial asthma, preterm labor, erectile dysfunction, glaucoma, vascular smooth muscle cell proliferation, myocardial hypertrophy, malignoma, ischemia/reperfusion-induced injury, endothelial dysfunction, Crohn's Disease, and colitis, neurite outgrowth, Raynaud's Disease, angina, Alzheimer's disease, benign prostatic hyperplasia, and atherosclerosis.

Accordingly, the development of inhibitors of ROCK would be useful as therapeutic agents for the treatment of disorders implicated in the ROCK pathway. Accordingly, there is a great need to develop inhibitors of ROCK that are useful in treating various diseases or conditions associated with ROCK activation, particularly given the inadequate treatments currently available for the majority of these disorders.

### SUMMARY OF THE INVENTION

We have surprisingly found that the compounds described herein act as inhibitors of AGC-kinases, and in particular of ROCK.

These compounds and pharmaceutically acceptable compositions thereof are useful for treating or lessening the severity of a variety of disorders, including allergic disorders such as asthma, cardiovascular diseases, vascular diseases, eye diseases, renal diseases, erectile dysfunction, inflammatory diseases, proliferative disorders, neurological disorders, and diseases of the central nervous system (CNS), osteoporosis, renal diseases, and AIDS.

Viewed from a first aspect, the invention provides a compound of Formula I, II, or III or a stereoisomer, tautomer, racemate, metabolite, pro- or predrug, salt, hydrate, or solvate thereof,

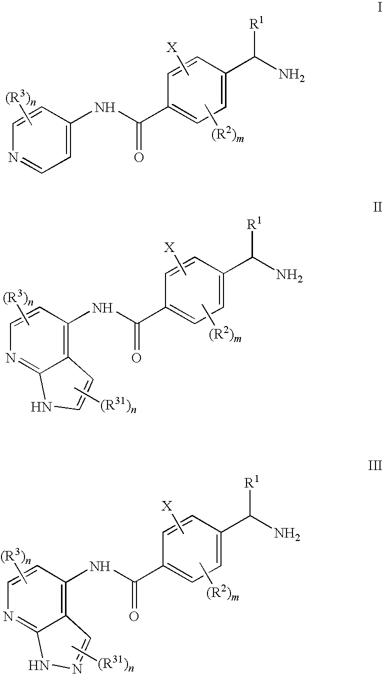

wherein:
X is a group selected from hydroxyl, amino, nitro, alkoxy, alkylamino, hydroxyalkyloxy, aminoalkyloxy, alkynyl, arylalkynyl, heteroarylalkynyl, aryl, heteroaryl, aralkyl, heteroarylalkyl, aryloxy, heteroaryloxy, arylalkoxy, arylaminothiocarbonylamino, heteroarylaminothiocarbonylamino, arylalkylamino, heteroarylalkylamino, arylcarbonylamino, heteroarylcarbonylamino, arylaminocarbonyl, heteroarylaminocarbonyl, arylaminocarbonylamino, or heteroarylaminocarbonylamino, each group being optionally substituted by one or more substituents selected from halogen, hydroxyl, oxo, nitro, amino, aminocarbonyl, azido, cyano, carboxyl, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, arylamino, acyl, arylcarbonyl, aminocarbonyl, alkylsulfoxide, —SO₂R¹⁵, or alkylthio, wherein R¹⁵ is alkyl or cycloalkyl,
R¹ is hydrogen or a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
R² is hydrogen, halogen, nitro, cyano, or hydroxyl, or a group selected from alkyl, alkenyl, alkynyl, amino, acyl, acylamino, alkoxy, arylamino, haloalkoxy, aryl, or heteroaryl, each group being optionally substituted by one or more substituents selected from hydroxyl, halogen, oxo, nitro, amino, cyano, alkyl, alkoxy, haloalkyl, or haloalkoxy, and m is an integer selected from 0, 1, 2, or 3, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is an integer selected from 0, 1, 2, or 3.

Viewed from a further aspect, the invention provides a pharmaceutical and/or veterinary composition comprising a compound of the invention.

Viewed from a still further aspect, the invention provides a compound of the invention for use in human or veterinary medicine.

Viewed from a still further aspect, the invention provides the use of a compound of the invention in the preparation of a medicament for the prevention and/or treatment of at least one disease and/or disorder selected from the group comprising eye diseases; erectile dysfunction; cardiovascular diseases; vascular diseases; proliferative diseases; inflammatory diseases; neurological diseases, and disease of the central nervous system (CNS); bronchial asthma; osteoporosis; renal diseases; and AIDS.

Viewed from a still further aspect, the invention provides the use of a compound of the invention in the preparation of a medicament for the prevention and/or treatment of eyes diseases including macular degeneration, retinopathy, and glaucoma, and/or for preventing, treating and/or alleviating complications and/or symptoms associated therewith.

Viewed from a still further aspect, the invention provides the use of a compound of the invention in the preparation of a medicament for the prevention and/or treatment of inflammatory diseases, such as contact dermatitis, psoriasis, rheumatoid arthritis, inflammatory bowel disease, Crohn's disease, ulcerative colitis, and/or for preventing, treating and/or alleviating complications and/or symptoms and/or inflammatory responses associated therewith.

Viewed from a still further aspect, the invention provides the use of a compound of the invention in the preparation of a medicament for the prevention and/or treatment of cardiovascular and vascular diseases including but not limited to acute stroke, congestive heart failure, cardiovascular ischemia, heart disease, cardiac remodeling, angina, coronary vasospasm, cerebral vasospasm, restenosis, hypertension, (pulmonary) hypertension, arteriosclerosis, thrombosis (including deep thrombosis), pulmonary vasoconstriction, and platelet related diseases, and/or for preventing, treating and/or alleviating complications and/or symptoms associated therewith and/or alleviating complications and/or symptoms associated therewith.

Viewed from a still further aspect, the invention provides the use of a compound of the invention in the preparation of a medicament for the prevention, treatment and/or management of neurological and CNS disorders including but not limited to stroke, multiple sclerosis, spinal cord injury, inflammatory, and demyelinating diseases such as Alzheimer's disease, MS, and neuropathic pain, and/or for preventing, treating and/or alleviating complications and/or symptoms associated therewith.

Viewed from a still further aspect, the invention provides the use of a compound of the invention in the preparation of a medicament for the prevention and/or treatment of proliferative diseases such as including but not limited to cancer of the brain (gliomas), breast, colon, intestine, skin, head and neck, kidney, lung, liver, ovarian, pancreatic, prostate, or thyroid; leukemia; sarcoma; lymphoma; melanoma; and/or for preventing, treating and/or alleviating complications and/or symptoms and/or inflammatory responses associated therewith.

Viewed from a still further aspect, the invention provides the use of a compound of the invention in the preparation of a medicament for the prevention and/or treatment of erectile dysfunction, bronchial asthma, osteoporosis, renal diseases, and AIDS, and/or for preventing, treating and/or alleviating complications and/or symptoms associated therewith.

Viewed from a still further aspect, the invention provides the use of a compound of the invention, or a composition comprising such a compound, for inhibiting the activity of at least one kinase, in vitro or in vivo.

Viewed from a still further aspect, the invention provides the use of a compound of the invention, or a composition comprising such a compound, for inhibiting the activity of at least one ROCK kinase, for example ROCKII and/or ROCKI isoforms.

### BRIEF DESCRIPTION OF THE FIGURES

FIG. 1 represents a graph plotting the percentage of systolic arterial pressure reduction as a function of time for a compound according to the invention falling under formula X, in an embodiment of the present invention. Vehicle (), clonidine (0.3 mg/kg) (◯) positive control, Y-27632 reference compound (▴) (10 mg/kg), example compound at 3 mg/kg (□), 10 mg/kg (▪) and 30 mg/kg (⋄), were tested.

FIG. 2 represents a graph plotting the heart rate as a function of time for a compound according to the invention falling under formula X, in an embodiment of the present invention. Vehicle (), clonidine (0.3 mg/kg) (◯) positive control, Y-27632 reference compound (▴) (10 mg/kg), example compound at 3 mg/kg (□), 10 mg/kg (ζ) and 30 mg/kg (⋄), were tested.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides compound of Formula I, II, or III, wherein:

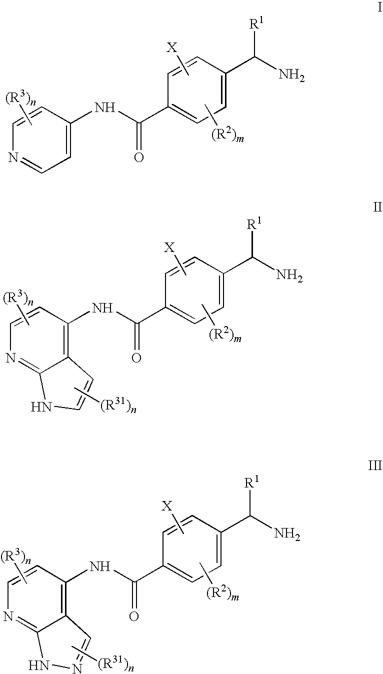

X, R¹, R², R³, R³¹, m, and n have the same meaning as that defined above.

In an embodiment, the present invention relates to compounds of Formula I, II, or III,

X is a group selected from hydroxyl, amino, nitro, C₁₋₆alkoxy, C₁₋₆alkylamino, hydroxyC₁₋₆alkyloxy, aminoC₁₋₆alkyloxy, C₂₋₈alkynyl, C₁₋₁₀arylC₂₋₈alkynyl, heteroarylC₂₋₈alkynyl, C₅₋₁₀aryl, heteroaryl, C₅₋₁₀arylC₁₋₆alkyl, heteroarylC₁₋₆alkyl, C₅₋₁₀aryloxy, heteroaryloxy, arylC₁₋₆alkoxy, C₅₋₁₀ arylaminothiocarbonylamino, heteroarylaminothiocarbonylamino, C₅₋₁₀arylC₁₋₆alkylamino, heteroarylC₁₋₆alkylamino, C₅₋₁₀arylcarbonylamino, heteroarylcarbonylamino, C₅₋₁₀arylaminocarbonyl, heteroarylaminocarbonyl, C₅₋₁₀arylaminocarbonylamino, or heteroarylaminocarbonylamino, each group being optionally substituted by one or more substituents selected from halogen, hydroxyl, oxo, nitro, amino, aminocarbonyl, azido, cyano, carboxyl, C₁₋₆alkyl, C₃₋₈cycloalkyl, C₂₋₈alkenyl, C₂₋₈alkynyl, C₁₋₆cycloalkylC₁₋₆alkyl, C₁₋₆alkylamino, C₁₋₆alkoxy, —SO₂—NH₂, C₅₋₁₀aryl, heteroaryl, arylC₁₋₆alkyl, haloC₁₋₆alkyl, haloC₁₋₄alkoxy, C₁₋₆alkyloxycarbonyl, C₁₋₆alkylaminocarbonyl, heteroarylC₁₋₆alkyl, C₁₋₆alkylsulfonamide, heterocyclyl, C₁₋₆alkylcarbonylaminoC₁₋₆alkyl, C₅₋₁₀aryloxy, C₁₋₆alkylcarbonyl, C₅₋₁₀arylamino, acyl, C₅₋₁₀ arylcarbonyl, aminocarbonyl, C₁₋₆alkylsulfoxide, —SO₂R⁵, or C₁₋₆alkylthio, wherein R¹⁵ is C₁₋₆alkyl or C₁₋₈cycloalkyl,
R¹ is hydrogen or a group selected from C₁₋₆alkyl, C₃₋₈cycloalkyl, C₅₋₁₀aryl, heteroaryl, or heterocyclyl, each group being optionally substituted by one or more substituents selected from hydroxyl, halogen, oxo, nitro, amino, cyano, C₁₋₆alkyl, C₁₋₆alkoxy, haloC₁₋₆alkyl, or haloC₁₋₆alkoxy,
R² is hydrogen, halogen, nitro, cyano, or hydroxyl, or a group selected from C₁₋₆alkyl, C₂₋₈alkenyl, C₂₋₈alkynyl, amino, acyl, acylamino, C₁₋₆alkoxy, arylamino, haloC₁₋₆alkoxy, C₅₋₁₀aryl, or heteroaryl, each group being optionally substituted by one or more substituents selected from hydroxyl, halogen, oxo, nitro, amino, cyano, C₁₋₆alkyl, C₁₋₆alkoxy, haloC₁₋₆alkyl, or haloC₁₋₆alkoxy, and m is an integer selected from 0, 1, 2, or 3, and
R³ and R³¹ are each independently selected from halogen, hydroxyl, oxo, nitro, amino, aminocarbonyl, azido, cyano, C₁₋₆alkyl, C₃₋₈cycloalkyl, C₂₋₈alkenyl, C₂₋₈alkynyl, C₃₋₈cycloalkylC₁₋₆ alkyl, C₁₋₆alkylamino, C₁₋₆alkoxy, —SO₂—NH₂, aryl, heteroaryl, C₅₋₁₀arylC₁₋₆alkyl, haloC₁₋₆alkyl, haloC₁₋₆alkoxy, C₁₋₆alkyloxycarbonyl, C₁₋₆alkylaminocarbonyl, heteroarylC₁₋₆alkyl, C₁₋₆alkylsulfonamide, heterocyclyl, C₁₋₆alkylcarbonylaminoC₁₋₆alkyl, C₅₋₁₀aryloxy, C₁₋₆alkylcarbonyl, acyl, C₅₋₁₀arylcarbonyl, aminocarbonyl, C₁₋₆alkylsulfoxide, —SO₂R¹⁵, or C₁₋₆alkylthio, wherein R¹⁵ is C₁₋₆alkyl or C₃₋₈cycloalkyl, and n is an integer selected from 0, 1, 2, or 3.

Preferably, X is a group selected from hydroxyl, amino, nitro, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, methylamino, ethylamino, n-propylamino, i-propylamino, n-butylamino, i-butylamino, t-butylamino, pentylamino, ethynyl, propynyl, butynyl, pentynyl, hexynyl, methylpropynyl, 4-methyl-1-butynyl, 4-propyl-2-pentynyl, amino-methoxy, 2-amino-ethoxy, 3-amino-propoxy, 4-aminobutoxy, hydroxymethoxy, 2-hydroxy-ethoxy, 3-hydroxy-propoxy, phenyl, biphenylyl, biphenylenyl, 5-tetralinyl, 6-tetralinyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1,2,3,4-tetrahydronaphthyl, 1,4-dihydronaphthyl, 1-pyrenyl, 2-pyrenyl, 3-pyrenyl, 4-pyrenyl, 5-pyrenyl, benzoyl, benzyl, benzoylamino, 3-phenyl-thioureido, 3-phenyl-ureido, 5,8-dihydronaphthalen-1-yl, anilinothiocarbonylamino, benzylamino, benzyloxy, furan-2-ylcarbonylamino, furan-2-ylmethyl-amino, 2-furyl, 3-furyl, 2-thienyl, 3-thienyl, 1-pyrrolyl, 2-pyrrolyl, 3-pyrrolyl, 1-imidazolyl, 2-imidazolyl, 4-imidazolyl, 5-imidazolyl, 1-pyrazolyl, 3-pyrazolyl, 4-pyrazolyl, 5-pyrazolyl, 3-isoxazolyl, 4-isoxazolyl, 5-isoxazolyl, 2-oxazolyl, 4-oxazolyl, 5-oxazolyl, 3-isothiazolyl, 4-isothiazolyl, 5-isothiazolyl, 2-thiazolyl, 4-thiazolyl, 5-thiazolyl, 1,2,3-triazol-1-yl, 1,2,3-triazol-2-yl, 1,2,3-triazol-4-yl, 1,2,3-triazol-5-yl, 1,2,4-triazol-1-yl, 1,2,4-triazol-3-yl, 1,2,4-triazol-4-yl, 1,2,4-triazol-5-yl, 1,2,3-oxadiazol-4-yl, 1,2,3-oxadiazol-5-yl, 1,2,4-oxadiazol-3-, -5-yl, 1,2,5-oxadiazolyl, 1,3,4-oxadiazolyl, 1-tetrazolyl, 5-tetrazolyl, 2-pyridyl, 3-pyridyl, 4-pyridyl, 3-pyridazinyl, 4-pyridazinyl, 2-pyrimidinyl, 4-pyrimidinyl, 5-pyrimidinyl, 6-pyrimidinyl, 2-benzofuryl, 3-benzofuryl, 4-benzofuryl, 5-benzofuryl, 6-benzofuryl, 7-benzofuryl, 2-benzothienyl, 3-benzothienyl, 4-benzothienyl, 5-benzothienyl, 6-benzothienyl, 7-benzothienyl, 1-indolyl, 2-indolyl, 3-indolyl, 4-indolyl, 5-indolyl, 6-indolyl, 7-indolyl, 1,4-oxazin-2-yl, 1,4-oxazin-3-yl, 1,4-dioxin-2-yl, 1,4-dioxin-3-yl, 1,4-thiazin-2-yl, 1,4-thiazin-3-yl, 1,2,3-triazinyl, 1,2,4-triazinyl, 1,3,5-triazin-2-yl, 1,3,5-triazin-4-yl, 1,3,5-triazin-6-yl, 1-benzimidazolyl, 2-benzimidazolyl, 4-benzimidazolyl, 5-benzimidazolyl, 1-benzopyrazolyl, 3-benzopyrazolyl, 4-benzopyrazolyl, 5-benzopyrazolyl, 6-benzopyrazolyl, 7-benzopyrazolyl, 3-benzisoxazolyl, 4-benzisoxazolyl, 5-benzisoxazolyl, 6-benzisoxazolyl, 7-benzisoxazolyl, 2-benzoxazolyl, 4-benzoxazolyl, 5-benzoxazolyl, 6-benzoxazolyl, 7-benzoxazolyl, 3-benzisothiazolyl, 4-benzisothiazolyl, 5-benzisothiazolyl, 6-benzisothiazolyl, 7-benzisothiazolyl, 2-benzothiazolyl, 4-benzothiazolyl, 5-benzothiazolyl, 6-benzothiazolyl, 7-benzothiazolyl, 1-thianthrenyl, 2-thianthrenyl, 3-isobenzofuranyl, 4-isobenzofuranyl, 5-isobenzofuranyl, 2-pyrazinyl, 3-pyrazinyl, 2-isoindolyl, 3-isoindolyl, 4-isoindolyl, 5-isoindolyl, 2-purinyl, 6-purinyl, 7-purinyl, 8-purinyl, 2-quinolyl, 3-quinolyl, 4-quinolyl, 5-quinolyl, 6-quinolyl, 7-quinolyl, 8-quinolyl, 2-quinazolyl, 4-quinazolyl, 5-quinazolyl, 6-quinazolyl, 7-quinazolyl, 8-quinazolyl, 1-isoquinolyl, 3-isoquinolyl, 4-isoquinolyl, 5-isoquinolyl, 6-isoquinolyl, 7-isoquinolyl, 8-isoquinolyl, 3-cinnolinyl, 4-cinnolinyl, 5-cinnolinyl, 6-cinnolinyl, 7-cinnolinyl, 8-cinnolinyl, phenylaminothiocarbonylamino, biphenylylaminothiocarbonylamino, biphenylenylaminothiocarbonylamino, 5-tetralinylaminothiocarbonylamino, 6-tetralinylaminothiocarbonylamino, 1-naphthylaminothiocarbonylamino, 2-naphthylaminothiocarbonylamino, 1-indenylaminothiocarbonylamino, 2-indenylaminothiocarbonylamino, 3-indenylaminothiocarbonylamino, 1-anthrylaminothiocarbonylamino, 2-anthrylaminothiocarbonylamino, 9-anthrylaminothiocarbonylamino, 4-indanylaminothiocarbonylamino, 5-indanylaminothiocarbonylamino, 5-tetrahydronaphthylaminothiocarbonylamino, 6-tetrahydronaphthylaminothiocarbonylamino, 7-tetrahydronaphthylaminothiocarbonylamino, 8-tetrahydronaphthylaminothiocarbonylamino, 1,2,3,4-tetrahydronaphthylaminothiocarbonylamino, 1,4-dihydronaphthylaminothiocarbonylamino, 1-pyrenylaminothiocarbonylamino, 2-pyrenylaminothiocarbonylamino, 3-pyrenylaminothiocarbonylamino, 4-pyrenylaminothiocarbonylamino, 5-pyrenylaminothiocarbonylamino, 2-furylaminothiocarbonylamino, 3-furylaminothiocarbonylamino, 2-thienylaminothiocarbonylamino, 3-thienylaminothiocarbonylamino, 1-pyrrolylaminothiocarbonylamino, 2-pyrrolylaminothiocarbonylamino, 3-pyrrolylaminothiocarbonylamino, 1-pyrazolylaminothiocarbonylamino, 3-pyrazolylaminothiocarbonylamino, 4-pyrazolylaminothiocarbonylamino, 5-pyrazolylaminothiocarbonylamino, 2-thiazolylaminothiocarbonylamino, 4-thiazolylaminothiocarbonylamino, 5-thiazolylaminothiocarbonylamino, 2-pyridylaminothiocarbonylamino, 3-pyridylaminothiocarbonylamino, 4-pyridylaminothiocarbonylamino, 2-pyrimidinylaminothiocarbonylamino, 4-pyrimidinylaminothiocarbonylamino, 5-pyrimidinylaminothiocarbonylamino, 6-pyrimidinylaminothiocarbonylamino, 1-indolylaminothiocarbonylamino, 2-indolylaminothiocarbonylamino, 3-indolylaminothiocarbonylamino, 4-indolylaminothiocarbonylamino, 5-indolylaminothiocarbonylamino, 6-indolylaminothiocarbonylamino, 7-indolylaminothiocarbonylamino, 2-pyrazinylaminothiocarbonylamino, 3-pyrazinylaminothiocarbonylamino, 2-purinylaminothiocarbonylamino, 6-purinylaminothiocarbonylamino, 7-purinylaminothiocarbonylamino, 8-purinylaminothiocarbonylamino, 2-quinolylaminothiocarbonylamino, 3-quinolylaminothiocarbonylamino, 4-quinolylaminothiocarbonylamino, 5-quinolylaminothiocarbonylamino, 6-quinolylaminothiocarbonylamino, 7-quinolylaminothiocarbonylamino, 8-quinolylaminothiocarbonylamino, 2-furylcarbonylamino, 3-furylcarbonylamino, 2-thienylcarbonylamino, 3-thienylcarbonylamino, 1-pyrrolylcarbonylamino, 2-pyrrolylcarbonylamino, 3-pyrrolylcarbonylamino, 1-pyrazolylcarbonylamino, 3-pyrazolylcarbonylamino, 4-pyrazolylcarbonylamino, 5-pyrazolylcarbonylamino, 2-thiazolylcarbonylamino, 4-thiazolylcarbonylamino, 5-thiazolylcarbonylamino, 2-pyridylcarbonylamino, 3-pyridylcarbonylamino, 4-pyridylcarbonylamino, 2-pyrimidinylcarbonylamino, 4-pyrimidinylcarbonylamino, 5-pyrimidinylcarbonylamino, 6-pyrimidinylcarbonylamino, 1-indolylcarbonylamino, 2-indolylcarbonylamino, 3-indolylcarbonylamino, 4-indolylcarbonylamino, 5-indolylcarbonylamino, 6-indolylcarbonylamino, 7-indolylcarbonylamino, 2-pyrazinylcarbonylamino, 3-pyrazinylcarbonylamino, 2-purinylcarbonylamino, 6-purinylcarbonylamino, 7-purinylcarbonylamino, 8-purinylcarbonylamino, 2-quinolylcarbonylamino, 3-quinolylcarbonylamino, 4-quinolylcarbonylamino, 5-quinolylcarbonylamino, 6-quinolylcarbonylamino, 7-quinolylcarbonylamino, 8-quinolylcarbonylamino, phenylcarbonylamino, biphenylylcarbonylamino, biphenylenylcarbonylamino, 1-naphthylcarbonylamino, 2-naphthylcarbonylamino, 1-indenylcarbonylamino, 2-indenylcarbonylamino, 3-indenylcarbonylamino, 4-indanylcarbonylamino, 5-indanylcarbonylamino, 5-tetrahydronaphthylcarbonylamino, 6-tetrahydronaphthylcarbonylamino, 7-tetrahydronaphthylcarbonylamino, 8-tetrahydronaphthylcarbonylamino, 1,2,3,4-tetrahydronaphthylcarbonylamino, 1,4-dihydronaphthylcarbonylamino, 1-pyrenylcarbonylamino, 2-pyrenylcarbonylamino, 3-pyrenylcarbonylamino, 4-pyrenylcarbonylamino, or 5-pyrenylcarbonylamino,

each group being optionally substituted by one, two, or three substituents selected from methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloro, fluoro, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, hydroxyl, oxo, nitro, cyano, amino, aminocarbonyl, carboxyl, phenyl, 1-naphthyl, 2-naphthyl, 2-thienyl, 3-thienyl, 2-furyl, 3-furyl, hydroxymethyl, 2-hydroxyethyl, phenyloxy, biphenylyloxy, biphenylenyloxy, 1-naphthyloxy, 2-naphthyloxy, methylamino, ethylamino, n-propylamino, i-propylamino, n-butylamino, i-butylamino, t-butylamino, pentylamino, or hexylamino, and R¹, R², R³, R³¹, m, and n have the same meaning as that defined above.

Preferably X is a group selected from hydroxyl, amino, nitro, ethynyl, propynyl, butynyl, pentynyl, hexynyl, methylpropynyl, 4-methyl-1-butynyl, 4-propyl-2-pentynyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, amino-methoxy, 2-amino-ethoxy, 3-amino-propoxy, 4-aminobutoxy, 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-thiazolyl, 4-thiazolyl, 5-thiazolyl, 2-pyridyl, 3-pyridyl, 4-pyridyl, 2-pyrimidinyl, 4-pyrimidinyl, 5-pyrimidinyl, 6-pyrimidinyl, 1-indolyl, 2-indolyl, 3-indolyl, 4-indolyl, 5-indolyl, 6-indolyl, 7-indolyl, 2-pyrazinyl, 3-pyrazinyl, 2-purinyl, 6-purinyl, 7-purinyl, 8-purinyl, 2-quinolyl, 3-quinolyl, 4-quinolyl, 5-quinolyl, 6-quinolyl, 7-quinolyl, 8-quinolyl, hydroxymethoxy, 2-hydroxy-ethoxy, 3-hydroxy-propoxy, phenyl, biphenylyl, biphenylenyl, 1-naphthyl, 2-naphthyl, 1-indenyl, 2-indenyl, 3-indenyl, 4-indanyl, 5-indanyl, 5-tetrahydronaphthyl, 6-tetrahydronaphthyl, 7-tetrahydronaphthyl, 8-tetrahydronaphthyl, 1,2,3,4-tetrahydronaphthyl, 1,4-dihydronaphthyl, 1-pyrenyl, 2-pyrenyl, 3-pyrenyl, 4-pyrenyl, 5-pyrenyl, 3,4-dichloro-phenyl, 3-chloro-benzoylamino, 2′-chloro-phenyl, 2′-methoxy-phenyl, 3-chloro-phenyl, 3-cyano-phenyl, 3-methoxy-benzoylamino, 3-methoxy-phenyl, 3-phenyl-thioureido, 3-phenyl-ureido, 4-chloro-benzoylamino, 4-chloro-phenyl, 4-cyano-phenyl, 4-fluoro-phenyl, 4-hydroxymethyl-phenyl, 4-hydroxy-phenyl, 4-methoxy-benzoylamino, 4-methoxy-phenyl, 5,8-dihydronaphthalen-1-yl, anilinothiocarbonylamino, benzoylamino, benzylamino, benzyloxy, furan-2-ylcarbonylamino, furan-2-ylmethyl-amino, phenylaminothiocarbonylamino, biphenylylaminothiocarbonylamino, biphenylenylaminothiocarbonylamino, 1-naphthylaminothiocarbonylamino, 2-naphthylaminothiocarbonylamino, 1-indenylaminothiocarbonylamino, 2-indenylaminothiocarbonylamino, 3-indenylaminothiocarbonylamino, 4-indanylaminothiocarbonylamino, 5-indanylaminothiocarbonylamino, 5-tetrahydronaphthylaminothiocarbonylamino, 6-tetrahydronaphthylaminothiocarbonylamino, 7-tetrahydronaphthylaminothiocarbonylamino, 8-tetrahydronaphthylaminothiocarbonylamino, 1,2,3,4-tetrahydronaphthylaminothiocarbonylamino, 1,4-dihydronaphthylaminothiocarbonylamino, 2-furylaminothiocarbonylamino, 3-furylaminothiocarbonylamino, 2-thienylaminothiocarbonylamino, 3-thienylaminothiocarbonylamino, 1-pyrrolylaminothiocarbonylamino, 2-pyrrolylaminothiocarbonylamino, 3-pyrrolylaminothiocarbonylamino, 1-pyrazolylaminothiocarbonylamino, 3-pyrazolylaminothiocarbonylamino, 4-pyrazolylaminothiocarbonylamino, 5-pyrazolylaminothiocarbonylamino, 2-pyridylaminothiocarbonylamino, 3-pyridylaminothiocarbonylamino, 4-pyridylaminothiocarbonylamino, 2-pyrimidinylaminothiocarbonylamino, 4-pyrimidinylaminothiocarbonylamino, 5-pyrimidinylaminothiocarbonylamino, 6-pyrimidinylaminothiocarbonylamino, 1-indolylaminothiocarbonylamino, 2-indolylaminothiocarbonylamino, 3-indolylaminothiocarbonylamino, 4-indolylaminothiocarbonylamino, 5-indolylaminothiocarbonylamino, 6-indolylaminothiocarbonylamino, 7-indolylaminothiocarbonylamino, 2-pyrazinylaminothiocarbonylamino, 3-pyrazinylaminothiocarbonylamino, 2-purinylaminothiocarbonylamino, 6-purinylaminothiocarbonylamino, 7-purinylaminothiocarbonylamino, 8-purinylaminothiocarbonylamino, 2-furylcarbonylamino, 3-furylcarbonylamino, 2-thienylcarbonylamino, 3-thienylcarbonylamino, 1-pyrrolylcarbonylamino, 2-pyrrolylcarbonylamino, 3-pyrrolylcarbonylamino, 1-pyrazolylcarbonylamino, 3-pyrazolylcarbonylamino, 4-pyrazolylcarbonylamino, 5-pyrazolylcarbonylamino, 2-pyridylcarbonylamino, 3-pyridylcarbonylamino, 4-pyridylcarbonylamino, 2-pyrimidinylcarbonylamino, 4-pyrimidinylcarbonylamino, 5-pyrimidinylcarbonylamino, 6-pyrimidinylcarbonylamino, 1-indolylcarbonylamino, 2-indolylcarbonylamino, 3-indolylcarbonylamino, 4-indolylcarbonylamino, 5-indolylcarbonylamino, 6-indolylcarbonylamino, 7-indolylcarbonylamino, 2-pyrazinylcarbonylamino, 3-pyrazinylcarbonylamino, 2-purinylcarbonylamino, 6-purinylcarbonylamino, 7-purinylcarbonylamino, 8-purinylcarbonylamino, phenylcarbonylamino, biphenylylcarbonylamino, biphenylenylcarbonylamino, 1-naphthylcarbonylamino, 2-naphthylcarbonylamino, 1-indenylcarbonylamino, 2-indenylcarbonylamino, 3-indenylcarbonylamino, 4-indanylcarbonylamino, 5-indanylcarbonylamino, 5-tetrahydronaphthylcarbonylamino, 6-tetrahydronaphthylcarbonylamino, 7-tetrahydronaphthylcarbonylamino, 8-tetrahydronaphthylcarbonylamino, 1,2,3,4-tetrahydronaphthylcarbonylamino, 1,4-dihydronaphthylcarbonylamino, each group being optionally substituted by one or two substituents selected from methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloro, fluoro, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, oxohydroxyl, nitro, cyano, amino, aminocarbonyl, phenyl, 1-naphthyl, 2-naphthyl, 2-thienyl, 3-thienyl, 2-furyl, 3-furyl, hydroxymethyl, 2-hydroxyethyl, phenyloxy, biphenylyloxy, biphenylenyloxy, 1-naphthyloxy, 2-naphthyloxy, and R¹, R², R³, R³¹, m, and n have the same meaning as that defined above.

Unless a context dictates otherwise, asterisks are used herein to indicate the point at which a mono- or bivalent radical depicted is connected to the structure to which it relates and of which the radical forms part.

When describing the compounds of the invention, the terms used are to be construed in accordance with the following definitions, unless a context dictates otherwise:

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 20 carbon atoms, more preferably from 1 to 10 carbon atoms, still more preferably 1 to 8 carbon atoms, in particular 1 to 6 carbon atoms, preferably 1 to 4 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers. C₁-C₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3-, or 4-methylcyclopentyl, cyclopentylmethylene, and cyclohexyl.

The term “optionally substituted alkyl” refers to an alkyl group optionally substituted with one or more substituents (for example 1 to 4 substituents, or example 1, 2, 3, or 4 substituents or 1 to 2 substituents) at any available point of attachment. Non-limiting examples of such substituents include halogen, hydroxyl, carbonyl, nitro, amino, oximes, imines, azido, hydrazino, cyano, alkyl, aryl, heteroaryl, cycloalkyl, acyl, alkylamino, alkoxy, thio, alkylthio, carboxylic acid, acylamino, alkyl esters, carbamates, thioamides, urea, sulphonamides, and the like.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” refers to a —R^{a}—OH group wherein R^{a} is alkylene as defined herein. For example, “hydroxyalkyl” includes 2-hydroxyethyl, 1-(hydroxymethyl)-2-methylpropyl, 3,4-dihydroxybutyl, and so forth. “Alkoxyalkyl” refers to an alkyl group substituted with one to two of OR′, wherein R′ is alkoxy as defined below. For example, “aralkyl” or “arylalkyl” refers to a substituted alkyl group as defined above wherein at least one of the alkyl substituents is an aryl as defined below, such as benzyl. For example, “heteroarylalkyl” refers to a substituted alkyl group as defined above wherein at least one of the alkyl substituents is a heteroaryl as defined below, such as pyridinyl.

The term “cycloalkyl group” as used herein is a cyclic alkyl group, that is to say, a monovalent, hydrocarbyl group having 1, 2, or 3 cyclic structure. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. The further rings of multi-ring cycloalkyls may be either fused, bridged and/or joined through one or more spiro atoms. Cycloalkyl groups may also be considered to be a subset of homocyclic rings discussed hereinafter. Examples of cycloalkyl groups, are cyclopropyl, cyclobutyl, cyclopentyl, cyclohexyl, cycloheptyl, cyclooctyl, cyclononyl, and cyclodecyl with cyclopropyl being particularly preferred. An “optionally substituted cycloalkyl” refers to a cycloalkyl having optionally one or more substituents (for example 1 to 3 substituents, or 1 to 2 substituents), selected from those defined above for substituted alkyl. When the suffix “ene” is used in conjunction with a cyclic group, this is intended to mean the cyclic group as defined herein having two single bonds as points of attachment to other groups.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene, and hexamethylene. Similarly, where alkenyl groups as defined above and alkynyl groups as defined above, respectively, are divalent radicals having single bonds for attachment to two other groups, they are termed “alkenylene” and “alkynylene” respectively.

Generally, alkylene groups of this invention preferably comprise the same number of carbon atoms as their alkyl counterparts. “Cycloalkylene” herein refers to a saturated homocyclic hydrocarbyl biradical of Formula CₙH₂ₙ₋. Cycloalkylene groups of this invention preferably comprise the same number of carbon atoms as their cycloalkyl radical counterparts. Where an alkylene or cycloalkylene biradical is present, connectivity to the molecular structure of which it forms part may be through a common carbon atom or different carbon atom, preferably a common carbon atom. To illustrate this applying the asterisk nomenclature of this invention, a C₃ alkylene group may be for example *—CH₂CH₂CH₂—*, *—CH(—CH₂CH₃)—*, or *—CH₂CH(—CH₃)—*. Likewise a C₃ cycloalkylene group may be

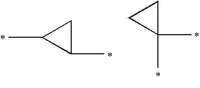

Where a cycloalkylene group is present, this is preferably a C₃-C₆ cycloalkylene group, more preferably a C₃ cycloalkylene (i.e. cyclopropylene group) wherein its connectivity to the structure of which it forms part is through a common carbon atom. Cycloalkylene and alkylene biradicals in compounds of the invention may be, but preferably are not, substituted.

The term “alkenyl” as used herein refers to an unsaturated hydrocarbyl group, which may be linear, branched, or cyclic, comprising one or more carbon-carbon double bonds. Alkenyl groups thus comprise two or more carbon atoms, preferably between 2 and 20 carbon atoms, more preferably between 2 and 10 carbon atoms, still more preferably between 2 and 8 carbon atoms, for example, between 2 and 6 carbon atoms. Similarly to cycloalkyl groups, cycloalkenyl groups may be considered to be a subset of homocyclic rings discussed hereinafter. Examples of alkenyl groups are ethenyl, 2-propenyl, 2-butenyl, 3-butenyl, 2-pentenyl and its isomers, 2-hexenyl and its isomers, 2-heptenyl and its isomers, 2-octenyl and its isomers, 2,4-pentadienyl and the like. An optionally substituted alkenyl refers to an alkenyl having optionally one or more substituents (for example 1, 2, or 3 substituents, or 1 to 2 substituents), selected from those defined above for substituted alkyl. Similarly to cycloalkyl groups, cycloalkenyl groups may be considered to be a subset of homocyclic rings discussed hereinafter.

The term “alkynyl” as used herein, similarly to alkenyl, refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Examples alkynyl groups are ethynyl, 2-propynyl, 2-butynyl, 3-butynyl, 2-pentynyl and its isomers, 2-hexynyl and its isomers, 2-heptynyl and its isomers, 2-octynyl and its isomers and the like, preferably ethynyl, propynyl, butynyl, pentynyl, hexynyl, methylpropynyl, 4-methyl-1-butynyl, or 4-propyl-2-pentynyl-. An optionally substituted alkynyl refers to an alkynyl having optionally one or more substituents (for example 1 to 4 substituents, or 1 to 2 substituents), selected from those defined above for substituted alkyl. Similarly to cycloalkyl groups, cycloalkynyl groups may be considered to be a subset of homocyclic rings discussed hereinafter.

The term “homocyclic ring” as used herein is a ring wherein the ring atoms comprise only carbon atoms. Examples of homocyclic rings thus include cycloalkyl, cycloalkenyl, and cycloalkynyl, with cycloalkyl and cycloalkenyl being preferred. Where a ring carbon atom is replaced with a heteroatom, preferably nitrogen, oxygen of sulfur, the heteroatom-containing ring resultant from such a replacement is referred to herein as a heterocyclic ring. More than one carbon atom in a ring may be replaced so forming heterocyclic ring having a plurality of heteroatoms.

The terms “heterocyclyl” or “heterocyclo” as used herein by itself or as part of another group refer to non-aromatic, fully saturated, or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An optionally substituted heterocyclic refers to a heterocyclic having optionally one or more substituents (for example 1 to 4 substituents, or for example 1, 2, 3, or 4), selected from those defined above for substituted aryl.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, triazinyl, cinnolinyl, phthalazinyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl.

The term “aryl” as used herein refers to a polyunsaturated, aromatic hydrocarbyl group having a single ring (i.e. phenyl) or multiple aromatic rings fused together (e.g. naphthalene or anthracene) or linked covalently, typically containing 5 to 8 atoms; wherein at least one ring is aromatic. The aromatic ring may optionally include one to three additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7-, or 8-azulenyl, 1- or 2-naphthyl, 1-, 2-, or 3-indenyl, 1-, 2-, or 9-anthryl, 1-2-, 3-, 4-, or 5-acenaphtylenyl, 3-, 4-, or 5-acenaphtenyl, 1-, 2-, 3-, 4-, or 10-phenanthryl, 1- or 2-pentalenyl, 1,2-, 3-, or 4-fluorenyl, 4- or 5-indanyl, 5-, 6-, 7-, or 8-tetrahydronaphthyl, 1,2,3,4-tetrahydronaphthyl, 1,4-dihydronaphthyl, dibenzo[a,d]cylcoheptenyl, and 1-, 2-, 3-, 4-, or 5-pyrenyl.

The aryl ring can optionally be substituted by one or more aromatic substituents. An “optionally substituted aryl” refers to an aryl having optionally one or more substituents (for example 1 to 5 substituents, or 1 to 2 substituents) at any available point of attachment. Non-limiting examples of such substituents are selected from halogen, hydroxyl, oxo, nitro, amino, hydrazine, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, alkylthio, carboxyl, and the like, wherein R¹⁵ is alkyl or cycloalkyl.

The term “arylene” as used herein is intended to include divalent carbocyclic aromatic ring systems such as phenylene, biphenylylene, naphthylene, anthracenylene, phenanthrenylene, fluorenylene, indenylene, pentalenylene, azulenylene, and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene, and the like.

Where a carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl, or heterocyclyl ring. An “optionally substituted heteroaryl” refers to a heteroaryl having optionally one or more substituents (for example 1 to 4 substituents, or 1 to 2 substituents), selected from those defined above for substituted aryl.

Non-limiting examples of heteroaryl can be 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, 1,2,3-triazol-1-, -2-, 4-, or -5-yl, 1,2,4-triazol-1-, -3-, 4-, or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3- or 4-pyridyl, 3- or 4-pyridazinyl, 2-, 4-, 5- or 6-pyrimidinyl, 2-, 3-, 4-, 5-6-2H-thiopyranyl, 2-, 3-, or 4-4H-thiopyranyl, 2-, 3-, 4-, 5-, 6-, or 7-benzofuryl, 1-, 3-, 4-, or 5-isobenzofuryl, 2-, 3-, 4-, 5-, 6-, or 7-benzothienyl, 1-, 3-, 4-, or 5-isobenzothienyl, 1-, 2-, 3-, 4-, 5-, 6-, or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, 4-, or -6-yl, thieno[2,3-b]furan-2-, -3-, -4-, or -5-yl, 1-, 2-, 4-, or 5-benzimidazolyl, 1-, 3-, 4-, 5-, 6-, or 7-benzopyrazolyl, 3-, 4-, 5-, 6-, or 7-benzisoxazolyl, 2-, 4-, 5-, 6-, or 7-benzoxazolyl, 3-, 4-, 5-, 6-, or 7-benzisothiazolyl, 2-, 4-, 5-, 6-, or 7-benzothiazolyl, 1-, 2-thianthrenyl, 3-, 4- or 5-isobenzofuranyl, 1-, 2-, 3-, 4-, or 9-xanthenyl, 1-, 2-, 3-, or 4-phenoxathiinyl, 2-, 3-pyrazinyl, 1-, 2-, 3-, 4-, 5-, 6-, 7-, or 8-indolizinyl, 2-, 3-, 4-, or 5-isoindolyl, 1-, 2-, 3-, 4-, 5-, 6-, or 7-indazolyl, 2-, 6-, 7-, or 8-purinyl, 4-, 5-, or 6-phthalazinyl, 2-, 3-, or 4-naphthyridinyl, 2-, 5-, or 6-quinoxalinyl, 2-, 4-, 5-, 6-, 7-, or 8-quinazolinyl, 1-, 2-, 3-, or 4-quinolizinyl, 2-, 3-, 4-, 5-, 6-, 7-, or 8-quinolinyl(quinolyl), 2-, 4-, 5-, 6-, 7-, or 8-quinazolyl, 1-, 3-, 4-, 5-, 6-, 7-, or 8-isoquinolinyl(isoquinolyl), 3-, 4-, 5-, 6-, 7-, or 8-cinnolinyl, 2-, 4-, 6-, or 7-pteridinyl, 1-, 2-, 3-, 4-, or 9-carbazolyl, 1-, 2-, 3-, 4-, 5-, 6-, 7-, 8-, or 9-carbolinyl, 1-, 2-, 3-, 4-, 5-, 6-, 7-, 8-, 9-, or 10-phenanthridinyl, 1-, 2-, 3-, or 4-acridinyl, 1-, 2-, 3-, 4-, 5-, 6-, 7-, 8-, or 9-perimidinyl, 2-, 3-, 4-, 5-, 6-, 7-, 8-, 9-, or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, or additionally substituted derivatives thereof.

The term “oxo” as used herein refers to the group ═O.

The term “alkoxy” as used herein refers to a radical having the Formula —OR wherein R is alkyl. Preferably, alkoxy is C₁-C₁₀ alkoxy, more preferably C₁-C₈ alkoxy, yet more preferably C₁-C₆ alkoxy. Where the oxygen atom in an alkoxy group is substituted with sulfur, the resultant radical is referred to as thioalkoxy. Haloalkoxy is an alkoxy group wherein one or more hydrogen atoms in the alkyl group are substituted with halo.

The term “aryloxy” as used herein denotes a group —O-aryl, wherein aryl is as defined above.

The term “aroyl” as used herein denotes a group —C(O)-aryl, wherein aryl is as defined above.

The term “cycloalkylalkyl” by itself or as part of another substituent refers to a group having one of the aforementioned cycloalkyl groups attached to one of the aforementioned alkyl chains. Examples of such cycloalkylalkyl radicals include cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, 3-cyclopentylbutyl, cyclohexylbutyl, and the like.

The term “heterocyclyl-alkyl” by itself or as part of another substituents refers to a group having one of the aforementioned heterocyclyl group attached to one of the aforementioned alkyl group, i.e., to a group —R^{b}—R^{c} wherein R^{b} is alkylene or alkylene substituted by alkyl group and R^{c} is a heterocyclyl group.

The term “acyl” by itself or as part of another substituent refers to an alkanoyl group having 2 to 6 carbon atoms or a phenylalkanoyl group whose alkanoyl moiety has 1 to 4 carbon atoms, i.e. a carbonyl group linked to a radical such as, but not limited to, alkyl, aryl, more particularly, the group —COR¹⁰, wherein R¹⁰ can be selected from alkyl, aryl, substituted alkyl, or substituted aryl, as defined herein. The term acyl therefore encompasses the group alkylcarbonyl (—COR¹⁰), wherein R¹⁰ is alkyl. Preferably, acyl is C₂-C₁₁ acyl or C₂-C₇ acyl. Where the oxygen atom is an acyl group is substituted with sulfur, the resultant radical is referred to as thioacyl. Said acyl can be exemplified by acetyl, propionyl, butyryl, valeryl, pivaloyl, benzoyl, phenylacetyl, phenylpropionyl, and phenylbutylyl.

The term “amino” refers to the group —NH₂.

The term “alkylamino” by itself or as part of another substituent refers to a group consisting of an amino groups attached to one or two independently selected and optionally substituted alkyl groups, cycloalkyl groups, aralkyl, or cycloalkylalkyl groups i.e., alkyl amino refers to —N(R⁸)(R⁹) wherein R⁸ and R⁹ are each independently selected from hydrogen, cycloalkyl, aralkyl, cycloalkylalkyl, or alkyl. Non-limiting examples of alkylamino groups include methylamino (NHCH₃), ethylamino (NHCH₂CH₃), n-propylamino, isopropylamino, n-butylamino, isobutylamino, sec-butylamino, tert-butylamino, n-hexylamino, and the like.

The term “aminoalkyl” refers to the group —R^{b}—NR^{d}R^{e} wherein R^{b} is alkylene or substituted alkylene, R^{d} is hydrogen, alkyl, or substituted alkyl as defined herein, and R^{e} is hydrogen or alkyl as defined herein.

The term “aminocarbonyl” refers to the group —(C═O)—NH₂.

The term “alkylaminocarbonyl” refers to a group —(C═O)—NR^{d}R^{e} wherein R^{d} is hydrogen, alkyl, or substituted alkyl as defined herein, and R^{e} is alkyl or substituted alkyl as defined herein.

The term “alkylaminocarbonylamino” refers to a group —NH(C═O)—NR^{d}R^{e} or —NR′(C═O)—NR^{d}R^{e} wherein R^{d} is hydrogen, alkyl, or substituted alkyl, as defined herein, R^{e} is alkyl or substituted alkyl, and R′ is alkyl or substituted alkyl, as defined herein.

The term “carboxy” or “carboxyl” refers to the group —CO₂H. Thus, a carboxyalkyl is an alkyl group as defined above having at least one substituent that is —CO₂H.

The term “alkoxycarbonyl” refers to a carboxy group linked to an alkyl radical i.e. to form —C(═O)OR¹⁰, wherein R¹⁰ is as defined above for acyl.

The term “alkylcarbonyloxy” refers to a —O—C(═O)R¹¹ wherein R¹¹ is as defined above for acyl.

The term “alkylcarbonylamino” refers to an group of Formula —NH(C═O)R′ or —NR′(C═O)R, wherein R and R′ are each independently alkyl or substituted alkyl.

The term “alkylcarbonylaminoalkyl” refers to a group of Formula —R^{b}—NR^{d}—C(═O)—R^{e} wherein R^{b} is alkylene or substituted alkylene, R^{d} is hydrogen or alkyl as defined herein, and R^{e} is alkyl as defined herein.

The term “thiocarbonyl” refers to the group —C(═S)—.

The term “arylaminothiocarbonylamino” refers to a group of Formula —NR^{f}—C(═S)—NR^{g}R^{h} wherein R^{f} is selected from hydrogen or alkyl, R^{g} is selected from hydrogen, aryl, or alkyl, and R^{h} is aryl as defined herein.

The term “alkoxy” by itself or as part of another substituent refers to a group consisting of an oxygen atom attached to one optionally substituted straight or branched alkyl group, cycloalkyl group, aralkyl, or cycloalkylalkyl group. Non-limiting examples of suitable alkoxy group include methoxy, ethoxy, n-propoxy, isopropoxy, n-butoxy, iso-butoxy, sec-butoxy, tert-butoxy, hexanoxy, and the like.

The term “alkylthio” by itself or as part of another substituent refers to a group consisting of a sulfur atom attached to one optionally substituted alkyl group, cycloalkyl group, aralkyl, or cycloalkylalkyl group. Non-limiting examples of alkylthio groups include methylthio (SCH₃), ethylthio (SCH₂CH₃), n-propylthio, isopropylthio, n-butylthio, isobutylthio, sec-butylthio, tert-butylthio, n-hexylthio, and the like.

The term “acylamino” by itself or as part of another substituent refers to a group consisting of an amino group attached to one or two independently selected acyl groups as described before. In case the two acyl groups of a dicarboxylic acid are attached to the amino group these represent imides such as phtalimides, maleimides, and the like, and are encompassed in the meaning of the term acylamino.

The term “halo” or “halogen” as a group or part of a group is generic for fluoro, chloro, bromo, or iodo.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl, and the like.

The term “haloalkoxy” alone or in combination refers to a group of Formula —O-alkyl wherein the alkyl group is substituted by 1, 2, or 3 halogen atoms. For example, “haloalkoxy” includes —OCF₃, —OCHF₂, —OCH₂F, —O—CF₂—CF₃, —O—CH₂—CF₃, —O—CH₂—CHF₂, and —O—CH₂—CH₂F.

The term “sulfonamide” alone or in combination refers to a group of Formula —SO₂—NRR wherein each R independently is hydrogen or alkyl as defined herein.

The term “alkylsulfonylamino” alone or in combination refers to a group of Formula —NR^{d}—SO₂—R wherein R^{d} is hydrogen or alkyl as defined herein, and R is alkyl as defined herein.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Where groups may be optionally substituted, such groups may be substituted with once or more, and preferably once or twice. Substituents may be selected from, for example, the group comprising halo, hydroxyl, oxo, nitro, amido, carboxy, amino, cyano haloalkoxy, and haloalkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl and optionally substituted cycloalkyl.

Whenever used in the present invention the term “compounds of the invention” or a similar term is meant to include the compounds of general Formula I, II, and III and any subgroup thereof. This term also refers to the compounds as depicted in Tables 1, 2, and 3 and their derivatives, N-oxides, salts, solvates, hydrates, stereoisomeric forms, racemic mixtures, tautomeric forms, optical isomers, analogues, pro-drugs, esters, and metabolites, as well as their quaternized nitrogen analogues. The N-oxide forms of said compounds are meant to comprise compounds wherein one or several nitrogen atoms are oxidized to the so-called N-oxide.

As used in the specification and the appended claims, the singular forms “a”, “an”, and “the” include plural referents unless the context clearly dictates otherwise. By way of example, “a compound” means one compound or more than one compound.

The terms described above and others used in the specification are well understood to those in the art.

Preferred features of the compounds of this invention are now set forth. According to a preferred embodiment, the present invention provides compounds of Formula I, II, or III, wherein:

X is a group selected from hydroxyl, amino, nitro, alkoxy, alkylamino, hydroxyalkyloxy, aminoalkyloxy, alkynyl, arylalkynyl, heteroarylalkynyl, aryl, heteroaryl, aralkyl, heteroarylalkyl, aryloxy, heteroaryloxy, arylalkoxy, arylaminothiocarbonylamino, heteroarylaminothiocarbonylamino, arylalkylamino, heteroarylalkylamino, arylcarbonylamino, heteroarylcarbonylamino, arylaminocarbonyl, heteroarylaminocarbonyl, arylaminocarbonylamino, or heteroarylaminocarbonylamino, each group being optionally substituted by one or more substituents selected from halogen, hydroxyl, nitro, cyano, amino, alkyl, alkoxy, aryl, arylamino, heteroaryl, haloalkyl, haloalkoxy, heterocyclyl, or aryloxy,
R¹ is a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
R² is hydrogen, halogen, nitro, cyano, or hydroxyl, or a group selected from alkyl, alkenyl, alkynyl, amino, acyl, acylamino, alkoxy, arylamino, haloalkoxy, aryl, or heteroaryl, each group being optionally substituted by one or more substituents selected from hydroxyl, halogen, oxo, nitro, amino, cyano, alkyl, alkoxy, haloalkyl, or haloalkoxy, m is 0, 1, or 2, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, 2, or 3.

According to another preferred embodiment, the present invention provides compounds of Formula I, II, or III, wherein X is selected from nitro or a group selected from hydroxyl, alkoxy, amino, alkylamino, hydroxyalkyloxy, aminoalkyloxy, alkynyl, arylalkynyl, heteroarylalkynyl, aryl, heteroaryl, aralkyl, heteroarylalkyl, aryloxy, arylalkoxy, heteroaryloxy, arylaminothiocarbonylamino, heteroarylaminothiocarbonylamino, arylalkylamino, heteroarylalkylamino, arylcarbonylamino, heteroarylcarbonylamino, arylaminocarbonyl, heteroarylaminocarbonyl, arylaminocarbonylamino, or heteroarylaminocarbonylamino, each group being optionally substituted by one, two or three substituents selected from halogen, hydroxyl, oxo, nitro, amino, aminocarbonyl, azido, cyano, carboxy, alkyl, cycloalkyl, alkenyl, alkynyl, cycloalkylalkyl, alkylamino, alkoxy, —SO₂—NH₂, aryl, heteroaryl, aralkyl, arylamino, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and R¹, R², R³, R³¹, n, m have the same meaning as that defined hereinabove. Preferably, R¹ is nitro or a group selected from hydroxyl, amino, aryl, heteroaryl, hydroxyalkoxy, aminoalkoxy, arylalkoxy, arylaminothiocarbonylamino, arylaminocarbonylamino, arylalkylamino, heteroarylalkylamino, arylcarbonylamino, heteroarylcarbonylamino, alkynyl, each group being optionally substituted by one, two or three substituents selected from halogen, alkoxy, hydroxyl, amino, aryl, arylamino, aralkyl, heteroaryl, heteroarylalkyl, or arylcarbonyl, and R¹, R², R³, R³¹, n, m have the same meaning as that defined hereinabove.

According to another preferred embodiment, the present invention provides compounds having one of the structural Formula IV, V, VI, VII, VIII, IX, X, XI, or XII, wherein:

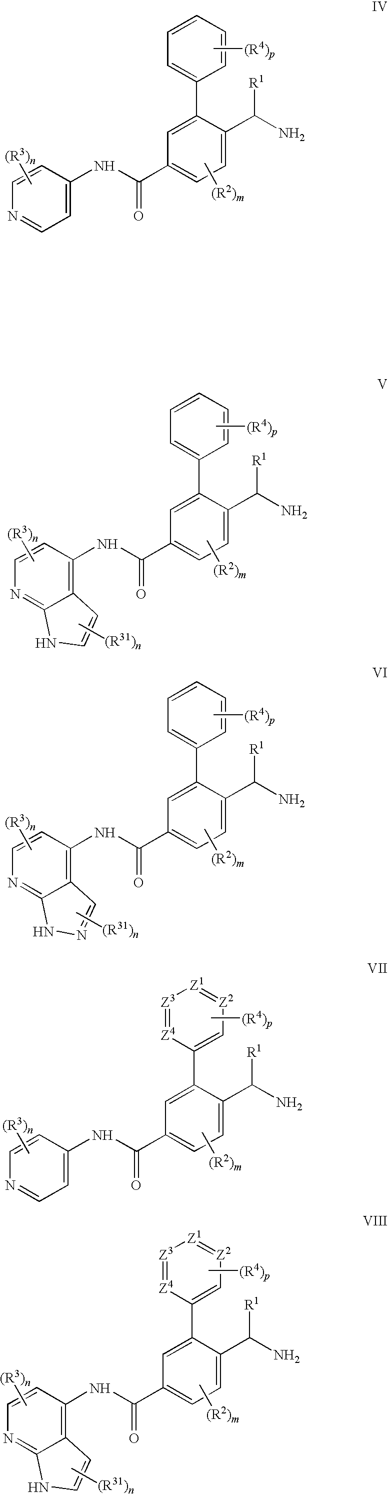

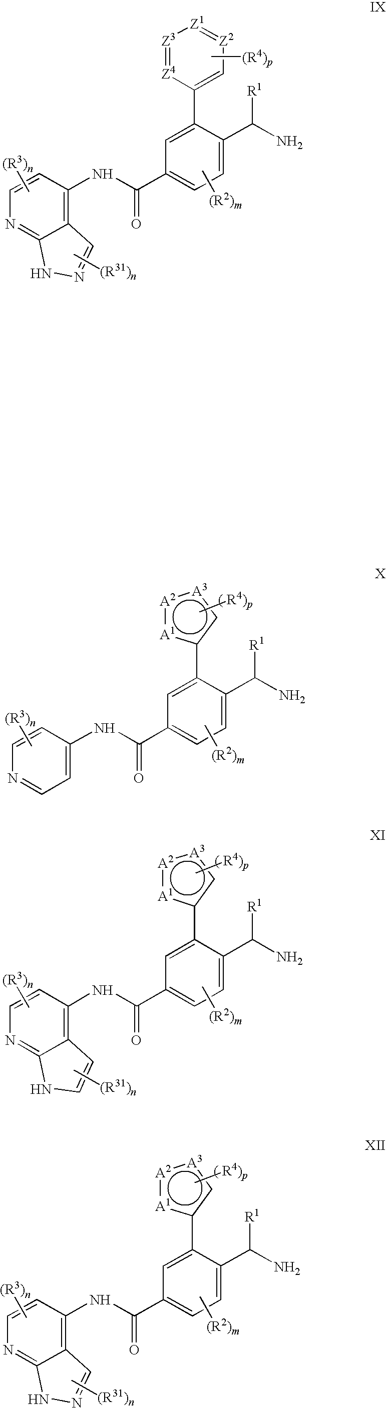

Z¹, Z², Z³, Z⁴ are each independently selected from CH or N, wherein at least one of Z¹, Z², Z³, or Z⁴ is a N atom,
A¹, A², A³ are each independently selected from CH, N, NH, O, or S, wherein at least one of A¹, A², A³ is a heteroatom selected from N, O, or S,
R⁴ is selected from halogen, hydroxyl, nitro, cyano, amino, alkyl, alkoxy, aryl, heteroaryl, haloalkyl, haloalkoxy, heterocyclyl, or aryloxy, p is an integer selected from 0, 1, 2, 3, 4, or 5, or two R⁴ form together an aryl, heteroaryl, or heterocyclyl fused to the aromatic ring to which they are attached with p being at least 2, and
R¹, R², R³, R³¹, n, m have the same meaning as that defined hereinabove.

According to a preferred embodiment, the present invention provides compounds of Formula IV, V, VI, VII, VIII, IX, X, XI, or XII, wherein:

Z¹, Z², Z³, Z⁴ are each independently selected from CH or N, wherein at least one of Z¹, Z², Z³, or Z⁴ is a N atom,
A¹, A², A³ are each independently selected from CH, N, NH, O, or S, wherein at least one of A¹, A², A³ is a heteroatom selected from N, O, or S,

R⁴ is selected from halogen, hydroxyl, nitro, cyano, amino, alkyl, alkoxy, aryl, heteroaryl, haloalkyl, haloalkoxy, heterocyclyl, or aryloxy, p is selected from 0, 1, 2, 3, 4, or 5, or two R⁴ form together an aryl, heteroaryl, or heterocyclyl fused to the aromatic ring to which they are attached with p being at least 2,

R¹ is a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
R² is selected from hydrogen, halogen, nitro, cyano, hydroxyl, alkyl, amino, alkoxy, haloalkoxy, aryl, or heteroaryl, m is 0 or 1, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, or 2.

According to a preferred embodiment, the present invention provides compounds having one of the structural Formula XIII, XIV, XV, XVI, XVII, XVIII, XIX, XX, XXI, XXII, XXIII, XXIV, XXV, XXVI, or XXVII, wherein:

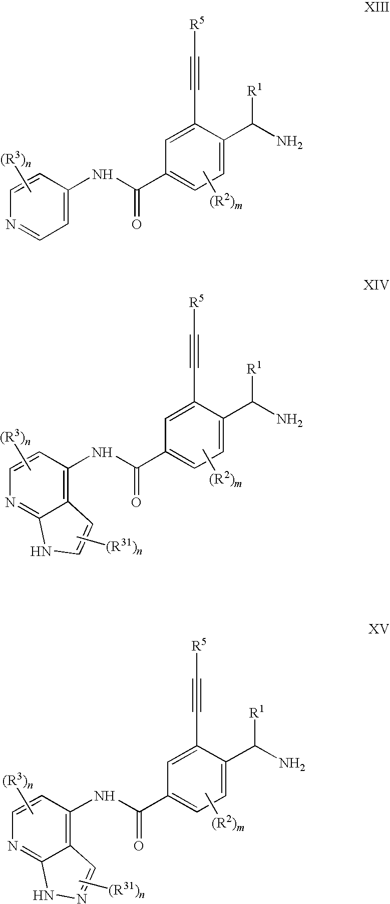

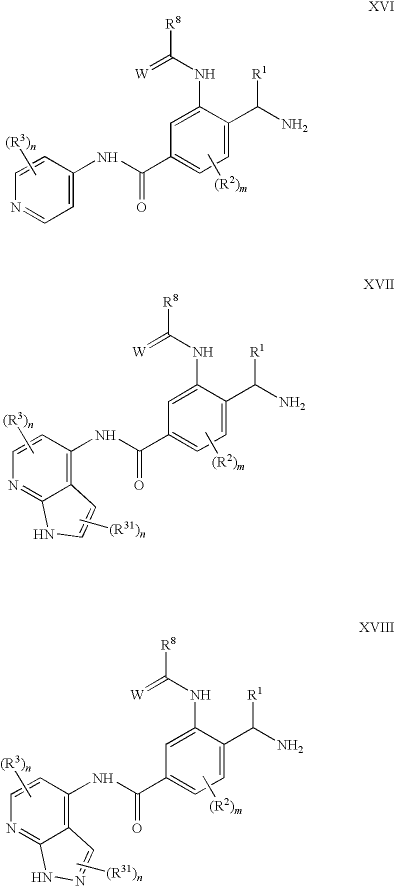

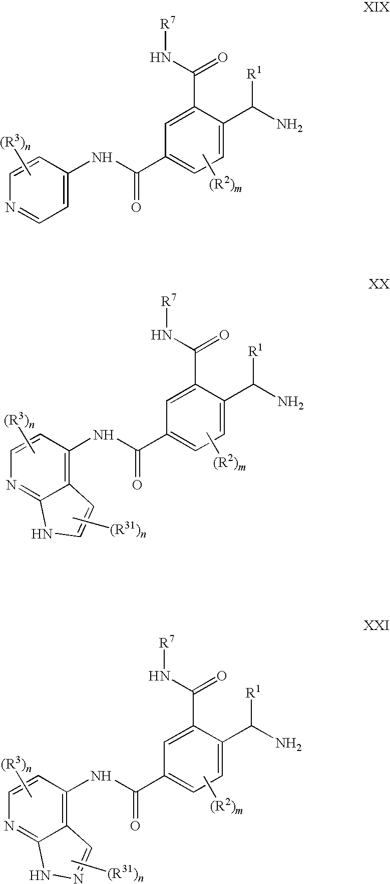

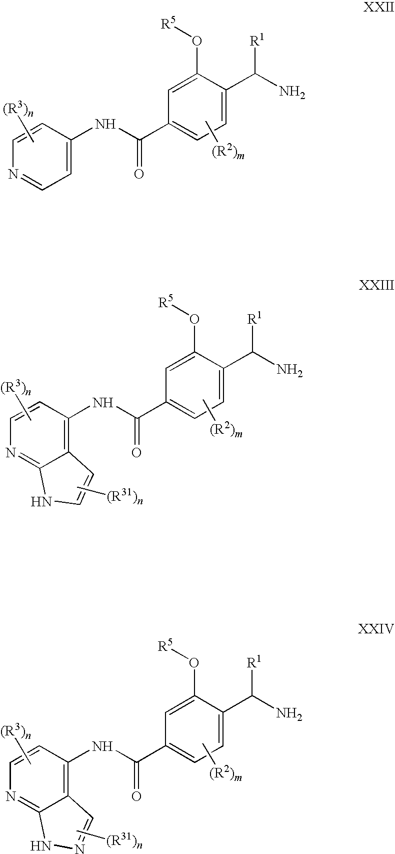

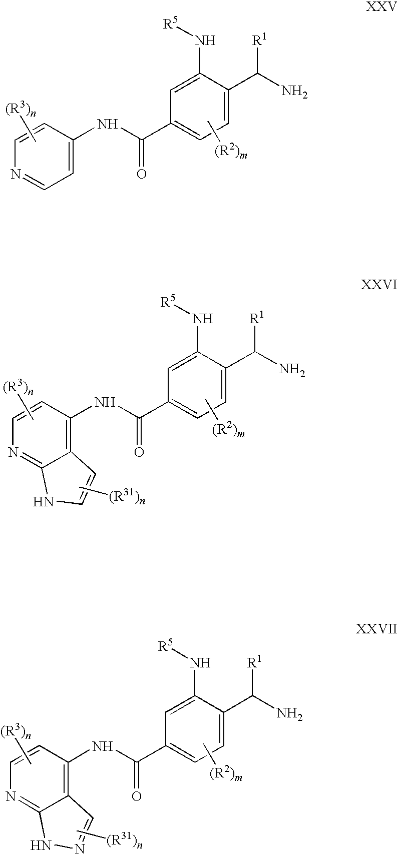

W is selected from O or S,
R⁵ is hydrogen or a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy, and
R¹, R², R³, R³¹, n, m have the same meaning as that defined hereinabove.

According to a preferred embodiment, the present invention provides compounds having one of the structural Formula XIII, XIV, XV, XVI, XVII, XVIII, XIX, XX, XXI, XXII, XXIII, XXIV, XXV, XXVI, or XXVII, wherein:

W is selected from O or S,
R⁵ is hydrogen or a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl, cycloalkyl, heteroaryl, heterocyclyl, each group being optionally substituted by one or more substituents selected from hydroxyl, halogen, oxo, nitro, amino, cyano, alkyl, alkoxy, haloalkyl, or haloalkoxy,
R² is selected from hydrogen, halogen, nitro, cyano, hydroxyl, alkyl, amino, alkoxy, haloalkoxy, aryl, or heteroaryl, and m is 0 or 1, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R⁵, or alkylthio, wherein R¹⁵ is alkyl or cycloalkyl, and n is 0, 1, or 2.

According to a preferred embodiment, the present invention provides compounds having one of the structural Formula XIII, XIV, XV, XVI, XVII, XVIII, XIX, XX, or XXI, wherein:

W is selected from O or S,
R⁵ is hydrogen or a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
m is 0, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1 or 2.

According to another preferred embodiment, the present invention provides compounds having one of the structural Formula XXII, XXIII, XXIV, XXV, XXVI, or XXVII, wherein:

R⁵ is a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl, cycloalkyl, heteroaryl, heterocyclyl, each group being optionally substituted by one or more substituents selected from hydroxyl, halogen, oxo, nitro, amino, cyano, alkyl, alkoxy, haloalkyl, or haloalkoxy,
R² is selected from hydrogen, halogen, nitro, cyano, hydroxyl, alkyl, amino, alkoxy, haloalkoxy, aryl, or heteroaryl, and m is 0 or 1, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl and n is 0, 1, or 2.

More preferably the present invention provides compounds of Formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, or XXVII, wherein

R¹ is a group selected from alkyl or cycloalkyl, each group being optionally substituted by one or two substituents selected from hydroxyl, halogen, oxo, nitro, amino, cyano, alkyl, alkoxy, haloalkyl, or haloalkoxy,
m is 0,
R³ and R³¹ are each independently selected from halogen, hydroxyl, oxo, nitro, cyano, alkyl, and n is 0, 1, or 2,
Z¹, Z², Z³, Z⁴ are each independently selected from CH or N, wherein at least one of Z¹, Z², Z³, or Z⁴ is a N atom,
A¹, A², A³ are each independently selected from CH, N, NH, O, or S, wherein at least one of A¹, A², A³ is a heteroatom selected from N, O, or S,
R⁴ is selected from halogen, hydroxyl, nitro, cyano, amino, alkyl, alkoxy, haloalkyl, haloalkoxy, p is 0, 1, 2, or 3, or two R⁴ form together an aryl, heteroaryl, or heterocyclyl fused to the aromatic ring to which they are attached with p being at least 2, and
W is selected from O or S,
R⁵ is a group selected from aryl, heteroaryl, aralkyl, or heteroarylalkyl, each group being optionally substituted by one or two substituents selected from halogen, hydroxyl, nitro, cyano, amino, alkyl, alkoxy, haloalkyl, haloalkoxy,
R⁶ is —NH—R⁷ or group selected from aryl, heteroaryl, aralkyl, heteroarylalkyl, arylalkylamino, or heteroarylalkylamino, each group being optionally substituted by one or two substituents selected from halogen, hydroxyl, nitro, cyano, amino, alkyl, alkoxy, aryl, heteroaryl, haloalkyl, or haloalkoxy, and
R⁷ is a group selected from optionally substituted: aryl, heteroaryl, heterocyclyl, aralkyl, or heteroarylalky, each group being optionally substituted by one or two substituents selected from halogen, hydroxyl, nitro, cyano, amino, alkyl, alkoxy, aryl, heteroaryl, haloalkyl, or haloalkoxy.

Preferably, the present invention provides a compound of Formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, or XXVII, wherein:

W is selected from O or S,
R⁵ is a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl or cycloalkyl, each group being optionally substituted by one or more substituents selected from hydroxyl, halogen, oxo, nitro, amino, cyano, haloalkyl, or haloalkoxy,
m is 0,
R³ and R³¹ are each independently are selected from halogen, hydroxyl, oxo, nitro, cyano, or alkyl, n is 0, 1, or 2,
Z¹, Z², Z³, Z⁴ are each independently selected from CH or N, wherein at least one of Z¹, Z², Z³, or Z⁴ is a N atom, and
A¹, A², A³ are each independently selected from CH, N, NH, O, or S, wherein at least one of A¹, A², A³ is a heteroatom selected from N, O, or S.

Preferably, the present invention provides a compound of Formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, or XXVII, wherein:

W is selected from O or S,
R⁵ is a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl or cycloalkyl, each group being optionally substituted by one or more substituents selected from hydroxyl, halogen, oxo, nitro, amino, cyano, haloalkyl, or haloalkoxy,
m is 0,
R³ and R³¹ are each independently are selected from halogen, hydroxyl, oxo, nitro, cyano, or alkyl, n is 0, 1, or 2,
Z¹, Z², Z³, Z⁴ are each independently selected from CH or N, wherein at least one of Z¹, Z², Z³, or Z⁴ is a N atom, and
A¹, A², A³ are each independently selected from CH, N, NH, O, or S, wherein at least one of A¹, A², A³ is a heteroatom selected from N, O, or S.

In a particular embodiment, the present invention provides compounds of Formula I, II, or III, wherein

R¹ is a group selected from alkyl or cycloalkyl, each group being optionally substituted by one or more substituents selected from hydroxyl, halogen, oxo, nitro, amino, cyano, haloalkyl, or haloalkoxy,
R² is selected from hydrogen, halogen, nitro, cyano, hydroxyl, alkyl, amino, alkoxy, haloalkoxy, aryl, or heteroaryl, m is 0 or 1,
R³ and R³¹ are each independently are selected from halogen, hydroxyl, oxo, nitro, cyano, or alkyl, n is 0, 1, or 2, and
X is a group selected from hydroxyl, amino, nitro, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, methylamino, ethylamino, n-propylamino, i-propylamino, n-butylamino, i-butylamino, t-butylamino, pentylamino, ethynyl, propynyl, butynyl, pentynyl, hexynyl, methylpropynyl, 4-methyl-1-butynyl, 4-propyl-2-pentynyl, amino-methoxy, 2-amino-ethoxy, 3-amino-propoxy, 4-aminobutoxy, hydroxymethoxy, 2-hydroxy-ethoxy, 3-hydroxy-propoxy, phenyl, biphenylyl, biphenylenyl, 5-tetralinyl, 6-tetralinyl, 1-naphthyl, 2 naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1,2,3,4-tetrahydronaphthyl, 1,4-dihydronaphthyl, 1-pyrenyl, 2-pyrenyl, 3-pyrenyl, 4-pyrenyl, 5-pyrenyl, benzoyl, benzyl, benzoylamino, 3-phenyl-thioureido, 3-phenyl-ureido, 5,8-dihydronaphthalen-1-yl, anilinothiocarbonylamino, benzylamino, benzyloxy, furan-2-ylcarbonylamino, furan-2-ylmethyl-amino, 2-furyl, 3-furyl, 2-thienyl, 3-thienyl, 1-pyrrolyl, 2-pyrrolyl, 3-pyrrolyl, 1-imidazolyl, 2-imidazolyl, 4-imidazolyl, 5-imidazolyl, 1-pyrazolyl, 3-pyrazolyl, 4-pyrazolyl, 5-pyrazolyl, 3-isoxazolyl, 4-isoxazolyl, 5-isoxazolyl, 2-oxazolyl, 4-oxazolyl, 5-oxazolyl, 3-isothiazolyl, 4-isothiazolyl, 5-isothiazolyl, 2-thiazolyl, 4-thiazolyl, 5-thiazolyl, 1,2,3-triazol-1-yl, 1,2,3-triazol-2-yl, 1,2,3-triazol-4-yl, 1,2,3-triazol-5-yl, 1,2,4-triazol-1-yl, 1,2,4-triazol-3-yl, 1,2,4-triazol-4-yl, 1,2,4-triazol-5-yl, 1,2,3-oxadiazol-4-yl, 1,2,3-oxadiazol-5-yl, 1,2,4-oxadiazol-3-, -5-yl, 1,2,5-oxadiazolyl, 1,3,4-oxadiazolyl, 1-tetrazolyl, 5-tetrazolyl, 2-pyridyl, 3-pyridyl, 4-pyridyl, 3-pyridazinyl, 4-pyridazinyl, 2-pyrimidinyl, 4-pyrimidinyl, 5-pyrimidinyl, 6-pyrimidinyl, 2-benzofuryl, 3-benzofuryl, 4-benzofuryl, 5-benzofuryl, 6-benzofuryl, 7-benzofuryl, 2-benzothienyl, 3-benzothienyl, 4-benzothienyl, 5-benzothienyl, 6-benzothienyl, 7-benzothienyl, 1-indolyl, 2-indolyl, 3-indolyl, 4-indolyl, 5-indolyl, 6-indolyl, 7-indolyl, 1,4-oxazin-2-yl, 1,4-oxazin-3-yl, 1,4-dioxin-2-yl, 1,4-dioxin-3-yl, 1,4-thiazin-2-yl, 1,4-thiazin-3-yl, 1,2,3-triazinyl, 1,2,4-triazinyl, 1,3,5-triazin-2-yl, 1,3,5-triazin-4-yl, 1,3,5-triazin-6-yl, 1-benzimidazolyl, 2-benzimidazolyl, 4-benzimidazolyl, 5-benzimidazolyl, 1-benzopyrazolyl, 3-benzopyrazolyl, 4-benzopyrazolyl, 5-benzopyrazolyl, 6-benzopyrazolyl, 7-benzopyrazolyl, 3-benzisoxazolyl, 4-benzisoxazolyl, 5-benzisoxazolyl, 6-benzisoxazolyl, 7-benzisoxazolyl, 2-benzoxazolyl, 4-benzoxazolyl, 5-benzoxazolyl, 6-benzoxazolyl, 7-benzoxazolyl, 3-benzisothiazolyl, 4-benzisothiazolyl, 5-benzisothiazolyl, 6-benzisothiazolyl, 7-benzisothiazolyl, 2-benzothiazolyl, 4-benzothiazolyl, 5-benzothiazolyl, 6-benzothiazolyl, 7-benzothiazolyl, 1-thianthrenyl, 2-thianthrenyl, 3-isobenzofuranyl, 4-isobenzofuranyl, 5-isobenzofuranyl, 2-pyrazinyl, 3-pyrazinyl, 2-isoindolyl, 3-isoindolyl, 4-isoindolyl, 5-isoindolyl, 2-purinyl, 6-purinyl, 7-purinyl, 8-purinyl, 2-quinolyl, 3-quinolyl, 4-quinolyl, 5-quinolyl, 6-quinolyl, 7-quinolyl, 8-quinolyl, 2-quinazolyl, 4-quinazolyl, 5-quinazolyl, 6-quinazolyl, 7-quinazolyl, 8-quinazolyl, 1-isoquinolyl, 3-isoquinolyl, 4-isoquinolyl, 5-isoquinolyl, 6-isoquinolyl, 7-isoquinolyl, 8-isoquinolyl, 3-cinnolinyl, 4-cinnolinyl, 5-cinnolinyl, 6-cinnolinyl, 7-cinnolinyl, 8-cinnolinyl, phenylaminothiocarbonylamino, biphenylylaminothiocarbonylamino, biphenylenylaminothiocarbonylamino, 5-tetralinylaminothiocarbonylamino, 6-tetralinylaminothiocarbonylamino, 1-naphthylaminothiocarbonylamino, 2-naphthylaminothiocarbonylamino, 1-indenylaminothiocarbonylamino, 2-indenylaminothiocarbonylamino, 3-indenylaminothiocarbonylamino, 1-anthrylaminothiocarbonylamino, 2-anthrylaminothiocarbonylamino, 9-anthrylaminothiocarbonylamino, 4-indanylaminothiocarbonylamino, 5-indanylaminothiocarbonylamino, 5-tetrahydronaphthylaminothiocarbonylamino, 6-tetrahydronaphthylaminothiocarbonylamino, 7-tetrahydronaphthylaminothiocarbonylamino, 8-tetrahydronaphthylaminothiocarbonylamino, 1,2,3,4-tetrahydronaphthylaminothiocarbonylamino, 1,4-dihydronaphthylaminothiocarbonylamino, 1-pyrenylaminothiocarbonylamino, 2-pyrenylaminothiocarbonylamino, 3-pyrenylaminothiocarbonylamino, 4-pyrenylaminothiocarbonylamino, 5-pyrenylaminothiocarbonylamino, 2-furylaminothiocarbonylamino, 3-furylaminothiocarbonylamino, 2-thienylaminothiocarbonylamino, 3-thienylaminothiocarbonylamino, 1-pyrrolylaminothiocarbonylamino, 2-pyrrolylaminothiocarbonylamino, 3-pyrrolylaminothiocarbonylamino, 1-pyrazolylaminothiocarbonylamino, 3-pyrazolylaminothiocarbonylamino, 4-pyrazolylaminothiocarbonylamino, 5-pyrazolylaminothiocarbonylamino, 2-thiazolylaminothiocarbonylamino, 4-thiazolylaminothiocarbonylamino, 5-thiazolylaminothiocarbonylamino, 2-pyridylaminothiocarbonylamino, 3-pyridylaminothiocarbonylamino, 4-pyridylaminothiocarbonylamino, 2-pyrimidinylaminothiocarbonylamino, 4-pyrimidinylaminothiocarbonylamino, 5-pyrimidinylaminothiocarbonylamino, 6-pyrimidinylaminothiocarbonylamino, 1-indolylaminothiocarbonylamino, 2-indolylaminothiocarbonylamino, 3-indolylaminothiocarbonylamino, 4-indolylaminothiocarbonylamino, 5-indolylaminothiocarbonylamino, 6-indolylaminothiocarbonylamino, 7-indolylaminothiocarbonylamino, 2-pyrazinylaminothiocarbonylamino, 3-pyrazinylaminothiocarbonylamino, 2-purinylaminothiocarbonylamino, 6-purinylaminothiocarbonylamino, 7-purinylaminothiocarbonylamino, 8-purinylaminothiocarbonylamino, 2-quinolylaminothiocarbonylamino, 3-quinolylaminothiocarbonylamino, 4-quinolylaminothiocarbonylamino, 5-quinolylaminothiocarbonylamino, 6-quinolylaminothiocarbonylamino, 7-quinolylaminothiocarbonylamino, 8-quinolylaminothiocarbonylamino, 2-furylcarbonylamino, 3-furylcarbonylamino, 2-thienylcarbonylamino, 3-thienylcarbonylamino, 1-pyrrolylcarbonylamino, 2-pyrrolylcarbonylamino, 3-pyrrolylcarbonylamino, 1-pyrazolylcarbonylamino, 3-pyrazolylcarbonylamino, 4-pyrazolylcarbonylamino, 5-pyrazolylcarbonylamino, 2-thiazolylcarbonylamino, 4-thiazolylcarbonylamino, 5-thiazolylcarbonylamino, 2-pyridylcarbonylamino, 3-pyridylcarbonylamino, 4-pyridylcarbonylamino, 2-pyrimidinylcarbonylamino, 4-pyrimidinylcarbonylamino, 5-pyrimidinylcarbonylamino, 6-pyrimidinylcarbonylamino, 1-indolylcarbonylamino, 2-indolylcarbonylamino, 3-indolylcarbonylamino, 4-indolylcarbonylamino, 5-indolylcarbonylamino, 6-indolylcarbonylamino, 7-indolylcarbonylamino, 2-pyrazinylcarbonylamino, 3-pyrazinylcarbonylamino, 2-purinylcarbonylamino, 6-purinylcarbonylamino, 7-purinylcarbonylamino, 8-purinylcarbonylamino, 2-quinolylcarbonylamino, 3-quinolylcarbonylamino, 4-quinolylcarbonylamino, 5-quinolylcarbonylamino, 6-quinolylcarbonylamino, 7-quinolylcarbonylamino, 8-quinolylcarbonylamino, phenylcarbonylamino, biphenylylcarbonylamino, biphenylenylcarbonylamino, 1-naphthylcarbonylamino, 2-naphthylcarbonylamino, 1-indenylcarbonylamino, 2-indenylcarbonylamino, 3-indenylcarbonylamino, 4-indanylcarbonylamino, 5-indanylcarbonylamino, 5-tetrahydronaphthylcarbonylamino, 6-tetrahydronaphthylcarbonylamino, 7-tetrahydronaphthylcarbonylamino, 8-tetrahydronaphthylcarbonylamino, 1,2,3,4-tetrahydronaphthylcarbonylamino, 1,4-dihydronaphthylcarbonylamino, 1-pyrenylcarbonylamino, 2-pyrenylcarbonylamino, 3-pyrenylcarbonylamino, 4-pyrenylcarbonylamino, or 5-pyrenylcarbonylamino,
each group being optionally substituted by one, two, or three substituents selected from methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloro, fluoro, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, hydroxyl, oxo, nitro, cyano, amino, aminocarbonyl, carboxyl, phenyl, 1-naphthyl, 2-naphthyl, 2-thienyl, 3-thienyl, 2-furyl, 3-furyl, hydroxymethyl, 2-hydroxyethyl, phenyloxy, biphenylyloxy, biphenylenyloxy, 1-naphthyloxy, 2-naphthyloxy, methylamino, ethylamino, n-propylamino, i-propylamino, n-butylamino, i-butylamino, t-butylamino, pentylamino, or hexylamino.

In a preferred embodiment, the compounds of the invention are of formula I, II, or III, wherein

R¹ is a group selected from alkyl or cycloalkyl, each group being optionally substituted by one or more substituents selected from hydroxyl, halogen, oxo, nitro, amino, cyano, haloalkyl, or haloalkoxy,
m is 0,
R³ and R³¹ are each independently are selected from halogen, hydroxyl, oxo, nitro, cyano, or alkyl, n is 0, 1, or 2, and
X is a group selected from hydroxyl, amino, nitro, ethynyl, propynyl, butynyl, pentynyl, hexynyl, methylpropynyl, 4-methyl-1-butynyl, 4-propyl-2-pentynyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, amino-methoxy, 2-amino-ethoxy, 3-amino-propoxy, 4-aminobutoxy, 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-thiazolyl, 4-thiazolyl, 5-thiazolyl, 2-pyridyl, 3-pyridyl, 4-pyridyl, 2-pyrimidinyl, 4-pyrimidinyl, 5-pyrimidinyl, 6-pyrimidinyl, 1-indolyl, 2-indolyl, 3-indolyl, 4-indolyl, 5-indolyl, 6-indolyl, 7-indolyl, 2-pyrazinyl, 3-pyrazinyl, 2-purinyl, 6-purinyl, 7-purinyl, 8-purinyl, 2-quinolyl, 3-quinolyl, 4-quinolyl, 5-quinolyl, 6-quinolyl, 7-quinolyl, 8-quinolyl, hydroxymethoxy, 2-hydroxy-ethoxy, 3-hydroxy-propoxy, phenyl, biphenylyl, biphenylenyl, 1-naphthyl, 2-naphthyl, 1-indenyl, 2-indenyl, 3-indenyl, 4-indanyl, 5-indanyl, 5-tetrahydronaphthyl, 6-tetrahydronaphthyl, 7-tetrahydronaphthyl, 8-tetrahydronaphthyl, 1,2,3,4-tetrahydronaphthyl, 1,4-dihydronaphthyl, 1-pyrenyl, 2-pyrenyl, 3-pyrenyl, 4-pyrenyl, 5-pyrenyl, 3,4-dichloro-phenyl, 3-chloro-benzoylamino, 2′-chloro-phenyl, 2′-methoxy-phenyl, 3-chloro-phenyl, 3-cyano-phenyl, 3-methoxy-benzoylamino, 3-methoxy-phenyl, 3-phenyl-thioureido, 3-phenyl-ureido, 4-chloro-benzoylamino, 4-chloro-phenyl, 4-cyano-phenyl, 4-fluoro-phenyl, 4-hydroxymethyl-phenyl, 4-hydroxy-phenyl, 4-methoxy-benzoylamino, 4-methoxy-phenyl, 5,8-dihydronaphthalen-1-yl, anilinothiocarbonylamino, benzoylamino, benzylamino, benzyloxy, furan-2-ylcarbonylamino, furan-2-ylmethyl-amino, phenylaminothiocarbonylamino, biphenylylaminothiocarbonylamino, biphenylenylaminothiocarbonylamino, 1-naphthylaminothiocarbonylamino, 2-naphthylaminothiocarbonylamino 1-indenylaminothiocarbonylamino, 2-indenylaminothiocarbonylamino, 3-indenylaminothiocarbonylamino, 4-indanylaminothiocarbonylamino, 5-indanylaminothiocarbonylamino, 5-tetrahydronaphthylaminothiocarbonylamino, 6-tetrahydronaphthylaminothiocarbonylamino, 7-tetrahydronaphthylaminothiocarbonylamino, 8-tetrahydronaphthylaminothiocarbonylamino, 1,2,3,4-tetrahydronaphthylaminothiocarbonylamino, 1,4-dihydronaphthylaminothiocarbonylamino, 2-furylaminothiocarbonylamino, 3-furylaminothiocarbonylamino, 2-thienylaminothiocarbonylamino, 3-thienylaminothiocarbonylamino, 1-pyrrolylaminothiocarbonylamino, 2-pyrrolylaminothiocarbonylamino, 3-pyrrolylaminothiocarbonylamino, 1-pyrazolylaminothiocarbonylamino, 3-pyrazolylaminothiocarbonylamino, 4-pyrazolylaminothiocarbonylamino, 5-pyrazolylaminothiocarbonylamino, 2-pyridylaminothiocarbonylamino, 3-pyridylaminothiocarbonylamino, 4-pyridylaminothiocarbonylamino, 2-pyrimidinylaminothiocarbonylamino, 4-pyrimidinylaminothiocarbonylamino, 5-pyrimidinylaminothiocarbonylamino, 6-pyrimidinylaminothiocarbonylamino, 1-indolylaminothiocarbonylamino, 2-indolylaminothiocarbonylamino, 3-indolylaminothiocarbonylamino, 4-indolylaminothiocarbonylamino, 5-indolylaminothiocarbonylamino, 6-indolylaminothiocarbonylamino, 7-indolylaminothiocarbonylamino, 2-pyrazinylaminothiocarbonylamino, 3-pyrazinylaminothiocarbonylamino, 2-purinylaminothiocarbonylamino, 6-purinylaminothiocarbonylamino, 7-purinylaminothiocarbonylamino, 8-purinylaminothiocarbonylamino, 2-furylcarbonylamino, 3-furylcarbonylamino, 2-thienylcarbonylamino, 3-thienylcarbonylamino, 1-pyrrolylcarbonylamino, 2-pyrrolylcarbonylamino, 3-pyrrolylcarbonylamino, 1-pyrazolylcarbonylamino, 3-pyrazolylcarbonylamino, 4-pyrazolylcarbonylamino, 5-pyrazolylcarbonylamino, 2-pyridylcarbonylamino, 3-pyridylcarbonylamino, 4-pyridylcarbonylamino, 2-pyrimidinylcarbonylamino, 4-pyrimidinylcarbonylamino, 5-pyrimidinylcarbonylamino, 6-pyrimidinylcarbonylamino, 1-indolylcarbonylamino, 2-indolylcarbonylamino, 3-indolylcarbonylamino, 4-indolylcarbonylamino, 5-indolylcarbonylamino, 6-indolylcarbonylamino, 7-indolylcarbonylamino, 2-pyrazinylcarbonylamino, 3-pyrazinylcarbonylamino, 2-purinylcarbonylamino, 6-purinylcarbonylamino, 7-purinylcarbonylamino, 8-purinylcarbonylamino, phenylcarbonylamino, biphenylylcarbonylamino, biphenylenylcarbonylamino, 1-naphthylcarbonylamino, 2-naphthylcarbonylamino, 1-indenylcarbonylamino, 2-indenylcarbonylamino, 3-indenylcarbonylamino, 4-indanylcarbonylamino, 5-indanylcarbonylamino, 5-tetrahydronaphthylcarbonylamino, 6-tetrahydronaphthylcarbonylamino, 7-tetrahydronaphthylcarbonylamino, 8-tetrahydronaphthylcarbonylamino, 1,2,3,4-tetrahydronaphthylcarbonylamino, 1,4-dihydronaphthylcarbonylamino, each group being optionally substituted by one or two substituents selected from methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloro, fluoro, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, oxohydroxyl, nitro, cyano, amino, aminocarbonyl, phenyl, 1-naphthyl, 2-naphthyl, 2-thienyl, 3-thienyl, 2-furyl, 3-furyl, hydroxymethyl, 2-hydroxyethyl, phenyloxy, biphenylyloxy, biphenylenyloxy, 1-naphthyloxy, or 2-naphthyloxy.

The term “stereoisomer” as used herein, defines all possible compounds made up of the same atoms bonded by the same sequence of bonds but having different three-dimensional structures which are not interchangeable, which the compounds of the present invention may possess. It will be clear to the skilled person that some of the compounds of the invention may contain one or more asymmetric carbon atoms that serve as a chiral center, which may lead to different optical forms (e.g. enantiomers or diastereoisomers). Unless otherwise mentioned or indicated, the chemical designation of a compound herein encompasses all such optical forms in all possible configurations as well as the mixture of all possible stereochemically isomeric forms, which said compound may possess. Said mixture may contain all diastereomers and/or enantiomers of the basic molecular structure of said compound. All stereochemically isomeric forms of the compounds of the invention either in pure form or in admixture with each other are intended to fall within the scope of the present invention. This asymmetric center is indicated with an asterisk (*) in the figure below.

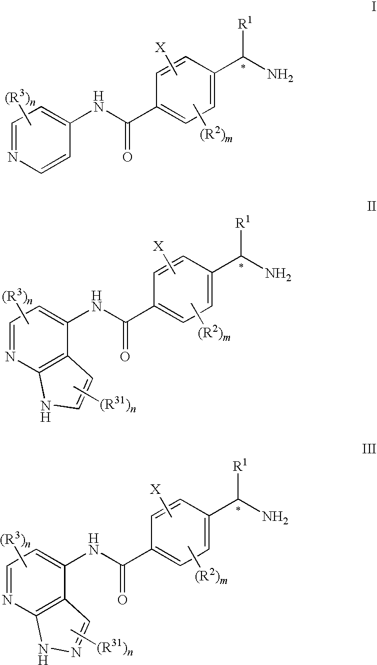

The compounds of the invention may be prepared as described in the experimental section below using methods and chemistries with which those skilled in the art shall be familiar.

According to a particular embodiment, preferably when R¹ is selected from optionally substituted alkyl or cycloalkyl as defined above, the present invention encompasses the method for the preparation of enantiomers of Formula I(R), I(S), II(R), II(S), III(R) and III(S).

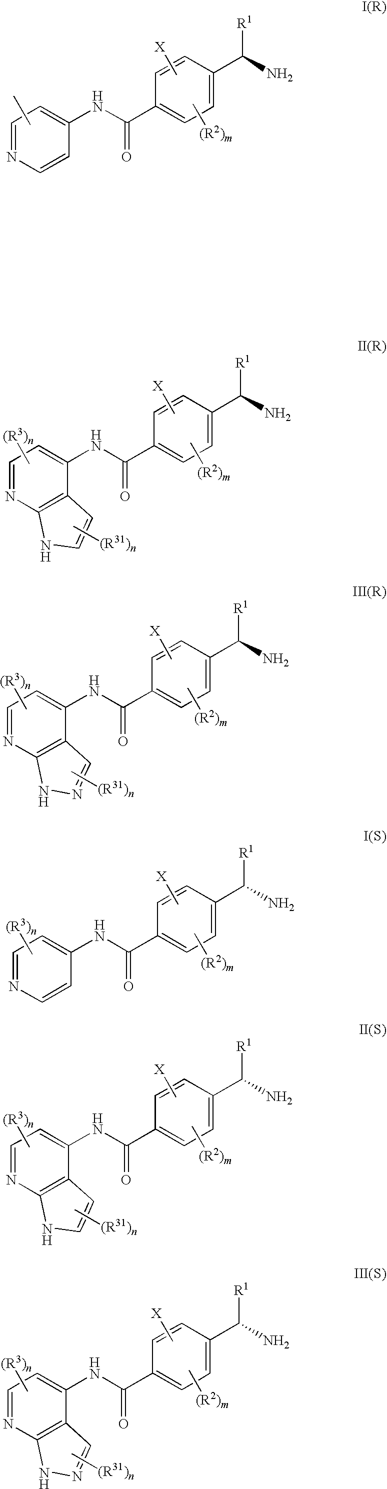

For example, enantiomers of Formula I (I(R) or I(S)) can be obtained: by reacting a compound of Formula XXVIII with Noyori's catalyst (JACS, 1996, 118, 2521; JACS, 2005, 127, 4596), thereby obtaining compounds of Formula XXIX(R) or XXIX(S).

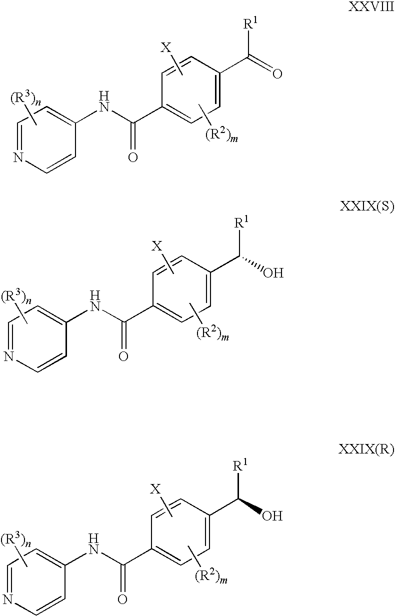

Noyori's catalyst can be obtained by reacting dichloro(p-cymene)ruthenium (II) dimer (0.05 eq.) with (1S,2S)-(+)-N-p-tosyl-1,2-diphenylethylenediamine or (1R,2R)-(+)-N-p-tosyl-1,2-diphenylethylenediamine.

Compound of Formula XXIX (configuration R or S) is then reacted with diphenylphosphoryl azide (DPPA) and with 1,8-diazabicyclo[5.4.0]undec-7-ene (DBU) to give azide of Formula XXX (configuration R or S).

The azide of Formula XXX is then reacted with Pd/C to give the amine of Formula I(R) or I(S).

For example, according to the protocol illustrated in scheme 1, compounds of Formula I(R) can be obtained from compound of Formula XXVIII.

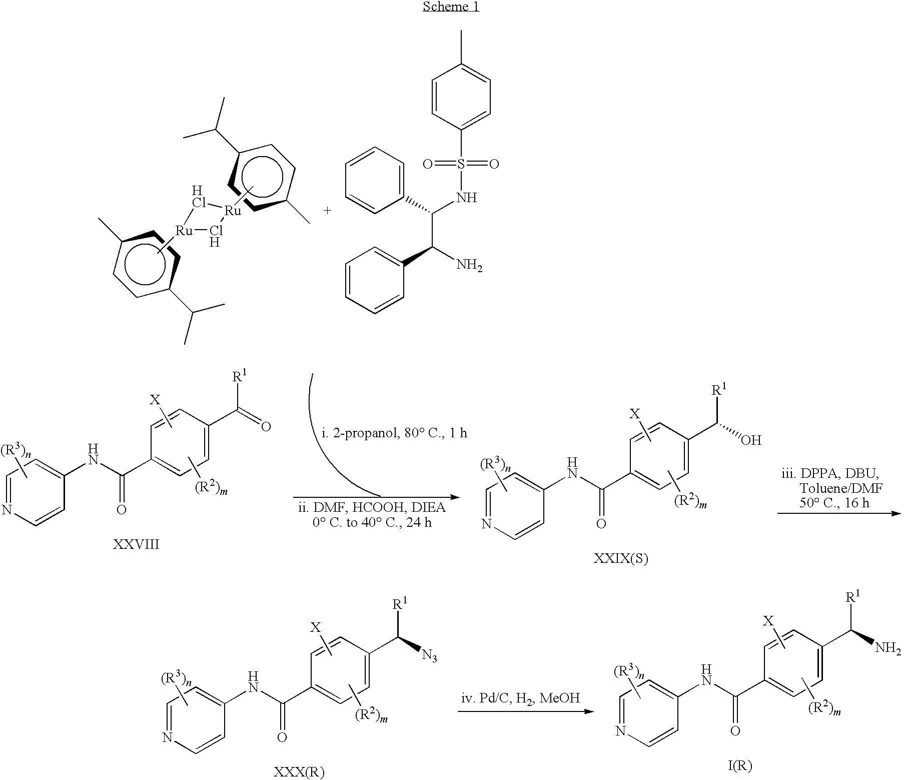

More generally, from the above, it will be clear to the skilled person that some of the compounds of the invention may exist in the form of different isomers and/or tautomers, including but not limited to geometrical isomers, conformational isomers, and stereochemical isomers (i.e. enantiomers and diastereoisomers) and isomers that correspond to the presence of the same substituents on different positions of the rings present in the compounds of the invention. All such possible isomers, tautomers and mixtures thereof are included within the scope of the invention.

According to a particular embodiment, the present invention encompasses the method for the preparation of compound of Formula II.

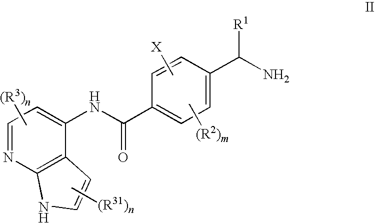

Compound II can be obtained by reaction a compound of Formula XXXI, wherein PG means a suitable protective group, such as but not limited to TIPS [tri-isopropylsilyl], or benzyloxycarbonyl, with a compound of Formula XXXII or XXXIII, wherein PG has the same meaning as that defined above, such as t-butyloxycarbonyl for example.

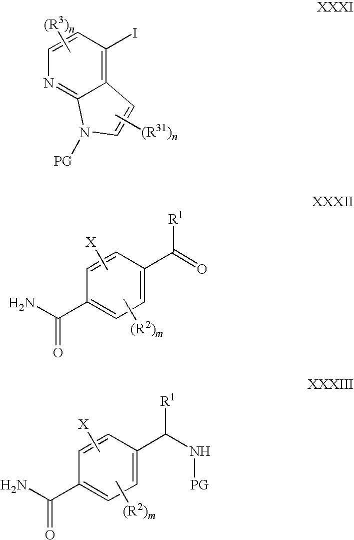

For example, according to the protocol illustrated in schemes 2 and 3, compounds of Formula II can be obtained from compound of Formula XXXI and XXXII or XXXIII.

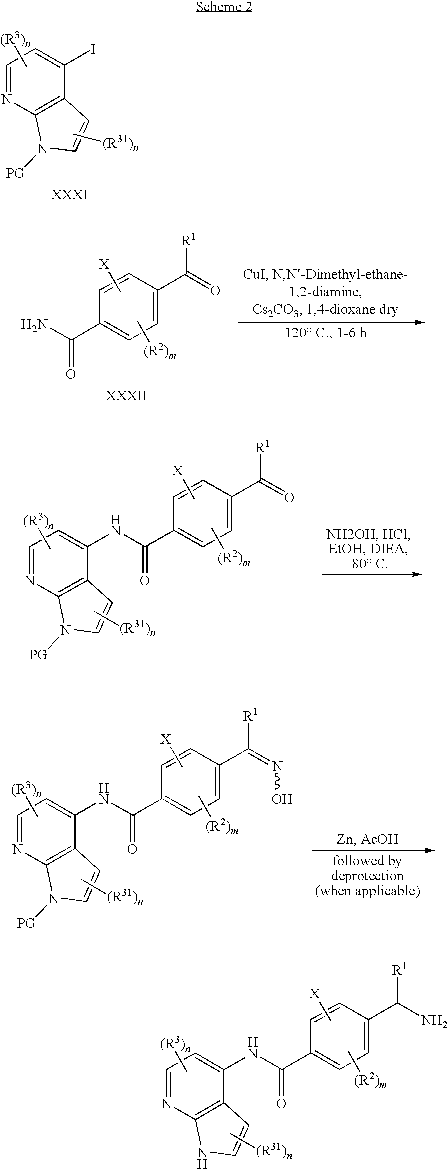

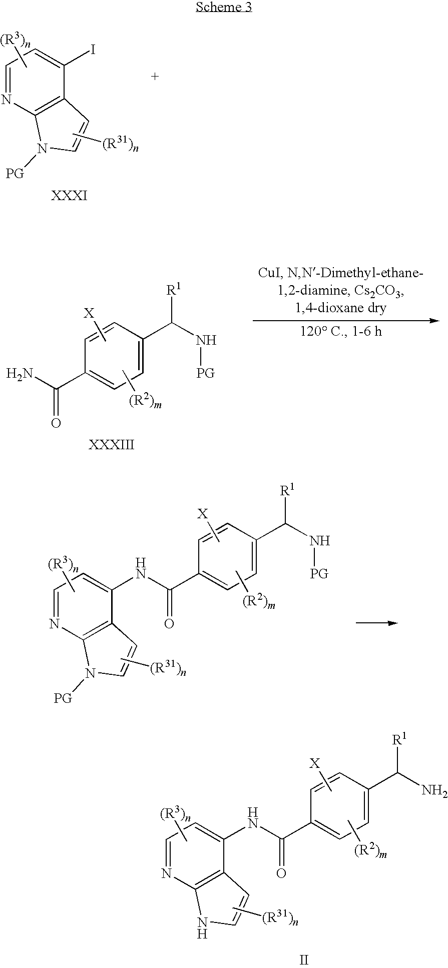

It will also be clear that when the desired compounds of the invention, and/or the starting materials, precursors and/or intermediates used in the preparation thereof, contain functional groups that are sensitive to the reaction conditions used in the preparation of the compounds of the invention (i.e. that would undergo undesired reactions under those conditions if they were not suitably protected) can be protected during said reaction with one or more suitable protective group, which protective group can then be suitably removed after either completion of said reaction and/or as a later or final step in the preparation of the compounds of the invention. Protected forms of the inventive compounds are included within the scope of the present invention. Suitable protective groups, as well as methods and conditions for inserting them and removing them, will be clear to the skilled person and are generally described in the standard handbooks of organic chemistry, such as Greene and Wuts, “Protective groups in organic synthesis”, 3^{rd} Edition, Wiley and Sons, 1999, which is incorporated herein by reference in its entirety. It will also be clear to the skilled person that compounds of the invention in which one or more functional groups have been protected with suitable functional groups can find use as intermediates in the production and/or synthesis of the compounds of the invention, and as such form a further aspect of the invention.

Generally, the compounds of the invention are prepared from intermediates 1, 2, 3, 4, or 5 described hereinafter which may be reacted with complementary reactive molecules so as to form the desired compound.

The compounds of the invention may be in the form of pharmaceutically and/or veterinary acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid, and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I, II, and III above.

The invention also generally covers all pharmaceutically acceptable predrugs and prodrugs of the compounds of Formula I, II, III, for which general reference is made to the prior art cited hereinbelow.

The term “pro-drug” as used herein means the pharmacologically acceptable derivatives such as esters, amides, and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component.

Typical examples of pro-drugs are described for instance in WO 99/33795, WO 99/33815, WO 99/33793, and WO 99/33792 all incorporated herein by reference. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

As described, above, some of the compounds of the invention may contain one or more asymmetric carbon atoms that serve as a chiral center, which may lead to different optical forms (e.g. enantiomers or diastereoisomers). The invention comprises all such optical forms in all possible configurations, as well as mixtures thereof.

More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substituents on different positions of the rings present in the compounds of the invention. All such possible isomers, tautomers, and mixtures thereof are included within the scope of the invention.

The compounds of the invention may be used for the inhibition of kinases in vitro or in vivo, preferably in vitro, for modulating biological pathways and/or processes in which such kinases are involved; and/or to prevent and/or treat diseases or disorders in which such kinases, pathways and/or processes are involved.

According to one preferred, but non-limiting embodiment, the compounds of the invention may be used to inhibit (at least one isoform of) ROCK; and as such may be used for any purposes known per se for inhibitors of ROCK.

In the invention, particular preference is given to compounds of Formula I, II, III above that in the inhibition assay for ROCK described below inhibit ROCK with an IC₅₀ value of less than 100 μM, preferably less than 50 μM, more preferably less than 10 μM, preferably less than 5 μM, even more preferably less than 1 μM preferably less than 0.1 μM, and in particular less than 10 nM, for example less than 1 nM, as determined by a suitable assay, such as the assay used in the Examples below.

The present invention also relates to the use of the compounds of Formula I, II, III above in (the preparation of a composition for) inhibiting at least one kinase, in particular for inhibiting at least one isoform of ROCK, more in particular for inhibiting ROCK I and/or ROCK II isoforms. As used herein, the term “ROCKI” can also be referred as ROK-β, p160ROCK, or Rho-kinase β, and the term “ROCKII” can also be referred as ROK-α or Rho-kinase α.

Said inhibition may be effected in vitro and/or in vivo, and when effected in vivo, is preferably effected in a selective manner, as defined above.

According to an embodiment, the invention provides a method for treating or lessening the severity of a ROCK-mediated disease or condition in a patient comprising the step of administering to said patient a compound according to the present invention.

The term “ROCK-mediated condition” or “disease”, as used herein, means any disease or other deleterious condition in which is known to play a role. The term “ROCK-mediated condition” or “disease” also means those diseases or conditions that are alleviated by treatment with a ROCK inhibitor. Accordingly, another embodiment of the present invention relates to treating or lessening the severity of one or more diseases in which ROCK is known to play a role.

According to particularly preferred embodiments, the compounds of the invention are preferably used in the prevention and/or treatment of at least one disease or disorder, preferably in which at least one isoform of ROCK is involved. According to an even more particularly preferred embodiment, the compounds of the invention may be used in the prevention and/or treatment of at least one disease or disorder in which the ROCK I or ROCK II is involved, such as, such as inflammatory diseases, chronic obstructive bladder disease (COBD), and the related erectile dysfunction as well as in diabetes related ED.

Specifically, the present invention relates to the use of a compound according to the invention for the preparation of a medicament for treating or lessening the severity of a disease or condition selected from eye disease or disorder (such as but not limited to retinopathy, glaucoma, and degenerative retinal diseases such as macular degeneration, and retinitis pigmentosa), kidney disease (such as but not limited to renal dysfunction), and bladder dysfunction (such as but not limited to chronic obstructive bladder disease), erectile dysfunction (such as but not limited to bladder disease related erectile dysfunction, and diabetes related erectile dysfunction) neurological, and CNS (brain) disease or disorder (such as but not limited to Alzheimer, meningitis, and convulsions), hypertension, lung disease (such as but not limited to asthma, fibrosis, pneumonia, cystic fibrosis, and respiratory distress syndrome), premature birth, cancer (such as but not limited to cancer of the brain (gliomas), breast, colon, head and neck, prostate, kidney, lung, intestine, nerve, skin, pancreas, liver, uterus, ovary, brain, thyroid gland; leukemia; lymphoma, and melanoma), cardiovascular and vascular (blood vessel, artery) disease or disorder (such as but not limited to cerebrovascular contraction, ischemia, reperfusion, pulmonary vasoconstriction, acute stroke, congestive heart failure, cardiovascular ischemia, heart disease, cardiac remodeling, hypoxia peripheral circulation disorder, atherosclerosis, thrombosis, aneurism, and hemorrhage), blood disease (such as but not limited to sepsis, eosinophilia, endotoxemia), musculoskeletal disease (such as but not limited to spasm), inflammatory disease, infection, allergy, and autoimmune diseases or disorders, AIDS, bone disease (such as but not limited to osteoporosis), inflammatory diseases, diabetes (such as but not limited to hyperglycemia), obesity, and pancreas disease.

For example, the compounds of the invention may be used in the prevention and/or treatment of diseases and disorders such as:

Cardiovascular and vascular diseases: including but not limited to acute stroke, congestive heart failure, cardiovascular ischemia, heart disease, cardiac remodeling, angina, coronary vasospasm, cerebral vasospasm, restenosis, hypertension, (pulmonary) hypertension, pulmonary vasoconstriction, arteriosclerosis, thrombosis (including deep thrombosis), and platelet related diseases.

Neurological and CNS disorders: including but not limited to stroke, multiple sclerosis, brain or spinal cord injury, inflammatory, and demyelinating diseases such as Alzheimer's disease, MS, and neuropathic pain. The present compounds are therefore suitable for preventing neurodegeneration and stimulating neurogeneration in various neurological disorders.

Proliferative diseases: such as cancer including but not limited to cancer of the brain (gliomas), breast, colon, intestine, skin, head and neck, kidney, lung, liver, ovarian, pancreatic, prostate, or thyroid; leukemia; sarcoma; lymphoma; and melanoma.

Inflammatory diseases: including but not limited to contact dermatitis, psoriasis, rheumatoid arthritis, inflammatory bowel disease, Crohn's disease, and ulcerative colitis. Preferably, the compound may be used in (the preparation of a medicament for) the prevention and/or treatment of Inflammatory diseases selected from contact dermatitis, psoriasis, rheumatoid arthritis, inflammatory bowel disease, Crohn's disease and ulcerative colitis and/or for preventing, treating and/or alleviating complications and/or symptoms associated therewith.

In addition, the compounds of the invention may be used in the prevention and/or treatment of diseases and disorders such as erectile dysfunction; bronchial asthma; osteoporosis; renal diseases; AIDS; eye diseases such as glaucoma, macular degeneration and retinopathy. Preferably, the compound may be used in the prevention and/or treatment of glaucoma and/or for preventing, treating and/or alleviating complications and/or symptoms associated therewith.

The present invention therefore relates to a method of treating or lessening the severity of a disease or condition selected from cardiovascular and vascular diseases including but not limited to acute stroke, congestive heart failure, cardiovascular ischemia, heart disease, cardiac remodeling, angina, coronary vasospasm, cerebral vasospasm, pulmonary vasoconstriction, restenosis, hypertension, (pulmonary) hypertension, arteriosclerosis, thrombosis (including deep thrombosis), and platelet related diseases; neurological, and CNS disorders: including but not limited to stroke, multiple sclerosis, brain or spinal cord injury, inflammatory, and demyelinating diseases such as Alzheimer's disease, MS, and neuropathic pain; proliferative diseases such as cancer including but not limited to cancer of the brain (gliomas), breast, colon, intestine, skin, head and neck, kidney, lung, liver, ovarian, pancreatic, prostate, or thyroid; leukemia; sarcoma; lymphoma; melanoma; erectile dysfunction; bronchial asthma; osteoporosis; eye diseases such as glaucoma, macular degeneration, and retinopathy; renal diseases; AIDS; preterm labor; vascular smooth muscle cell proliferation; myocardial hypertrophy; malignoma; ischemia/reperfusion-induced injury; endothelial dysfunction, Crohn's Disease, and colitis; neurite outgrowth; Raynaud's Disease; benign prostatic hyperplasia; and atherosclerosis, wherein said method comprises administering to a patient in need thereof a compound or a composition according to the present invention.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters, and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087, and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides, and iodides; dialkyl sulfates like dimethyl, diethyl, dibutyl; and diamyl sulfates, long chain halides such as decyl, lauryl, myristyl, and stearyl chlorides, bromides, and iodides, aralkyl halides like benzyl and phenethyl-bromides and others. Other pharmaceutically acceptable salts include the sulfate salt ethanolate and sulfate salts.

Generally, for pharmaceutical use, the compounds of the inventions may be formulated as a pharmaceutical preparation comprising at least one compound of the invention and at least one pharmaceutically acceptable carrier, diluent, or excipient and/or adjuvant, and optionally one or more further pharmaceutically active compounds.

By means of non-limiting examples, such a formulation may be in a form suitable for oral administration, for parenteral administration (such as by intravenous, intramuscular, or subcutaneous injection, or intravenous infusion), for topical administration (including ocular), for administration by inhalation, by a skin patch, by an implant, by a suppository, etc. Such suitable administration forms—which may be solid, semi-solid, or liquid, depending on the manner of administration—as well as methods and carriers, diluents, and excipients for use in the preparation thereof, will be clear to the skilled person; reference is again made to for instance U.S. Pat. No. 6,372,778, U.S. Pat. No. 6,369,086, U.S. Pat. No. 6,369,087, and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions, and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying, and suspending agents, dispersing agents, desintegrants, bulking agents, fillers, preserving agents, sweetening agents, flavoring agents, flow regulators, release agents, etc. The compositions may also be formulated so as to provide rapid, sustained, or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β-, or γ-cyclodextrins or their derivatives. In addition, co-solvents such as alcohols may improve the solubility and/or the stability of the compounds. In the preparation of aqueous compositions, addition of salts of the compounds of the invention can be more suitable due to their increased water solubility.

Appropriate cyclodextrins are α-, β-, or γ-cyclodextrins (CDs) or ethers and mixed ethers thereof wherein one or more of the hydroxy groups of the anhydroglucose units of the cyclodextrin are substituted with alkyl, particularly methyl, ethyl, or isopropyl, e.g. randomly methylated β-CD; hydroxyalkyl, particularly hydroxyethyl, hydroxypropyl, or hydroxybutyl; carboxyalkyl, particularly carboxymethyl or carboxyethyl; alkylcarbonyl, particularly acetyl; alkoxycarbonylalkyl or carboxyalkoxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD, and (2-carboxymethoxy)propyl-β-CD, and in particular 2-hydroxypropyl-β-CD (2-HP-β-CD). The term mixed ether denotes cyclodextrin derivatives wherein at least two cyclodextrin hydroxy groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors. In particular, the present invention encompasses a pharmaceutical composition comprising an effective amount of a compound according to the invention with a pharmaceutically acceptable cyclodextrin. The present invention also encompasses cyclodextrin complexes consisting of a compound according to the invention and a cyclodextrin.

Particular reference is made to the compositions, formulations (and carriers, excipients, diluents, etc. for use therein), routes of administration etc., which are known per se for analogous pyridinocarboxamides, such as those described in U.S. Pat. No. 4,997,834, and EP-A-0 370 498.

For the treatment of pain, the compounds of the invention may be used locally or systemically. For local administration, the compounds may advantageously be used in the form of a spray, ointment or transdermal patch or another suitable form for topical, transdermal and/or intradermal administration; and for systemic administration, the compounds of the invention may advantageously be administered orally.

For ophthalmic application, solutions, gels, tablets, and the like are often prepared using a physiological saline solution, gel or excipient as a major vehicle. Ophthalmic formulations should preferably be prepared at a comfortable pH with an appropriate buffer system.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products, and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded, or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides, and derivatives thereof.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087, and U.S. Pat. No. 6,372,733, and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule, or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300, or 400 mg per unit dosage.

The compounds can be administered by a variety of routes including the oral, rectal, ocular, transdermal, subcutaneous, intravenous, intramuscular, or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formula I, II, or III above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200, or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778,U.S. Pat. No. 6,369,086, U.S. Pat. No. 6,369,087, and U.S. Pat. No. 6,372,733, and the further prior art mentioned above, as well as to the standard handbooks, such as the latest edition of Remington's Pharmaceutical Sciences.

Thus, in a further aspect, the invention relates to a composition, and in particular a composition for pharmaceutical use, that contains at least one compound of the invention (i.e. a compound that has been identified, discovered and/or developed using a nematode or method as described herein) and at least one suitable carrier (i.e. a carrier suitable for pharmaceutical use). The invention also relates to the use of a compound of the invention in the preparation of such a composition.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate, and lactose and/or other excipients, binders, extenders, disintegrants, diluents, and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions, or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers, and stabilizers as well as a propellant.

For subcutaneous or intravenous administration, the compound according to the invention, if desired with the substances customary therefore such as solubilizers, emulsifiers, or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters, or polyethylene glycols, which are solid at ordinary temperatures, but liquefy and/or dissolve in the rectal cavity to release the drug.

The compositions are also of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention and at least one suitable carrier (i.e. a carrier suitable for veterinary use). The invention also relates to the use of a compound of the invention in the preparation of such a composition.

The invention will now be illustrated by means of the following synthetic and biological examples, which do not limited the scope of the invention in any way.

### EXAMPLES

The invention will now be illustrated by means of the following synthetic and biological examples, which do not limited the scope of the invention in any way.

Unless indicated otherwise, the purity of the compounds was confirmed by liquid chromatography/mass spectrometry (LC/MS), as follows:
HPLC system: Waters 2690 with photodiode array detector Waters 996; Column: C18; Gradient: solvent A (H₂O/formic acid 26.5 nM) 0%, to solvent B (CH₃CN/formic acid 17 nM) 80% in 3 min. Flow: 2.75 ml/min.
Mass spectrometer: Micromass Platform LC. Ionization: electrospray (polarity: negative and positive).

Purification of compounds using preparative HPLC was done as follows:

HPLC system: Shimadzu SCL-10A with a detector Shimadzu SPD-10A

Column: 20×200 mm, C18 phase, Nucleosil (100 Å, 100 μm)

Solvent A: H₂O/formic acid 26.5 nM; solvent B: CH₃CN/formic acid 17 nM

NMR spectra were determined on a Varian Mercury 300 MHz NMR using the indicated solvent as an internal reference. Melting points were determined on a Büchi B-540 and are non-corrected. All reagents used either were obtained commercially or were prepared in a manner known per se.

Extra analytical (or preparative) techniques:

Unless indicated otherwise, purification by preparative HPLC, was performed on a Shimadzu SCL-10A (UV detection at 215 and 254 nm, detector SPD-10A) using C-18 column (Nucleosil, 100A, 100 μm, 20×200 mm) and different gradients (water, acetonitrile, formic acid).

Chiral HPLC (analytical and preparative) was performed on a Shimadzu SCL-10A (UV detection at 215 and 254 nm, detector SPD-10A) using different column such as Chiralcel OD-H (tris-3,5-dimethylphenylcarbamate, 46×250 or 100×250 mm, 5 μm), Chiralcel OJ (tris-methylbenzoate, 46×250 or 100×250 mm, 5 μm), Chiralpak AD (tris-3,5-dimethylphenylcarbamate, 46×250 mm, 10 μm) and Chiralpak AS (tris-(S)-1-phenylethylcarbamate, 46×250 mm, 10 μm) from Chiral Technologies Europe (Ilikirch, France):
Eluent: mixture of solvent such as ethanol, 1-propanol, 2-propanol, methanol, butanol, pentane, hexane, heptane, cyclohexane, diisopropylethylamine, triethylamine.
Flow: between 1 and 50 ml/min.

### Example 1

### Intermediates

The following intermediates and general procedures were used to prepare the compounds described herein.

### Intermediate 1: 4-acetyl-3-hydroxy-N-pyridin-4-yl-benzamide

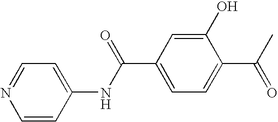

To a solution of the 4-acetyl-3-hydroxy-benzoic acid (prepared by a method known per se, 1.7 g) in dimethylformamide (DMF) (0.25 M), diisopropylethylamine (DIEA) (3 eq.), 1-Hydroxybenzotriazole (HOBt) (0.3 eq.) and TBTU (1.3 eq.) were added. The reaction mixture was stirred at RT for 5 minutes and the 4-aminopyridine (1 eq.) was added. The reaction mixture was stirred at RT for 3 hours and then, was evaporated. The residue was dissolved in EtOAc. The organic layer was washed with 1M NaHCO₃ and extracted with 0.1 N HCl. The acidic aqueous layer was neutralized with 1M NaHCO₃ to pH=8 and extracted with EtOAc. The organic layer was dried over MgSO₄ and evaporated to give a yellow powder (50% yield). LC/MS: 1.34 min, ES⁺: 257; ES⁻: 255.

### Intermediate 2: {1-[2-amino-4-(pyridin-4-ylcarbamoyl)-phenyl]-ethyl}-carbamic acid tert-butyl ester

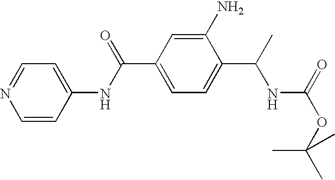

To a solution of 4-acetylbenzoic acid methyl ester (5 g) in EtOH (0.25 M) were successively added DIEA (4 eq.) and hydroxylamine hydrochloride (3 eq.). The reaction mixture was stirred overnight at 55° C. The reaction mixture was cooled down at room temperature (RT) and the solvent was evaporated. Water was added to the residue. The aqueous layer was extracted with dichloromethane (DCM) (3×100 ml). The combined organic layers were washed with brine and dried over MgSO₄. The solvent was evaporated yielding the 4-[1-(hydroxyimino)-ethyl]-benzoic acid methyl ester as a beige powder (91% yield, 5.2 g).

To a solution of 4-[1-(hydroxyimino)-ethyl]-benzoic acid methyl ester (5.2 g) in acetic acid was added activated zinc (10 eq.). The reaction mixture was stirred at RT for 1 hour. Zinc was removed by filtration. The filtrate was evaporated. The residue was diluted with 1N NaOH and extracted with DCM. The combined organic layers were dried over MgSO₄. The solvent was removed under vacuum. The resulting solid was dissolved in 1N HCl and then was lyophilized, yielding the 4-(1-amino-ethyl)-benzoic acid methyl ester hydrochloric acid salt as a beige powder (81% yield).

The 4-(1-amino-ethyl)-benzoic acid methyl ester hydrochloric acid salt (4.7 g) was suspended in DCM and cooled at 0° C. DIEA (3 eq.) and trifluoroacetic anhydride (1.1 eq.) were successively added and the reaction mixture was stirred for 2 h at 0° C. The reaction mixture was diluted with DCM. The organic layer was successively washed with 1M Na₂CO₃, 1M HCl and brine. The organic layer was dried over MgSO₄. DCM was evaporated to give 5.8 g of 4-[1-(2,2,2-trifluoro-acetylamino)-ethyl]-benzoic acid methyl ester as a beige gum (98% yield).

The 4-[1-(2,2,2-trifluoro-acetylamino)-ethyl]-benzoic acid methyl ester (4.6 g) was dissolved in sulfuric acid at −30° C. and concentrated nitric acid (1.1 eq.) was added. The reaction mixture was stirred for 2 hours from −30° C. to −10° C. Water was added at −10° C. and DCM was then added. The organic layer was washed with water and brine, dried over MgSO₄. DCM was evaporated to give the 3-nitro-4-[1-(2,2,2-trifluoro-acetylamino)-ethyl]-benzoic acid methyl ester as an orange powder (100% yield).

To a solution of 3-nitro-4-[1-(2,2,2-trifluoro-acetylamino)-ethyl]-benzoic acid methyl ester (5.4 g) in EtOH was added NaOH (4 eq.). The reaction mixture was stirred at 40° C. for 1 hour. The reaction mixture was neutralized with 1N HCl until pH=7. Water was evaporated yielding 3.5 g of 4-(1-Amino-ethyl)-3-nitro-benzoic acid in mixture with salts. The crude residue was dissolved in a mixture EtOH/water. Na₂CO₃ (2 eq.) and (BOC)₂O (1.1 eq.) were added. The reaction mixture was stirred at RT for 12 hours and then was diluted with DCM. The aqueous layer was extracted with DCM. The combined organic layers were washed with brine and then, dried over MgSO₄. DCM was evaporated to give the 4-(1-tert-butoxycarbonylamino-ethyl)-3-nitro-benzoic acid, which was used without further purification. The crude 4-(1-tert-butoxycarbonylamino-ethyl)-3-nitro-benzoic acid (3.6 g) was dissolved in DMF (0.25 M). DIEA (3 eq.), HOBt (0.3 eq.) and TBTU (1.3 eq.) were added. The reaction mixture was stirred at RT for 5 minutes and the 4-aminopyridine (1 eq.) was added. The reaction mixture was stirred at RT for 30 minutes and then, was evaporated. The residue was dissolved in saturated Na₂CO₃ aqueous solution. The aqueous phase was extracted with DCM (3×100 ml). The combined organic layers were dried over MgSO₄ and then, were evaporated. The {1-[2-nitro-4-(pyridin-4-ylcarbamoyl)-phenyl]-ethyl}-carbamic acid tert-butyl ester was purified by flash chromatography (DCM/cyclohexane/MeOH: 1/1/0; 1/0/0 and 4/0/1), yielding the {1-[2-nitro-4-(pyridin-4-ylcarbamoyl)-phenyl]-ethyl}-carbamic acid tert-butyl ester as a pale brown powder (62% yield).

To a solution of {1-[2-nitro-4-(pyridin-4-ylcarbamoyl)-phenyl]-ethyl}-carbamic acid tert-butyl ester (2.96 g) in MeOH was added Pd/C (10%). The reaction mixture was stirred at RT, under 5 atm of hydrogen for 6 hours. Pd/C was removed by filtration. The filtrate was evaporated. The residue was taken in DCM. The organic layer was washed with water and brine, was then dried over MgSO₄ and was evaporated, yielding the title product as a beige powder (83% yield). Tᵣₑₜ: 1.48 min, ES⁺: 357; ES⁻: 355. Purity: 90%.

### Intermediate 3: 4-acetyl-3-methanesulfonyloxy-benzoic acid ethyl ester

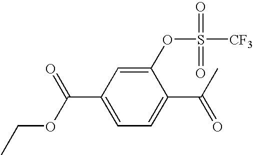

To a solution of 1-(4-bromo-2-hydroxy-phenyl)-ethanone (1 g) in a mixture of 13 ml of THF and 2 ml of water, were added potassium acetate (1 eq.), 1.3-bis-diphenylphosphinopropane (0.02 eq.) and palladium-(II)-acetate (0.04 eq.). The reaction mixture was stirred under 50 atm of CO, at 150° C. for 3 hours and then, filtered. The filtrate was dried over MgSO₄ and evaporated to give 819 mg of 4-acetyl-3-hydroxy-benzoic acid as a white powder, which will be used without further purification (98% yield).

To a solution of 4-acetyl-3-hydroxy-benzoic acid (819 mg) in ethanol (8 ml) was added 2 ml of concentrated HCl. The mixture was stirred for 4 hours at 100° C. and evaporated. The residue was purified by flash chromatography on silica gel (DCM/MeOH: 95/5) to give 600 mg of 4-acetyl-3-hydroxy-benzoic acid ethyl ester as white powder (64% yield).

To a solution of 4-acetyl-3-hydroxy-benzoic acid ethyl ester (550 mg) in DCM (10 ml) was added pyridine (2 eq.). The mixture was cooled to 0° C. and triflic anhydride (1.1 eq.) was added dropwise. The mixture was stirred at 0° C. for 1 hour and at RT for 3 hours. 1.6 g of a crude brown solid was obtained to be directly used to the next step (mixture with salts). Tᵣₑₜ: 2.75 min, ES⁺: 287; ES⁻: 285.

### Intermediate 4: 4-acetyl-3-bromo-N-pyridin-4-yl-benzamide

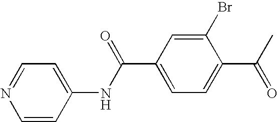

Bromine (7 ml, 0.14 mol) was added to a solution of 4-ethylbenzoic acid (15.0 g, 0.10 mol) in acetic acid (300 ml), nitric acid (65 ml) and water (50 ml). A solution of silver nitrate (17.0 g, 0.10 mmol) in water (50 ml) was added dropwise while vigorously stirring. The reaction mixture was stirred overnight at room temperature at which a yellow solid precipitated. The precipitate was collected by filtration, washed extensively with water and was extracted several times with CHCl₃. The solution was washed with water and the solvent was removed under reduced pressure, yielding the 3-Bromo-4-ethylbenzoic acid (11.8 g, 52% yield) as a white solid. ¹H NMR (300 MHz, CDCl₃): 1.26 ppm (t, 3H, J=7.8 Hz); 2.83 ppm (q, 2H, J=7.8 Hz); 7.34 ppm (d, 1H, J=8.1 Hz); 7.97 ppm (dd, 1H, J=8.1 & 1.8 Hz); 8.27 ppm (d, 1H, J=1.8 Hz).

Chromium(VI) oxide (24.2 g, 24.2 mmol) was dissolved in a mixture of acetic acid (100 ml) and acetic anhydride (70 ml). A solution of 3-Bromo-4-ethylbenzoic acid (10.31 g, 45.2 mmol) in acetic acid (100 ml) was added dropwise. The 3-Bromo-4-ethylbenzoic acid partly crystallized in the dropping funnel and was washed away with acetic acid (2×20 ml). Overnight the reaction mixture was stirred under a nitrogen atmosphere at room temperature. After the addition of water (1 L) the mixture was extracted with diethyl ether (4×200 ml). The collected organic layers were washed with water, added active charcoal and filtrated. The 4-acetyl-3-bromobenzoic acid (8.12 g, 74% yield) was isolated as a greenish solid. ¹H NMR (300 MHz, CDCl₃): 2.66 ppm (s, 3H); 7.51 ppm (d, 1H, J=8.1 Hz); 8.10 ppm (dd, 1H, J=8.1 Hz & 0.1 Hz); 8.35 ppm (d, 1H, J=0.1 Hz).

To a solution of 4-acetyl-3-bromobenzoic acid (6.64 g, 26.6 mmol) in dioxane (130 ml) and DMF (1 ml) was added 4-aminopyridine (5.02 g, 53.1 mmol), DIPEA (5.0 ml, 53.1 mmol) and TBTU (13.13 g, 40.0 mmol). The mixture was stirred overnight under a nitrogen atmosphere at room temperature. After quenching with water the mixture was extracted with TBME (3×). The collected organic layers were washed with water, dried with Na₂SO₄ and filtrated. The solvent was removed under reduced pressure and purified by column chromatography (Al₂O₃, heptane/ethyl acetate gradient). The 4-Acetyl-3-bromo-N-pyridin-4-yl-benzamide (6.65 g, 78%) was isolated as a yellow oil. ¹H NMR (300 MHz, CDCl₃): 2.63 ppm (s, 3H); 7.43 ppm (d, 1H, J=8.0 Hz); 7.67 ppm (d, 2H, J=4.7 & 1.7 Hz); 7.83 ppm (d, 1H, J=8.0 Hz); 8.08 ppm (d, 1H, J=1.7 Hz); 8.51 ppm (dd, 2H, J=4.7 & 1.7 Hz); 9.21 ppm (s, 1H).

### Intermediate 5: 4-iodo-1-triisopropylsilanyl-1H-pyrrolo[2,3-b]pyridine

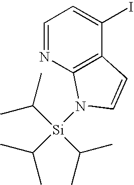

1H-Pyrrolo[2,3-b]pyridine (10 g) was dissolved in DME/heptane ((1:2), 200 ml). The reaction mixture was cooled down to 0° C. and mCPBA (2.1 eq.) was added slowly. The reaction mixture turned yellow and a precipitate was formed. DME-heptane (1:2) mixture (50 ml) was added and the slurry was stirred at RT for 6.5 hours. The precipitate was filtered out and washed with DME-heptane (1:2). Slurry of the salt of azaindole N-oxide in water (100 ml) was treated with 30% (in mass) K₂CO₃ to raise the pH to about 9.5-10.5. First, a dark solution was formed. The slurry was cooled to 0 to 5° C. for 16 hours and then filtered to recover the precipitate. The precipitate was washed with additional water and then dried to provide 1H-pyrrolo[2,3-b]pyridine 7-oxide as a pink powder (70% yield).

The 1H-pyrrolo[2,3-b]pyridine 7-oxide (3.67 g) and tetramethylammonium bromide (1.5 eq.) were placed in DMF (15 ml). The mixture was cooled to 0° C. and methanesulfonic anhydride (2 eq.) was added portion wise. The suspension was allowed to reach RT and stirred for 5.5 hours. The reaction mixture was then poured into water (70 ml) and neutralized with 4M NaOH. Water (60 ml) was added. The product was extracted with DCM, washed with water, dried over MgSO4, filtered. The solvent was evaporated and the residue was purified by flash chromatography on silica gel (DCM to DCM/MeOH 9:1) to give the 4-bromo-1H-pyrrolo[2,3-b]pyridine as a yellow oil (21% yield).

A flask was charged with 4-bromo-1H-pyrrolo[2,3-b]pyridine (1.5 g) and dry THF (12 ml). The mixture was cooled to 0° C. and sodium hydride (418 mg, 60% dispersion in mineral oil) was added portion wise. After 15 min, chloro-triisopropylsilane (0.75 eq.) was added and the flask was sealed and heated at 80° C. for 3.5 hours. The reaction mixture was cooled down to RT, neutralized with saturated NH₄Cl solution and extracted with hexanes. Combined organic layers were dried over MgSO₄ and concentrated under vacuum. Filtration through a small column with silica gel (eluent: 100% hexanes) gave the 4-bromo-1-triisopropylsilanyl-1H-pyrrolo[2,3-b]pyridine as a colorless oil (15% yield).

A two-necked round-bottom flask was dried in the flow of nitrogen. The flask was charged with 4-bromo-1-triisopropylsilanyl-1H-pyrrolo[2,3-b]pyridine (140 mg) and dry THF (3 ml). The mixture was cooled to −85° C. and a solution of tert-butyl lithium (1.5M in pentane, 1.6 eq.) was added dropwise. After 15 min (yellow color), iodine (1 eq.) in THF (2 ml) was added. After 50 min, a saturated aqueous solution of ammonium chloride was added and the mixture was allowed to reach RT. The product was extracted with ethyl acetate (×3), washed with Na₂S₂O₃ solution and water, and then dried over MgSO₄. The solvent was evaporated to provide a mixture of the iodide and de-brominated compound (4:1). The 4-iodo-1-triisopropylsilanyl-1H-pyrrolo[2,3-b]pyridine was purified by flash chromatography on silica gel (hexane 100%) to give the title compound as a colorless oil (46% yield).

### Example 2

### General Synthesis Procedures

### Protocol A:

To a solution of Intermediate 3 (1 eq.) in a mixture of toluene and MeOH (4/1) were added sodium carbonate (4 eq.), the corresponding boronic acid (1.5 eq.) and tetrakis(triphenylphosphine) palladium(0) (0.2 eq.). The mixture was stirred at 75° C. for 1 hour and toluene was added. The organic layer was washed with 1M Na₂CO₃, dried over MgSO₄ and evaporated. The residue was purified by semi-prep LC/MS.

To a solution of the previous compound (1 eq.) in 1,4-dioxane (0.25 M) was added 1M NaOH (1 eq.). The mixture was stirred for 15 min at 50° C. 1N HCl (until pH=2) and DCM were added to the mixture. The compound was extracted with DCM. The organic layer was dried over MgSO₄ and evaporated.

The previous benzoic acid derivative was coupled with 4-aminopyridine, according to the protocol described for Intermediate 1 or Intermediate 2.

The previous ketone derivative was converted to the oxime according to the procedure described for Intermediate 2. The oxime was finally reduced according to the protocol described for Intermediate 2.

The final compound was purified either by flash chromatography on silica gel or preparative HPLC.

### Protocol B:

To a solution of Intermediate 4 (1 eq.) in toluene (0.25 M, with a few drops of MeOH if necessary) were successively added sodium bicarbonate (4 eq.), the corresponding boronic acid (1.5 eq.) and tetrakis(triphenylphosphine) palladium(0) (0.2 eq.). The reaction mixture was stirred at 70° C. for 12 to 48 hours and then, was cooled down at RT. The solvent was evaporated. The residue was partitioned between DCM and 0.5N HCl. The product was extracted with 0.5N HCl and then, the pH of the aqueous layer was brought to 12 (with 2N NaOH). The compound was extracted with EtOAc. The organic layer was dried over MgSO₄ and evaporated. The residue was purified by preparative HPLC or was used without further purification for the next step.

The previous ketone derivative was converted to the oxime according to the procedure described for Intermediate 2. The oxime was finally reduced according to the protocol described for Intermediate 2.

The final compound was purified either by flash chromatography on silica gel or preparative HPLC.

### Protocol C:

To a solution of Intermediate 1 (1 eq.) in DMF (0.25 M) were added K₂CO₃ (10 eq.) and the corresponding halogenated derivative (5 eq.): 2-bromoethanol, 3-bromopropanol, (2-Bromo-ethyl)-carbamic acid tert-butyl ester, (3-Bromo-propyl)-carbamic acid tert-butyl ester and benzyl bromide, respectively for the preparation of the Compounds 8, 9, 10, 11 and 12. The reaction mixture was stirred at 60° C. overnight and then, was evaporated. The residue was partitioned between water and EtOAc. The product was extracted with EtOAc. The combined organic layers were dried over MgSO₄ and evaporated. The residue was purified either by flash chromatography on silica gel or preparative HPLC.

To a solution of the previous compound (1 eq.) in absolute ethanol (0.25 M), were added DIEA (1.6 eq.) and hydroxylamine hydrochloride (1.6 eq.). The reaction mixture was stirred at 80° C. for 2 to 12 hours and then, cooled down at RT. The solvent was evaporated (in the case the intermediate oxime contained a free alcohol, the next step was launched without further work up). The residue was taken in water. The solid was collected by filtration and washed with water.

The oxime (1 eq.) was dissolved in acetic acid (0.25 M) and activated zinc (10 eq.) was added. The reaction mixture was stirred at RT for 3 hours. Zinc was filtered off and the filtrate was evaporated. The residue was taken in 2N NaOH (pH=14). The product was extracted with EtOAc. The organic layer was washed with brine, dried over MgSO₄ and finally was evaporated.

When appropriate, the tert-butyloxycarbonyl group was removed using HCl gas (or 3N HCl) in a suitable solvent (methanol, diethyl ether, or 1,4-dioxane).

### Protocol D:

To a solution of Intermediate 2 (1 eq.) in DMF (0.25 M) was added the corresponding isocyanate or isothiocyanate (1.2 eq.). The reaction mixture was stirred at RT for 2 to 4 hours. DMF was evaporated and water was added to the residue. The product was extracted with EtOAc or DCM. The combined organic layers were dried over MgSO₄ and then, were evaporated. The residue was purified either by flash chromatography on silica gel or preparative HPLC.

The tert-butyloxycarbonyl group was removed using HCl gas (or 3N HCl) in a suitable solvent (methanol, diethyl ether, or 1,4-dioxane).

### Protocol E:

To a suspension (or solution) of the Intermediate 2 (1 eq.) in toluene (0.25 M) were successively added sodium carbonate (4 eq.), the corresponding halogenated derivative (1.5 eq.) and the tetrakis(triphenylphosphine) palladium(0) (0.2 eq.). The reaction mixture was stirred at 60° C. overnight. The toluene was evaporated and DCM was added to the residue. The organic layer was washed with water and brine, then dried over MgSO₄. The product was extracted with 1N HCl. Water was evaporated. The residue was purified by flash chromatography on silica gel or preparative HPLC, or was used without further purification.

The tert-butyloxycarbonyl group was removed using HCl gas (or 3N HCl) in a suitable solvent (methanol, diethyl ether, or 1,4-dioxane).

### Protocol F:

To a solution of the Intermediate 2 (1 eq.) in DMF (0.25 M) was added the corresponding aldehyde (1.5 eq.). The reaction mixture was stirred at RT for 20 min. Then, NaHB(OAc)₃ (5 eq.) was added by portion. The reaction mixture was stirred at RT for 6 hours. Water was added to the mixture and the product was extracted with DCM. The combined organic layers were dried over MgSO₄ and then, partially evaporated. NaBH₄ (5 eq.) was added and the mixture was stirred overnight. 1N HCl was added to the mixture to pH=7 and DCM was added. The organic layer was washed with water and brine, dried over MgSO₄ and evaporated. The residue was purified either by flash chromatography on silica gel or preparative HPLC.

The tert-butyloxycarbonyl group was removed using HCl gas (or 3N HCl) in a suitable solvent (methanol, diethyl ether, or 1,4-dioxane).

### Protocol G:

To a suspension (or solution) of the corresponding carboxylic acid (1 eq.) in DCM (0.25 M), were added oxalyl chloride (2.5 eq.) and DMF (1 drop). The mixture was stirred at RT for 2 hours, and then evaporated, yielding the corresponding acyl chloride, which was used without further purification.

The acyl chloride was dissolved in a minimum of acetonitrile, and then added to a solution of Intermediate 2 (1 eq.) in acetonitrile (0.25 M). The reaction mixture was stirred for 2 to 12 hours at RT (or at 40° C.) under a nitrogen atmosphere. The reaction mixture was evaporated. The residue was partitioned between water and DCM. The organic layer was washed with brine, and then was dried over MgSO₄. The organic layer was evaporated. The residue was purified either by flash chromatography on silica gel or preparative HPLC.

The tert-butyloxycarbonyl group was removed using HCl gas (or 3N HCl) in a suitable solvent (methanol, diethyl ether, or 1,4-dioxane).

### Example 3

### Synthesis of Compounds of the Invention

### Attribution of the Configuration:

The Cahn-Ingold-Prelog system was used to attribute the absolute configuration of chiral center, in which the four groups on an asymmetric carbon are ranked to a set of sequences rules. Reference is made to Cahn; Ingold; Prelog Angew. Chem. Int. Ed. Engl. 1966, 5, 385-415.

### Name of the Molecule

The software MDL ISIS™/Draw 2.5 was used to assign the name of the molecules.

### Compound 1: 4-(1-amino-ethyl)-3-hydroxy-N-pyridin-4-yl-benzamide dihydrochloric acid salt

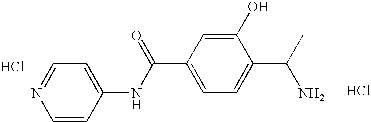

To a solution of Intermediate 1 (104 mg) in absolute ethanol, were added DIEA (1.6 eq.) and hydroxylamine hydrochloride (1.6 eq.). The reaction mixture was stirred at 60° C. for 2 hours and then, cooled down at RT. The solvent was evaporated. The residue was taken in water. The solid was collected by filtration and washed with water. The 3-hydroxy-4-[1-(hydroxyimino)-ethyl]-N-pyridin-4-yl-benzamide was obtained as a white powder (66% yield).

The 3-hydroxy-4-[1-(hydroxyimino)-ethyl]-N-pyridin-4-yl-benzamide (73 mg) was dissolved in acetic acid (1.5 ml) and activated zinc (10 eq.) was added. The reaction mixture was stirred at RT for 3 hours. Zinc was filtered off and the filtrate was evaporated. The residue was dissolved in water. The product was purified by column on ion exchange resin (DOWEX 50WX4-400). After formation of the hydrochloric acid salt, the title compound was obtained as a beige powder (77% yield). Tᵣₑₜ: 0.30 min, ES⁺: 258; ES⁻: 256. Purity: 96%. ¹H NMR (300 MHz, DMSO-d6): 1.54 ppm (d, 3H, J=6.9 Hz); 4.63 ppm (m, 1H); 7.31 ppm (s, 1H); 7.36 ppm (s, 2H); 8.10 ppm (d, 2H, J=7.5 Hz); 8.47 ppm (d, 2H, J=7.5 Hz).

### Compound 2: 4-(1-amino-ethyl)-3-nitro-N-pyridin-4-yl-benzamide dihydrochloric acid salt

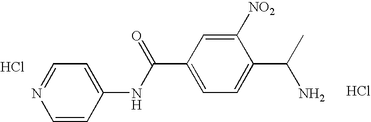

The {1-[2-nitro-4-(pyridin-4-ylcarbamoyl)-phenyl]-ethyl}-carbamic acid tert-butyl ester (preparation described for Intermediate 2, 71 mg) was dissolved in 3N HCl. The reaction mixture was stirred at RT for 2 hours. After evaporation of the water, the residue was purified by preparative HPLC, yielding the title compound as a white powder (46% yield). Tᵣₑₜ: 0.81 min, ES⁺:288; ES⁻: 286.

Purity: 100%. ¹H NMR (300 MHz, DMSO-d6): 1.59 ppm (d, 3H, J=6.6 Hz); 4.90 ppm (m, 1H); 8.01 ppm (d, 1H, J=8.3 Hz); 8.21 ppm (d, 2H, J=7.2 Hz); 8.39 ppm (dd, 1H, J=8.3 & 1.8 Hz); 8.62 ppm (d, 1H, J=1.8 Hz); 8.68 ppm (d, 2H, J=7.2 Hz).

### Compound 3: 3-amino-4-(1-amino-ethyl)-N-pyridin-4-yl-benzamide dihydrochloric acid salt

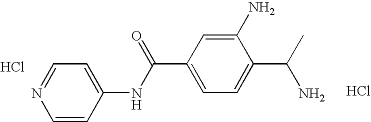

The title compound was prepared according to the procedure of Compound 2, starting from Intermediate 2. The compound was purified by preparative HPLC, yielding the title compound as a white powder (82% yield). Tᵣₑₜ: 0.69 min, ES⁺:258; ES⁻: 256. Purity: 100%. ¹H NMR (300 MHz, DMSO-d6): 1.44 ppm (d, 3H, J=6.6 Hz); 4.57 ppm (m, 1H); 7.33 ppm (d, 1H, J=1.7 Hz); 7.39 ppm (d, 1H, J=8.3 Hz); 7.49 ppm (d, 1H, J=8.3 Hz); 8.34 ppm (d, 2H, J=7.2 Hz); 8.74 ppm (d, 2H, J=7.2 Hz).

### Compound 4: 6-(1-amino-ethyl)-4′-chloro-biphenyl-3-carboxylic acid pyridin-4-ylamide dihydrochloric acid salt

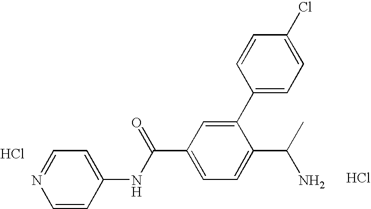

The title compound was prepared according to the general Protocol A or B, starting from Intermediate 3 and 4-chlorophenyl-boronic acid, yielding a beige powder (8% overall yield using procedure A, and 20% using procedure B). Tᵣₑₜ: 1.30 min, ES⁺:353; ES⁻: 351. Purity: 100%. ¹H NMR (300 MHz, DMSO-d6): 1.45 ppm (d, 3H, J=6.9 Hz); 4.27 ppm (m, 1H); 7.49 ppm (d, 2H, J=8.4 Hz); 7.60 ppm (d, 2H, J=8.4 Hz); 7.94 ppm (d, 1H, J=1.5 Hz); 8.03 ppm (d, 1H, J=8.4 Hz); 8.20 ppm (dd, 1H, J=8.4 & 1.5 Hz); 8.30 ppm (d, 2H, J=6.4 Hz); 8.72 ppm (d, 2H, J=6.4 Hz); 11.70 ppm (s, 1H).

### Compound 5: 4-(1-amino-ethyl)-N-pyridin-4-yl-3-thiophen-2-yl-benzamide dihydrochloric acid salt

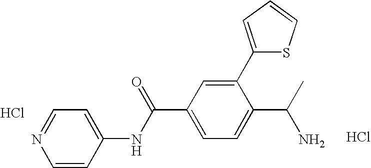

The title compound was prepared according to the general Protocol A, starting from Intermediate 3 and 2-thiophene boronic acid, yielding a beige powder (5.6% overall yield). Tᵣₑₜ: 1.09 min, ES⁺:324; ES⁻: 322. Purity: 100%. ¹H NMR (300 MHz, DMSO-d6): 1.52 ppm (d, 3H, J=6.9 Hz); 4.53 ppm (m, 1H); 7.23 ppm (m, 1H); 7.30 ppm (dd, 1H, J=5.1 & 1.2 Hz); 7.75 ppm (dd, 1H, J=5.1 & 1.2 Hz); 8.06 ppm (d, 1H, J=1.5 Hz); 8.09 ppm (d, 1H, J=8.1 Hz); 8.25 ppm (dd, 1H, J=8.1 & 1.5 Hz); 8.38 ppm (d, 2H, J=7.2 Hz); 8.74 ppm (d, 2H, J=7.2 Hz); 11.84 ppm (s, 1H).

### Compound 6: 6-(1-amino-ethyl)-4′-methoxy-biphenyl-3-carboxylic acid pyridin-4-ylamide dihydrochloric acid salt

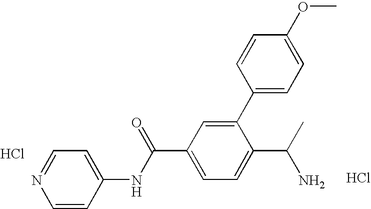

The title compound was prepared according to the general Protocol A, starting from Intermediate 3 and 4-methoxyphenyl-boronic acid, yielding a white powder (5.6% overall yield). Tᵣₑₜ: 1.25 min, ES⁺:348; ES⁻: 346. Purity: 97%. ¹H NMR (300 MHz, DMSO-d6): 1.50 ppm (d, 3H, J=6.6 Hz); 4.55 ppm (m, 1H); 7.08 ppm (d, 2H, J=8.7 Hz); 7.37 ppm (d, 2H, J=8.7 Hz); 7.92 ppm (d, 1H, J=1.5 Hz); 8.00 ppm (d, 1H, J=8.4 Hz); 8.16 ppm (dd, 1H, J=8.4 & 1.5 Hz); 8.33 ppm (d, 2H, J=6.9 Hz); 8.73 ppm (d, 2H, J=6.9 Hz); 11.69 ppm (s, 1H).

### Compound 7: 4-(1-amino-ethyl)-3-furan-3-yl-N-pyridin-4-yl-benzamide dihydrochloric acid salt

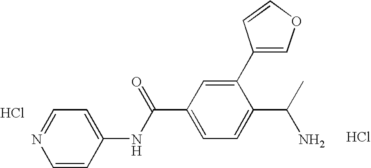

The title compound was prepared according to the general Protocol A, starting from Intermediate 3 and 3-furanboronic acid, yielding a beige powder (8.4% overall yield). Tᵣₑₜ: 0.85 min, ES⁺:308; ES⁻: 306. Purity: 97%. ¹H NMR (300 MHz, DMSO-d6): 1.50 ppm (d, 3H, J=6.6 Hz); 4.55 ppm (m, 1H); 6.86 ppm (s, 1H); 7.87 ppm (t, 1H, J=1.8 Hz); 8.00-8.03 ppm (m, 2H); 8.06 ppm (d, 1H, J=1.5 Hz); 8.15 ppm (dd, 1H, J=8.1 & 1.5 Hz); 8.42 ppm (d, 2H, J=7.2 Hz); 8.74 ppm (d, 2H, J=7.2 Hz); 11.89 ppm (s, 1H).

### Compound 8: 4-(1-amino-ethyl)-3-(2-hydroxy-ethoxy)-N-pyridin-4-yl-benzamide dihydrochloric acid salt

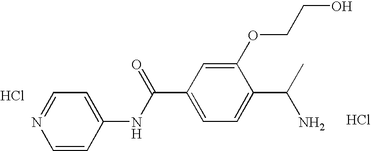

The title compound was prepared according to the general Protocol C, starting from Intermediate 1 and 2-bromoethanol, yielding a white powder (29% overall yield). Tᵣₑₜ: 1.00 min, ES⁺:302; ES⁻: 300. Purity: 100%. NMR (300 MHz, DMSO-d6): 1.50 ppm (d, 3H, J=6.9 Hz); 3.77 ppm (t, 2H, J=4.8 Hz); 4.22 ppm (t, 2H, J=4.8 Hz,); 4.70 ppm (m, 1H); 7.63 ppm (d, 1H, J=8.1 Hz); 7.75 ppm (dd, 1H, J=8.1 & 1.5 Hz,); 7.78 (d, 1H, J=1.5 Hz); 8.45 ppm (d, 2H, J=7.2 Hz); 8.74 ppm (d, 2H, J=7.2 Hz); 11.91 (s, 1H).

### Compound 9: 4-(1-amino-ethyl)-3-(3-hydroxy-propoxy)-N-pyridin-4-yl-benzamide dihydrochloric acid salt

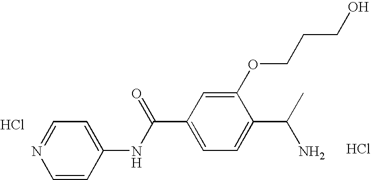

The title compound was prepared according to the general Protocol C, starting from Intermediate 1 and 3-bromopropanol, yielding a white powder (10% overall yield). Tᵣₑₜ: 1.06 min, ES⁺:316; ES⁻: 314. Purity: 95%. NMR (300 MHz, DMSO-d6+D₂O): 1.47 ppm (d, 3H, J=6.9 Hz); 1.94 ppm (m, 2H); 3.59 ppm (t, 2H, J=6.0 Hz); 4.22 ppm (t, 2H, J=6.0 Hz); 4.66 ppm (m, 1H); 7.58 ppm (d, 1H, J=8.1 Hz); 7.64 ppm (d, 1H, J=1.5 Hz); 7.69 ppm (dd, 1H, J=8.1 & 1.5 Hz); 8.33 ppm (d, 2H, J=7.2 Hz); 8.73 ppm (d, 2H, J=7.2 Hz).

### Compound 10: 3-(2-amino-ethoxy)-4-(1-amino-ethyl)-N-pyridin-4-yl-benzamide trihydrochloric acid salt

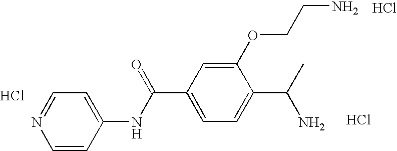

The title compound was prepared according to the general Protocol C, starting from Intermediate 1 and (2-Bromo-ethyl)-carbamic acid tert-butyl ester, yielding a white powder (21% overall yield). Tᵣₑₜ: 0.84 min, ES⁺:301; ES⁻: 299. Purity: 96%. ¹H NMR (300 MHz, D2O): 1.55 ppm (d, 3H, J=6.9 Hz); 3.44 ppm (t, 2H, J=4.8 Hz); 4.37 ppm (t, 2H, J=4.8 Hz); 4.87 ppm (m, 1H); 7.49 ppm (d, 1H, J=7.5 Hz,); 7.51 ppm (s, 1H); 7.57 ppm (d, 1H, J=7.5 Hz); 8.15 ppm (d, 2H, J=7.2 Hz); 8.51 ppm (d, 2H, J=7.2 Hz).

### Compound 11: 4-(1-amino-ethyl)-3-(3-amino-propoxy)-N-pyridin-4-yl-benzamide trihydrochloric acid salt

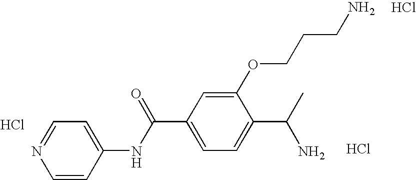

The title compound was prepared according to the general Protocol C, starting from Intermediate 1 and (3-Bromo-propyl)-carbamic acid tert-butyl ester, yielding a white powder (26% overall yield). Tᵣₑₜ: 0.84 min, ES⁺:315; ES⁻: 316. Purity: 96%. ¹H NMR (300 MHz, D2O): 1.55 ppm (d, 3H, J=6.9 Hz); 2.15 ppm (m, 1H); 3.13 ppm (t, 2H, J=7.2 Hz); 4.21 ppm (m, 2H); 4.77 ppm (m, 1H); 7.45 ppm (d, 1H, J=7.8 Hz); 7.48 ppm (d, 1H, J=1.5 Hz); 7.53 ppm (dd, 1H, J=7.8 Hz & 1.5 Hz); 8.15 ppm (d, 2H, J=7.5 Hz); 8.50 ppm (d, 2H, J=7.5 Hz).

### Compound 12: 4-(1-amino-ethyl)-3-benzyloxy-N-pyridin-4-yl-benzamide dihydrochloric acid salt

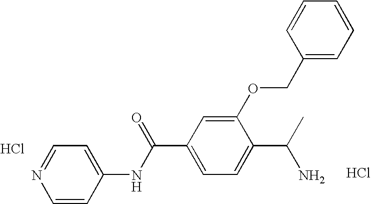

The title compound was prepared according to the general Protocol C, starting from Intermediate 1 and benzyl bromide, yielding a white powder (20% overall yield). Tᵣₑₜ: 1.40 min, ES⁺:348; ES⁻: 346. Purity: 92%. ¹H NMR (300 MHz, D2O): 1.54 ppm (d, 3H, J=6.9 Hz); 4.68 ppm (m, 1H); 5.21 ppm (s, 2H); 7.29-7.52 ppm (m, 8H); 8.11 ppm (d, 2H, J=7.5 Hz); 8.48 ppm (d, 2H, J=7.5 Hz).

### Compound 13: 4-(1-amino-ethyl)-3-(3-phenyl-thioureido)-N-pyridin-4-yl-benzamide dihydrochloric acid salt

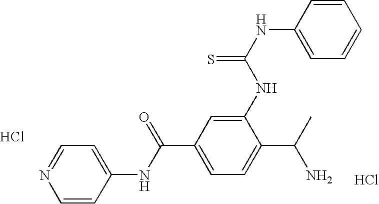

The title compound was prepared according to the general Protocol D, starting from Intermediate 2 and phenylisothiocyanate, yielding a beige powder (35% overall yield). Tᵣₑₜ: 0.98 min, ES⁺:392; ES⁻: 390. Purity: 95%. ¹H NMR (300 MHz, DMSO-d6): 1.54 ppm (d, 3H, J=6.9 Hz); 4.62 ppm (m, 1H); 7.15 ppm (t, 1H, J=7.4 Hz); 7.35 ppm (dd, 1H, J=7.4 Hz); 7.59 ppm (d, 2H, J=7.4 Hz); 7.83 ppm (d, 1H, J=8.2 Hz); 8.03 ppm (s, 1H); 8.33 ppm (d, 2H, J=7.1 Hz); 8.75 ppm (d, 2H, J=7.1 Hz); 10.03 ppm (s, 1H); 10.62 ppm (s, 1H); 11.62 ppm (s, 1H).

### Compound 14: 4-(1-amino-ethyl)-3-(3-phenyl-ureido)-N-pyridin-4-yl-benzamide dihydrochloric acid salt

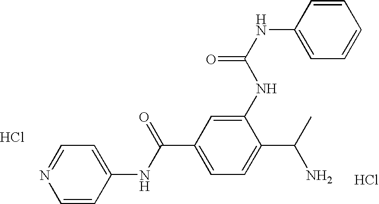

The title compound was prepared according to the general Protocol D, starting from Intermediate 2 and phenylisocyanate, yielding a beige powder (82% overall yield). Tᵣₑₜ: 1.05 min, ES⁺:376; ES⁻: 374. Purity: 100%. ¹H NMR (300 MHz, DMSO-d6): 1.53 ppm (d, 3H, J=6.9 Hz); 4.90 ppm (m, 1H); 7.15 ppm (t, 1H, J=7.4 Hz); 7.27 ppm (dd, 1H, J=7.5 Hz); 7.48 ppm (d, 2H, J=7.5 Hz); 7.78 ppm (d, 1H, J=8.2 Hz); 7.89 ppm (dd, 1H, J=8.2 & 1.6 Hz,); 8.32 ppm (d, 2H, J=7.5 Hz); 8.38 ppm (d, 1H, J=1.8 Hz); 8.75 ppm (d, 2H, J=7.5 Hz); 9.22 ppm (s, 1H); 9.86 ppm (s, 1H); 11.61 ppm (s, 1H).

### Compound 15: 4-(1-amino-ethyl)-3-benzylamino-N-pyridin-4-yl-benzamide dihydrochloric acid salt

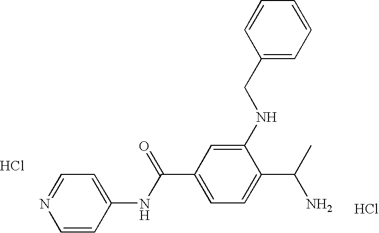

The title compound was prepared according to the general Protocol E, starting from Intermediate 2 and benzyl bromide, yielding a yellow powder (72% overall yield). Tᵣₑₜ: 1.08 min, ES⁺: 347. Purity: 100%. ¹H NMR (300 MHz, DMSO-d6): 1.46 ppm (d, 3H, J=6.6 Hz); 3.90-4.60 ppm (bs, 1H); 4.55-4.65 ppm (m, 1H); 5.70 ppm (s, 2H); 7.35-7.50 ppm (m, 6H); 7.59 ppm (m, 2H); 8.44 ppm (d, 2H, J=6.9 Hz); 8.60-8.75 ppm (m, 2H); 8.97 ppm (d, 2H, J=7.0 Hz); 11.87 ppm (s, 1H).

### Compound 16: 4-(1-amino-ethyl)-3-[(furan-2-ylmethyl)-amino]-N-pyridin-4-yl-benzamide dihydrochloric acid salt

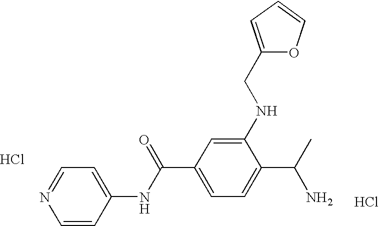

The title compound was prepared according to the general Protocol F, starting from Intermediate 2 and 2-furaldehyde, yielding a pale brown powder (18% overall yield). Tᵣₑₜ: 1.01 min, ES⁺: 337; ES⁻: 335. Purity: 98%. ¹H NMR (300 MHz, DMSO-d6): 1.44 ppm (d, 3H, J=6.5 Hz); 4.45 ppm (bs, 2H); 4.65-4.70 ppm (m, 1H); 6.31 ppm (d, 1H, J=3.2 Hz); 6.36 ppm (dd, 1H, J=3.2 & 1.8 Hz); 7.36 ppm (d, 1H, J=1.6 Hz); 7.44 ppm (dd, 1H, J=8.0 & 1.5 Hz); 7.51-7.56 ppm (m, 2H); 8.44 ppm (d, 2H, J=7.3 Hz); 8.60-8.65 ppm (m, 3H); 8.73 ppm (d, 2H, J=7.3 Hz); 11.75 ppm (s, 1H).

### Compound 17: 4-(1-amino-ethyl)-3-benzoylamino-N-pyridin-4-yl-benzamide dihydrochloric acid salt

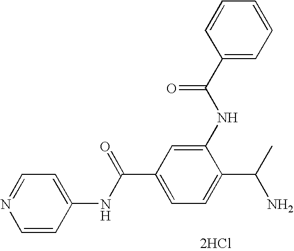

The title compound was prepared according to the general Protocol G, starting from Intermediate 2 and benzoic acid, yielding a pale orange powder (45% overall yield). Tᵣₑₜ: 0.92 min, ES⁺:361; ES⁻: 359. Purity: 95%. ¹H NMR (300 MHz, DMSO-d6): 1.52 ppm (d, 3H, J=6.8 Hz); 4.55-4.63 ppm (m, 1H); 7.52-7.65 ppm (m, 3H); 7.91 ppm (d, 1H, J=8.3 Hz); 8.03-8.10 ppm (m, 3H); 8.14 ppm (d, 1H, J=8.3 Hz); 8.33 ppm (d, 2H, J=6.9 Hz); 8.54-8.63 ppm (m, 2H); 8.74 ppm (d, 2H, J=7.1 Hz); 10.57 ppm (s, 1H); 11.68 ppm (s, 1H).

### Compound 18: furan-2-carboxylic acid [2-(1-amino-ethyl)-5-(pyridin-4-ylcarbamoyl)-phenyl]-amide dihydrochloric acid salt

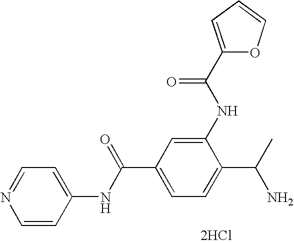

The title compound was prepared according to the general Protocol G, starting from Intermediate 2 and furoic acid, yielding a pale orange powder (22% overall yield). Tᵣₑₜ: 0.84 min, ES⁺:351, ES⁻: 349. Purity: 90%. ¹H NMR (300 MHz, DMSO-d6): 1.52 ppm (d, 3H, J=6.8 Hz); 4.55-4.62 ppm (m, 1H); 6.72 ppm (dd, 1H, J=3.5 & 1.7 Hz); 7.51 ppm (dd, 1H, J=3.5 & 0.6 Hz); 7.93 ppm (d, 1H, J=8.3 Hz); 7.97 ppm (dd, 1H, J=1.3 & 0.6 Hz); 8.02 ppm (d, 1H, J=1.8 Hz); 8.17 ppm (dd, 1H, J=8.3 & 1.8 Hz); 8.39 ppm (d, 2H, J=7.3 Hz); 8.75 ppm (d, 2H, J=7.2 Hz); 10.55 ppm (s, 1H); 11.81 ppm (s, 1H).

### Compound 19: 4-(1-amino-ethyl)-3-(3-methoxy-benzoylamino)-N-pyridin-4-yl-benzamide dihydrochloric acid salt

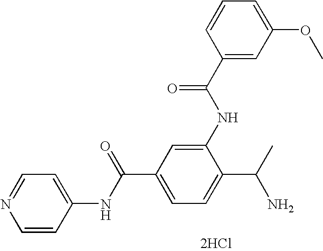

The title compound was prepared according to the general Protocol G, starting from Intermediate 2 and 3-methoxybenzoic acid, yielding a pale orange powder (16% overall yield).

Tᵣₑₜ: 1.05 min, ES⁺: 391, ES⁻: 389. Purity: 99%. ¹H NMR (300 MHz, DMSO-d6): 1.52 ppm (d, 3H, J=6.7 Hz); 3.85 ppm (s, 3H); 4.60 ppm (m, 1H); 7.19 ppm (dd, 1H, J=8.1 & 2.1 Hz); 7.46 ppm (t, 1H, J=8.1 Hz); 7.67 ppm (m, 2H); 7.93 ppm (d, 1H, J=8.3 Hz); 8.05 ppm (d, 1H, J=1.7 Hz); 8.17 ppm (d, 1H, J=8.3 Hz); 8.38 ppm (d, 2H, J=6.9 Hz); 8.67 ppm (m, 2H); 8.75 ppm (d, 2H, J=6.9 Hz); 10.65 ppm (s, 1H); 11.81 ppm (s, 1H).

### Compound 20: 441-amino-ethyl)-3-(3-chloro-benzoylamino)-N-pyridin-4-yl-benzamide dihydrochloric acid salt

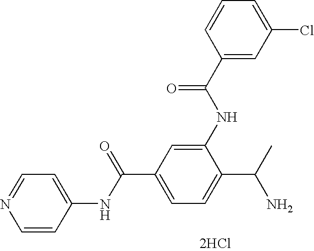

The title compound was prepared according to the general Protocol G, starting from Intermediate 2 and 3-chlorobenzoic acid, yielding a beige powder (44% overall yield). Tᵣₑₜ: 1.13 min, ES⁺: 395; ES⁻: 393. Purity: 99%. ¹H NMR (300 MHz, DMSO-d6): 1.65 ppm (d, 3H, J=6.8 Hz); 4.71-4.78 ppm (m, 1H); 7.72 ppm (t, 1H, J=7.8 Hz); 7.84 ppm (m, 2H); 8.06 ppm (d, 1H, J=8.3 Hz); 8.17 ppm (d, 1H, J=8.4 Hz); 8.27 ppm (m, 1H); 8.30 ppm (d, 2H, J=8.4 Hz); 8.51 ppm (d, 2H, J=7.0 Hz); 8.73-8.80 ppm (m, 2H); 8.89 ppm (d, 2H, J=7.0 Hz); 10.87 ppm (s, 1H); 11.93 ppm (s, 1H).

### Compound 21: 4-(1-Amino-ethyl)-3-(4-methoxy-benzoylamino)-N-pyridin-4-yl-benzamide dihydrochloric acid salt

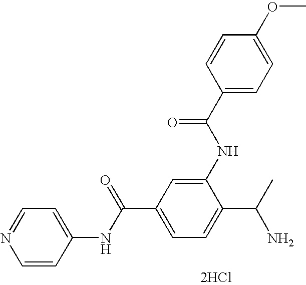

The title compound was prepared according to the general Protocol G, starting from Intermediate 2 and 4-methoxybenzoic acid, yielding a pale green powder (32% overall yield). Tᵣₑₜ: 1.03 min, ES⁺:391, ES⁻: 389. Purity: 99%. ¹H NMR (300 MHz, DMSO-d6): 1.51 ppm (d, 3H, J=6.8 Hz); 3.84 ppm (s, 3H); 4.55-4.60 ppm (m, 1H); 7.09 ppm (d, 2H, J=8.9 Hz); 7.90 ppm (d, 1H, J=8.3 Hz); 8.03 ppm (s, 1H); 8.07 ppm (d, 2H, J=8.8 Hz); 8.14 ppm (d, 1H, J=8.2 Hz); 8.37 ppm (d, 2H, J=6.2 Hz); 8.55-8.65 ppm (m, 2H); 8.76 ppm (d, 2H, J=7.01 Hz); 10.44 ppm (s, 1H); 11.75 ppm (s, 1H).

### Compound 22: 4-(1-Amino-ethyl)-3-(4-chloro-benzoylamino)-N-pyridin-4-yl-benzamide dihydrochloric acid salt

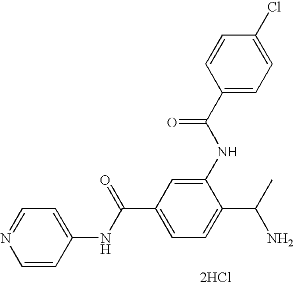

The title compound was prepared according to the general Protocol G, starting from Intermediate 2 and 4-chlorobenzoic acid, yielding a beige powder (10% overall yield). Tᵣₑₜ: 1.16 min, ES⁺:395, ES⁻: 393. Purity: 98%. ¹H NMR (300 MHz, DMSO-d6): 1.51 ppm (d, 3H, J=6.7 Hz); 4.61 ppm (m, 1H); 7.63 ppm (d, 2H, J=8.5 Hz); 7.93 ppm (d, 1H, J=8.3 Hz); 8.05 ppm (s, 1H); 8.12 ppm (d, 2H, J=8.5 Hz); 8.16 ppm (d, 1H, J=8.3 Hz); 8.36 ppm (d, 2H, J=6.8 Hz); 8.65 ppm (m, 2H); 8.75 ppm (d, 2H, J=6.8 Hz); 10.72 ppm (s, 1H); 11.77 ppm (s, 1H).

### Example 4

### ROCK Inhibition

The compounds of the invention were tested for inhibition of human ROCKI/ROCKII mix.

The inhibition assays were performed with a fluorescence polarization (FP) assay using the commercially available ROCK IMAP Kit from Molecular Devices (Product ID. No. R8093) essentially in accordance with the protocol supplied by the manufacturer. The S6 ribosomal protein-derived substrate used was (Fl)-AKRRRLSSLRA, also obtained from Molecular Devices (Product ID No. R7184). The enzyme mix ROCKI/ROCKII was obtained from Upstate Biotechnology (Product ID No 14451).

In summary, all compounds were screened in the wells of a 384 well plate for enzymatic inhibition with concentrations varying from 100 μM to 0.3 nM using a stepwise three (or two)-fold dilution. Y compound (Y-27632 commercially available from Tocris) was used as a reference.

To perform the assay, 1 μl of a solution of the compound to be tested in DMSO (at each concentration) was added to 2 μl of a solution of the enzyme in 10 mM Tris-HCl, 10 mM MgCl₂, 0.1% BSA, 0.05% NaN₃, pH 7,2. The final concentration of the enzyme was 2.6 nM.

After incubating for 30 minutes at room temperature, 2 μl of a mixture of ATP and the protein substrate in 10 mM Tris-HCl, 10 mM MgCl₂, 0.1% BSA, 0.05% NaN₃, pH 7.2 was added. The final concentration of the ATP was 10 μM and final concentration of protein substrate was 0.2 μM.

After incubating for 60 minutes at room temperature, 12 μl of the IMAP Binding Solution (mix of the IMAP Binding Buffer A (1×) and the IMAP Binding Reagent (from the ROCK IMAP kit)) was added.

The mixture thus obtained (total volume: 17 μl) was incubated for 60 minutes at room temperature, upon which the fluorescence polarization was measured using an automated plate reader (Perkin Elmer, Model Envision 2100-0010 HTS) with FP filters: excitation filter FITC FP 480 and emission filters FITC FP P-pol 535 and FITC FP S-pol 535 (Perkin-Elmer). The results were fitted to a curve using the XL-Fit algorithm and IC50 values were calculated for each fitted curve, again using the XL-Fit algorithm.

The IC₅₀ value for the reference compound (Y-27632) was 0.4 μM.

### Example 5

### Compounds of the Invention

In the tables which are set forth below, exemplary compounds of the invention are set out in tabulated form. In these tables, the name of the compound, an arbitrarily assigned compound number and structural information are set out. In addition, the IC₅₀ value obtained (in accordance with the protocol set forth above) is given. The IC₅₀ value obtained (in accordance with the protocol set forth above) is represented as follows: “++++” means IC₅₀ below 0.05 μM; “+++” means IC₅₀ between 0.05 and 0.5 μM; “++” means IC₅₀ between 0.5 and 5 μM, “+” means IC₅₀ between 5 and 50 μM and “nd” means “not determined yet”.

The present invention encompasses the compounds of formula I to XXXIV as well as all those listed in Tables 1, 2, and 3 as well as stereoisomers, tautomers, racemates, prodrugs, metabolites thereof, or a pharmaceutically acceptable salt and/or solvate thereof.

TABLE 1

| | | IC₅₀ |
|---|---|---|
| Name | # Compound | ROCK |
| 4-(1-Amino-ethyl)-3-hydroxy-N-pyridin-4-yl-benzamide | 1 | ++ |
| 4-(1-Amino-ethyl)-3-nitro-N-pyridin-4-yl-benzamide | 2 | +++ |
| 3-Amino-4-(1-amino-ethyl)-N-pyridin-4-yl-benzamide | 3 | ++ |
| 6-(1-Amino-ethyl)-4′-chloro-biphenyl-3-carboxylic acid pyridin-4- | 4 | ++++ |
| ylamide | | |
| 4-(1-Amino-ethyl)-N-pyridin-4-yl-3-thiophen-2-yl-benzamide | 5 | ++++ |
| 6-(1-Amino-ethyl)-4′-methoxy-biphenyl-3-carboxylic acid | 6 | +++ |
| pyridin-4-ylamide | | |
| 4-(1-Amino-ethyl)-3-furan-3-yl-N-pyridin-4-yl-benzamide | 7 | +++ |
| 4-(1-Amino-ethyl)-3-(2-hydroxy-ethoxy)-N-pyridin-4-yl- | 8 | ++ |
| benzamide | | |
| 4-(1-Amino-ethyl)-3-(3-hydroxy-propoxy)-N-pyridin-4-yl- | 9 | ++ |
| benzamide | | |
| 3-(2-Amino-ethoxy)-4-(1-amino-ethyl)-N-pyridin-4-yl-benzamide | 10 | ++ |
| 4-(1-Amino-ethyl)-3-(3-amino-propyl)-N-pyridin-4-yl-benzamide | 11 | ++ |
| 4-(1-Amino-ethyl)-3-benzyloxy-N-pyridin-4-yl-benzamide | 12 | +++ |
| 4-(1-Amino-ethyl)-3-(3-phenyl-thioureido)-N-pyridin-4-yl- | 13 | +++ |
| benzamide | | |
| 4-(1-Amino-ethyl)-3-(3-phenyl-ureido)-N-pyridin-4-yl-benzamide | 14 | +++ |
| 4-(1-Amino-ethyl)-3-benzylamino-N-pyridin-4-yl-benzamide | 15 | nd |
| 4-(1-Amino-ethyl)-3-[(furan-2-ylmethyl)-amino]-N-pyridin-4-yl- | 16 | +++ |
| benzamide | | |
| 4-(1-Amino-ethyl)-3-benzoylamino-N-pyridin-4-yl-benzamide | 17 | +++ |
| Furan-2-carboxylic acid [2-(1-amino-ethyl)-5-(pyridin-4- | 18 | +++ |
| ylcarbamoyl)-phenyl]-amide | | |
| 4-(1-Amino-ethyl)-3-(3-methoxy-benzoylamino)-N-pyridin-4-yl- | 19 | ++++ |
| benzamide | | |
| 4-(1-Amino-ethyl)-3-(3-chloro-benzoylamino)-N-pyridin-4-yl- | 20 | +++ |
| benzamide | | |
| 4-(1-Amino-ethyl)-3-(4-methoxy-benzoylamino)-N-pyridin-4-yl- | 21 | ++++ |
| benzamide | | |
| 4-(1-Amino-ethyl)-3-(4-chloro-benzoylamino)-N-pyridin-4-yl- | 22 | +++ |
| benzamide | | |

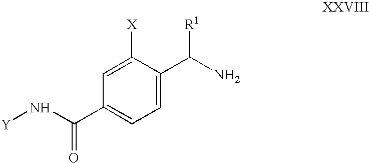

TABLE 2

| Compound | | | | | IC₅₀ |
|---|---|---|---|---|---|
| # | Y | X | R¹ | Name | ROCK |
| 23 | 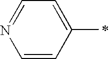 | 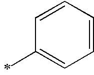 | *—CH₃ | 6-(1-aminoethyl)-N-pyridin-4-ylbiphenyl-3- carboxamide | nd |
| 24 | 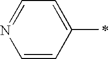 | 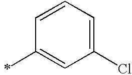 | *—CH₃ | 6-(1-aminoethyl)-3′-chloro-N-pyridin-4- ylbiphenyl-3-carboxamide | ++++ |
| 25 | 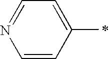 | 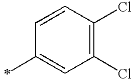 | *—CH₃ | 6-(1-aminoethyl)-3′,4′-dichloro-N-pyridin-4- ylbiphenyl-3-carboxamide | nd |
| 26 | 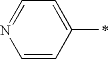 | 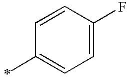 | *—CH₃ | 6-(1-aminoethyl)-4′-fluoro-N-pyridin-4- ylbiphenyl-3-carboxamide | ++++ |
| 27 | 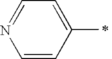 | 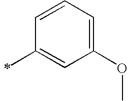 | *—CH₃ | 6-(1-aminoethyl)-3′-methoxy-N-pyridin-4- ylbiphenyl-3-carboxamide | nd |
| 28 | 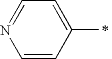 | 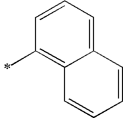 | *—CH₃ | 4-(1-aminoethyl)-3-(5,8- dihydronaphthalen-1-yl)-N-pyridin-4- ylbenzamide | nd |
| 29 | 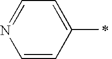 | 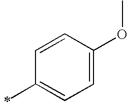 | *—CH₃ | 6-(1-aminoethyl)-4′-methoxy-N-pyridin-4- ylbiphenyl-3-carboxamide | nd |
| 30 | 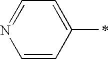 | 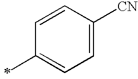 | *—CH₃ | 6-(1-aminoethyl)-4′-cyano-N-pyridin-4- ylbiphenyl-3-carboxamide | nd |
| 31 | 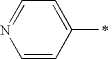 | 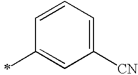 | *—CH₃ | 4-(1-aminoethyl)-3′-cyano-N-pyridin-4- ylbiphenyl-3-carboxamide | nd |
| 32 | 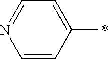 | 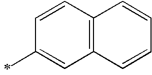 | *—CH₃ | 4-(1-aminoethyl)-3-(2-naphthyl)-N-pyridin- 4-ylbenzamide | ++++ |
| 33 | 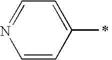 | 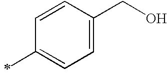 | *—CH₃ | 6-(1-aminoethyl)-4′-(hydroxymethyl)-N- pyridin-4-ylbiphenyl-3-carboxamide | nd |
| 34 | 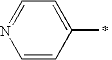 | 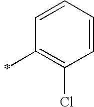 | *—CH₃ | 6-(1-aminoethyl)-2′-chloro-N-pyridin-4- ylbiphenyl-3-carboxamide | nd |
| 35 | 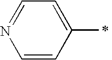 | 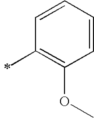 | *—CH₃ | 6-(1-aminoethyl)-2′-methoxy-N-pyridin-4- ylbiphenyl-3-carboxamide | nd |
| 36 | 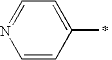 | 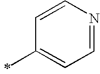 | *—CH₃ | 4-(1-aminoethyl)-N,3-dipyridin-4- ylbenzamide | nd |
| 37 | 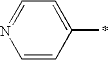 | 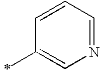 | *—CH₃ | 4-(1-aminoethyl)-3-pyridin-3-yl-N-pyridin- 4-ylbenzamide | nd |
| 38 | 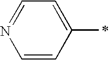 | 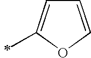 | *—CH₃ | 4-(1-aminoethyl)-3-(2-furyl)-N-pyridin-4- ylbenzamide | nd |
| 39 | 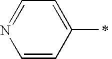 | 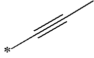 | *—CH₃ | 4-(1-aminoethyl)-3-(phenylethynyl)-N- pyridin-4-ylbenzamide | nd |
| 40 | 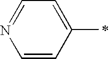 | 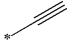 | *—CH₃ | 4-(1-aminoethyl)-N-pyridin-4-yl-3-(pyridin- 2-ylethynyl)benzamide | nd |
| 41 | 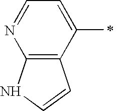 | 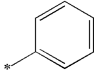 | *—CH₃ | 6-(1-aminoethyl)-N-1H-pyrrolo[2,3- b]pyridin-4-ylbiphenyl-3-carboxamide | nd |
| 42 | 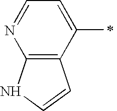 | 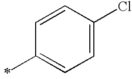 | *—CH₃ | 6-(1-aminoethyl)-4′-chloro-N-1H- pyrrolo[2,3-b]pyridin-4-ylbiphenyl-3- carboxamide | nd |
| 43 | 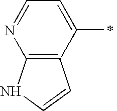 | 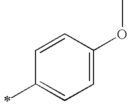 | *—CH₃ | 6-(1-aminoethyl)-4′-methoxy-N-1H- pyrrolo[2,3-b]pyridin-4-ylbiphenyl-3- carboxamide | nd |
| 44 | 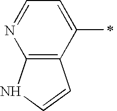 | 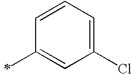 | *—CH₃ | 6-(1-aminoethyl)-3′-chloro-N-1H- pyrrolo[2,3-b]pyridin-4-ylbiphenyl-3- carboxamide | nd |
| 45 | 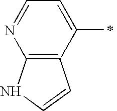 | 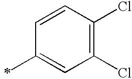 | *—CH₃ | 6-(1-aminoethyl)-3,′,4′-dichloro-N-1H- pyrrolo[2,3-b]pyridin-4-ylbiphenyl-3- carboxamide | nd |
| 46 | 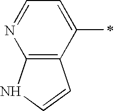 | 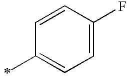 | *—CH₃ | 6-(1-aminoethyl)-4′-fluoro-N-1H- pyrrolo[2,3-b]pyridin-4-ylbiphenyl-3- carboxamide | nd |
| 47 | 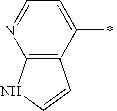 | 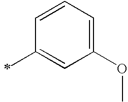 | *—CH₃ | 6-(1-aminoethyl)-3′-methoxy-N-1H- pyrrolo[2,3-b]pyridin-4-ylbiphenyl-3- carboxamide | nd |
| 48 | 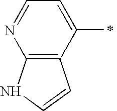 | 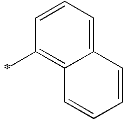 | *—CH₃ | 4-(1-aminoethyl)-3-(5,8- dihydronaphthalen-1-yl)-N-1H-pyrrolo[2,3- b]-pyridin-4-ylbenzamide | nd |
| 49 | 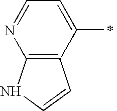 | 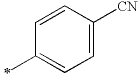 | *—CH₃ | 6-(1-aminoethyl)-4′-cyano-N-1H- pyrrolo[2,3-b]-pyridin-4-ylbiphenyl-3- carboxamide | nd |
| 50 | 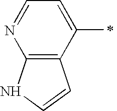 | 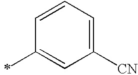 | *—CH₃ | 6-(1-aminoethyl)-3′-cyano-N-1H- pyrrolo[2,3-b]-pyridin-4-ylbiphenyl-3- carboxamide | nd |
| 51 | 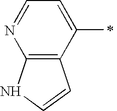 | 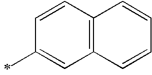 | *—CH₃ | 4-(1-aminoethyl)-3-(2-naphthyl)-N-1H- pyrrolo[2,3-b]-pyridin-4-ylbenzamide | nd |
| 52 | 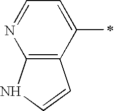 | 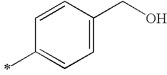 | *—CH₃ | 6-(1-aminoethyl)-4′-(hydroxymethyl)-N-1H- pyrrolo[2,3-b]-pyridin-4-ylbiphenyl-3- carboxamide | nd |
| 53 | 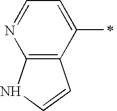 | 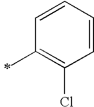 | *—CH₃ | 6-(1-aminoethyl)-2′-chloro-N-1H- pyrrolo[2,3-b]-pyridin-4-ylbiphenyl-3- carboxamide | nd |
| 54 | 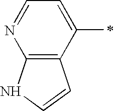 | 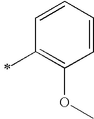 | *—CH₃ | 6-(1-aminoethyl)-2′-methoxy-N-1H- pyrrolo[2,3-b]-pyridin-4-ylbiphenyl-3- carboxamide | nd |
| 55 | 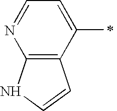 | 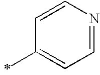 | *—CH₃ | 4-(1-aminoethyl)-3-pyridin-4-yl-N-1H- pyrrolo[2,3-b]pyridin-4-ylbenzamide | nd |
| 56 | 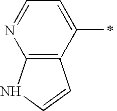 | 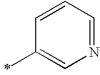 | *—CH₃ | 4-(1-aminoethyl)-3-pyridin-3-yl-N-1H- pyrrolo[2,3-b]pyridin-4-ylbenzamide | nd |
| 57 | 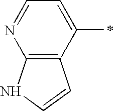 | 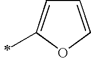 | *—CH₃ | 4-(1-aminoethyl)-3-(2-furyl)-N-1H- pyrrolo[2,3-b]pyridin-4-ylbenzamide | nd |
| 58 | 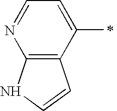 | 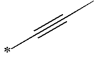 | *—CH₃ | 4-(1-aminoethyl)-3-(phenylethynyl)-N-1H- pyrrolo[2,3-b]pyridin-4-ylbenzamide | nd |
| 59 | 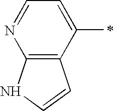 | 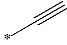 | *—CH₃ | 4-(1-aminoethyl)-3-(pyridin-2-ylethynyl)-N- 1H-pyrrolo[2,3-b]pyridin-4-ylbenzamide | nd |
| 60 | 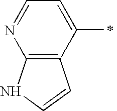 | 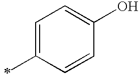 | *—CH₃ | 6-(1-aminoethyl)-4′-hydroxy-N-1H- pyrrolo[2,3-b]pyridin-4-ylbiphenyl-3- carboxamide | nd |
| 61 | 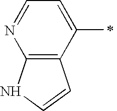 | 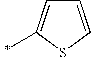 | *—CH₃ | 4-(1-aminoethyl)-N-1H-pyrrolo[2,3- b]pyridin-4-yl-3-(2-thienyl)benzamide | nd |
| 62 | 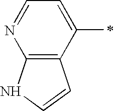 | 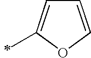 | *—CH₃ | 4-(1-aminoethyl)-3-(3-furyl)-N-1H- pyrrolo[2,3-b]pyridin-4-ylbenzamide | nd |
| 63 | 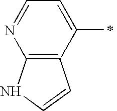 | 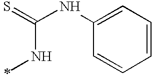 | *—CH₃ | 4-(1-aminoethyl)-3- [(anilinocarbonothioyl)amino]-N-1H- pyrrolo[2,3-b]pyridin-4-ylbenzamide | nd |
| 64 | 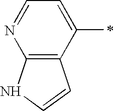 | 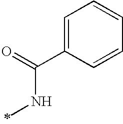 | *—CH₃ | 4-(1-aminoethyl)-3-(benzoylamino)-N-1H- pyrrolo[2,3-b]pyridin-4-ylbenzamide | nd |
| 65 | 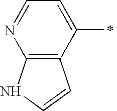 | 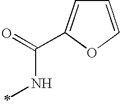 | *—CH₃ | N-{2-(1-aminoethyl)-5-[(1H-pyrrolo[2,3- b]pyridin-4-ylamino)carbonyl]phenyl}-2- furamide | nd |
| 66 | 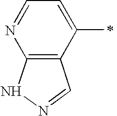 | 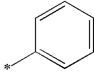 | *—CH₃ | 6-(1-aminoethyl)-N-1H-pyrazolo[3,4- b]pyridin-4-ylbiphenyl-3-carboxamide | nd |
| 67 | 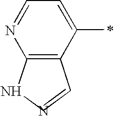 | 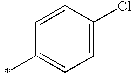 | *—CH₃ | 6-(1-aminoethyl)-4′-chloro-N-1H- pyrazolo[3,4-b]pyridin-4-ylbiphenyl-3- carboxamide | nd |
| 68 | 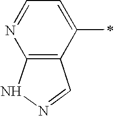 | 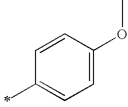 | *—CH₃ | 6-(1-aminoethyl)-4′-methoxy-N-1H- pyrazolo[3,4-b]pyridin-4-ylbiphenyl-3- carboxamide | nd |
| 69 | 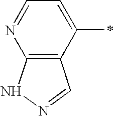 | 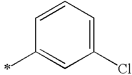 | *—CH₃ | 6-(1-aminoethyl)-3′-chloro-N-1H- pyrazolo[3,4-b]pyridin-4-ylbiphenyl-3- carboxamide | nd |
| 70 | 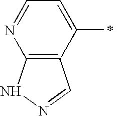 | 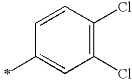 | *—CH₃ | 6-(1-aminoethyl)-3′,4′-dichloro-N-1H- pyrazolo[3,4-b]pyridin-4-ylbiphenyl-3- carboxamide | nd |
| 71 | 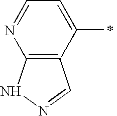 | 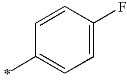 | *—CH₃ | 6-(1-aminoethyl)-4′-fluoro-N-1H- pyrazolo[3,4-b]pyridin-4-ylbiphenyl-3- carboxamide | nd |
| 72 | 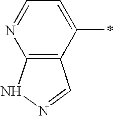 | 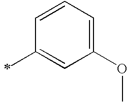 | *—CH₃ | 6-(1-aminoethyl)-3′-methoxy-N-1H- pyrazolo[3,4-b]pyridin-4-ylbiphenyl-3- carboxamide | nd |
| 73 | 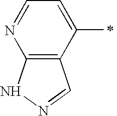 | 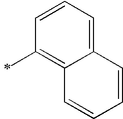 | *—CH₃ | 4-(1-aminoethyl)-3-(5,8- dihydronaphthalen-1-yl)-N-1H- pyrazolo[3,4-b]-pyridin-4-ylbenzamide | nd |
| 74 | 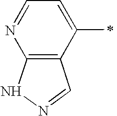 | 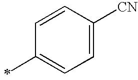 | *—CH₃ | 6-(1-aminoethyl)-4′-cyano-N-1H- pyrazolo[3,4-b]-pyridin-4-ylbiphenyl-3- carboxamide | nd |
| 75 | 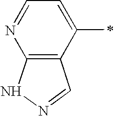 | 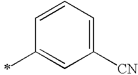 | *—CH₃ | 6-(1-aminoethyl)-3′-cyano-N-1H- pyrazolo[3,4-b]-pyridin-4-ylbiphenyl-3- carboxamide | nd |
| 76 | 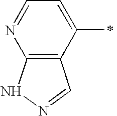 | 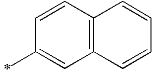 | *—CH₃ | 4-(1-aminoethyl)-3-(2-naphthyl)-N-1H- pyrazolo[3,4-b]-pyridin-4-ylbenzamide | nd |
| 77 | 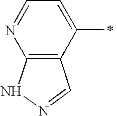 | 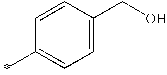 | *—CH₃ | 6-(1-aminoethyl)-4′-(hydroxymethyl)-N-1H- pyrazolo[3,4-b]pyridin-4-ylbiphenyl-3- carboxamide | nd |
| 78 | 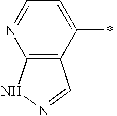 | 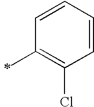 | *—CH₃ | 6-(1-aminoethyl)-2′-chloro-N-1H- pyrazolo[3,4-b]-pyridin-4-ylbiphenyl-3- carboxamide | nd |
| 79 | 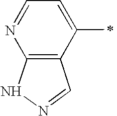 | 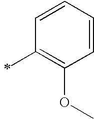 | *—CH₃ | 6-(1-aminoethyl)-2′-methoxy-N-1H- pyrazolo[3,4-b]-pyridin-4-ylbiphenyl-3- carboxamide | nd |
| 80 | 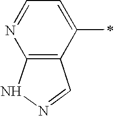 | 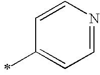 | *—CH₃ | 4-(1-aminoethyl)-3-pyridin-4-yl-N-1H- pyrazolo[3,4-b]pyridin-4-ylbenzamide | nd |
| 81 | 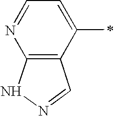 | 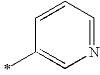 | *—CH₃ | 4-(1-aminoethyl)-3-pyridin-3-yl-N-1H- pyrazolo[3,4-b]pyridin-4-ylbenzamide | nd |
| 82 | 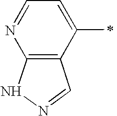 | 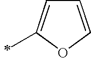 | *—CH₃ | 4-(1-aminoethyl)-3-(2-furyl)-N-1H- pyrazolo[3,4-b]pyridin-4-ylbenzamide | nd |
| 83 | 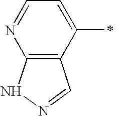 | 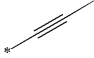 | *—CH₃ | 4-(1-aminoethyl)-3-(phenylethynyl)-N-1H- pyrazolo[3,4-b]pyridin-4-ylbenzamide | nd |
| 84 | 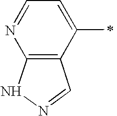 | 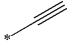 | *—CH₃ | 4-(1-aminoethyl)-3-(pyridin-2-ylethynyl)-N- 1H-pyrazolo[3,4-b]pyridin-4-ylbenzamide | nd |
| 85 | 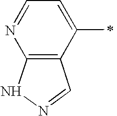 | 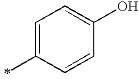 | *—CH₃ | 6-(1-aminoethyl)-4′-hydroxy-N-1H- pyrazolo[3,4-b]pyridin-4-ylbiphenyl-3- carboxamide | nd |
| 86 | 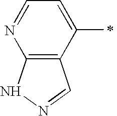 | 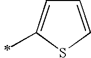 | *—CH₃ | 4-(1-aminoethyl)-N-1H-pyrazolo[3,4- b]pyridin-4-yl-3-(2-thienyl)benzamide | nd |
| 87 | 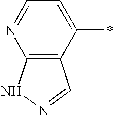 | 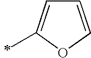 | *—CH₃ | 4-(1-aminoethyl)-3-(3-furyl)-N-1H- pyrazolo[3,4-b]pyridin-4-ylbenzamide | nd |
| 88 | 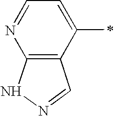 | 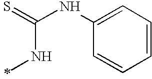 | *—CH₃ | 4-(1-aminoethyl)-3- [(anilinocarbonothioyl)amino]-N-1H- pyrazolo[3,4-b]pyridin-4-ylbenzamide | nd |
| 89 | 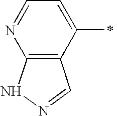 | 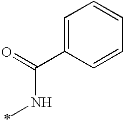 | *—CH₃ | 4-(1-aminoethyl)-3-(benzoylamino)-N-1H- pyrazolo[3,4-b]pyridin-4-ylbenzamide | nd |
| 90 | 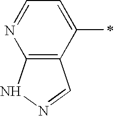 | 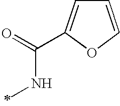 | *—CH₃ | N-{2-(1-aminoethyl)-5-[(1H-pyrazolo[3,4- b]pyridin-4-ylamino)carbonyl]phenyl}-2- furamide | nd |
| 91 | 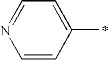 | 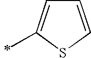 | *—Et | 4-((R)-1-amino-propyl)-N-pyridin-4-yl-3- thiophen-2-yl-benzamide | nd |
| 92 | 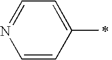 | 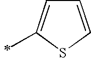 | *—Et | 4-((S)-1-amino-propyl)-N-pyridin-4-yl-3- thiophen-2-yl-benzamide | nd |
| 93 | 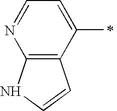 | 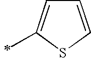 | *—Et | 4-((R)-1-amino-propyl)-N-(1H-pyrrolo[2,3- b]pyridin-4-yl)-3-thiophen-2-yl-benzamide | nd |
| 94 | 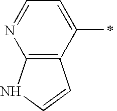 | 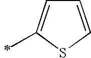 | *—Et | 4-((S)-1-amino-propyl)-N-(1H-pyrrolo[2,3- b]pyridin-4-yl)-3-thiophen-2-yl-benzamide | nd |
| 95 | 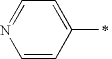 | 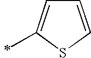 | 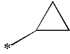 | 4-((R)-amino-cyclopropyl-methyl)-N- pyridin-4-yl-3-thiophen-2-yl-benzamide | nd |
| 96 | 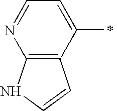 | 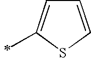 | 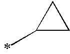 | 4-((R)-amino-cyclopropyl-methyl)-N-(1H- pyrrolo[2,3-b]pyridin-4-yl)-3-thiophen-2-yl- benzamide | nd |
| 97 | 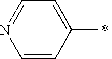 | 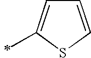 | 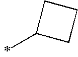 | 4-((R)-amino-cyclobutyl-methyl)-N-pyridin- 4-yl-3-thiophen-2-yl-benzamide | nd |
| 98 | 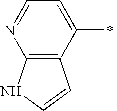 | 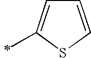 | 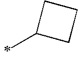 | 4-((R)-amino-cyclobutyl-methyl)-N-(1H- pyrrolo[2,3-b]pyridin-4-yl)-3-thiophen-2-yl- benzamide | nd |
| 99 | 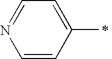 | 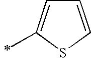 | 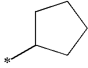 | 4-((R)-amino-cyclopentyl-methyl)-N- pyridin-4-yl-3-thiophen-2-yl-benzamide | nd |
| 100 | 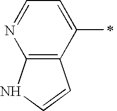 | 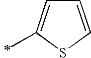 | 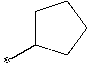 | 4-((R)-amino-cyclopentyl-methyl)-N-(1H- pyrrolo[2,3-b]pyridin-4-yl)-3-thiophen-2-yl- benzamide | nd |
| 101 | 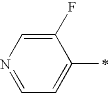 | 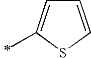 | *—CH₃ | 4-((R)-1-amino-ethyl)-N-(3-fluoro-pyridin- 4-yl)-3-thiophen-2-yl-benzamide | nd |
| 102 | 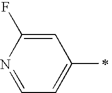 | 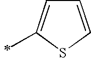 | *—CH₃ | 4-((R)-1-amino-ethyl)-N-(2-fluoro-pyridin- 4-yl)-3-thiophen-2-yl-benzamide | nd |

### Compound 103: 4-((R)-1-Amino-ethyl)-N-pyridin-4-yl-3-thiophen-2-yl-benzamide dihydrochloric acid salt

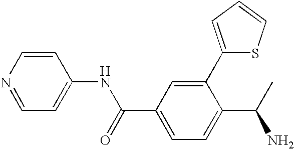

Compound 103 was obtained by preparative chiral HPLC of compound 5 (Column: AD-H, 10×250 mm, 5 μm; Solvent: hexane/ethanol 90/10 with 0.1% DIPEA).

% ee=100% (chiral HPLC: column AD-H, 0.46×250 mm, hexane/ethanol 90/10 with 0.1% DIPEA, Tᵣₑₜ: 38.3 min).

### Compound 104: 4-((S)-1-Amino-ethyl)-N-pyridin-4-yl-3-thiophen-2-yl-benzamide dihydrochloric acid salt

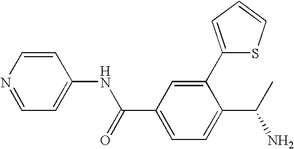

Compound 104 was obtained by preparative chiral HPLC of compound 5 (Column: AD-H, 10×250 mm, 5 μm; Solvent: hexane/ethanol 90/10 with 0.1% DIPEA).

% ee=99.8% (chiral HPLC: column AD-H, 0.46×250 mm, hexane/ethanol 90/10 with 0.1% DIPEA, Tᵣₑₜ: 53.8 min).

TABLE 3

| | | IC₅₀ |
|---|---|---|
| Name | # Compound | ROCK |
| 4-((R)-1-Amino-ethyl)-N-pyridin-4- | 103 | ++++ |
| yl-3-thiophen-2-yl-benzamide | | |
| 4-((S)-1-Amino-ethyl)-N-pyridin-4- | 104 | +++ |
| yl-3-thiophen-2-yl-benzamide | | |

### Example 6

### Biochemical Profile

### Potency and Selectivity in Initial Kinase Panel

Potency and selectivity of compounds of the invention to closely related kinases was evaluated (data not shown). Compared with known ROCK inhibitors such as Y-27632, compounds of the invention are at least as potent or 10× more potent and have improved selectivity versus closely related protein kinases of the AGC-family such as, PKA, PKB and PKC.

### Specificity

Compounds of the invention were also tested for specificity on a panel of 21 receptors, ion channels and transporters at 10 μM (data not shown). For the specificity panel, representative sets of neurotransmitter- and hormone receptors, ion channels and neurotransmitter transporters were selected. The data obtained with this panel supported a good specificity with the potential for minimal pharmacological side-effects of the profiled compounds.

It was shown that compounds of the invention are potent, selective and specific ROCK inhibitor.

### Cell Based Profile

Kinase inhibitors generally show a loss in potency between biochemical assays and cellular assays due to cell penetration and differences in ATP concentrations. To have a more accurate perspective on the selectivity of the present ROCK inhibitors, the in vitro biochemical data of compounds of the invention were complemented with in vitro cellular IC50-values.

A cellular assay was configured measuring ROCK activity in a physiological context. The assay is based on the observation that lipopolysaccharide (LPS) induced TNFα release from human monocytes/macrophages is in part dependent on ROCK.

The data (not shown) demonstrate that compounds of the invention have an in vitro cellular IC₅₀ which is similar or at least 10-20 fold more potent than prior art compounds.

eADME-tox Profile

The most relevant parameters for predicting human pharmacokinetics and toxicity have been established.

Compounds of the invention show improved and attractive eADME-tox properties (data not shown).

Among these properties are: high solubility, medium to high permeability, required metabolic stability, no in vitro toxicity. These properties allow both systemic and topical application.

Compounds of the invention were also tested for potential effects on cell viability and cytotoxicity.

Cell viability and cytotoxicity were assessed in human embryonic kidney cells (HEK293T). HEK293T cells were incubated for 48 hours in the presence of compound. At the end of the incubation period, cell viability was assessed by quantification of the ATP content (CellTiter Glo, Promega) and the supernatant was assayed for presence of LDH (CytoTox One, Promega), both of which serve as a markers for cytotoxicity. Up to concentrations of 30 μM, no effects on cell viability and cytotoxicity were observed for any of compounds of the invention.

### PK Profile

The pharmacokinetic parameters of compounds of the invention were studied in male CD-1 mice and in male Sprague-Dawley rats using cassette administration (Data not shown). No adverse reactions were observed during administration of compounds of the invention.

The overall systemic exposure translated into very good absolute bioavailability. The systemic levels of compounds are consistent with achieving pharmacological enzyme inhibition for several hours post oral administration.

The data described above illustrate that the compounds of the present invention are potent, selective and specific ROCK inhibitors with very favorable eADME, Tox and PK-properties.

All patents, patent applications, and published references cited herein are hereby incorporated by reference in their entirety. While this invention has been particularly shown and described with references to preferred embodiments, it will be understood by those skilled in the art that various changes in form and details may be made without departing from the scope of the invention encompassed by the claims.

### Example 7

### Biological Activity

### Activity on Hypertension:

The efficiency of compounds of the invention to reduce and/or treat hypertension in an acute model was evaluated using spontaneous hypertensive rats (SHRs).

Vehicle, clonidine (0.3 mg/kg) positive control, Y-27632 reference compound (10 mg/kg), an example compound (3,10 and 30 mg/kg) falling under formula X, were administered orally.

Compounds were administered as one single dose to SHR. Systolic arterial pressure (SAP) and heart rate were recorded before (−1 hr) and 1, 3, 5, 7, and 24 hours after oral administration of vehicle or test compound.

A reduction in SAP by 10 percent or more (>10%), or a decrease in heart rate by 20 percent or more (>20%), relative to baseline was considered significant.

### Results:

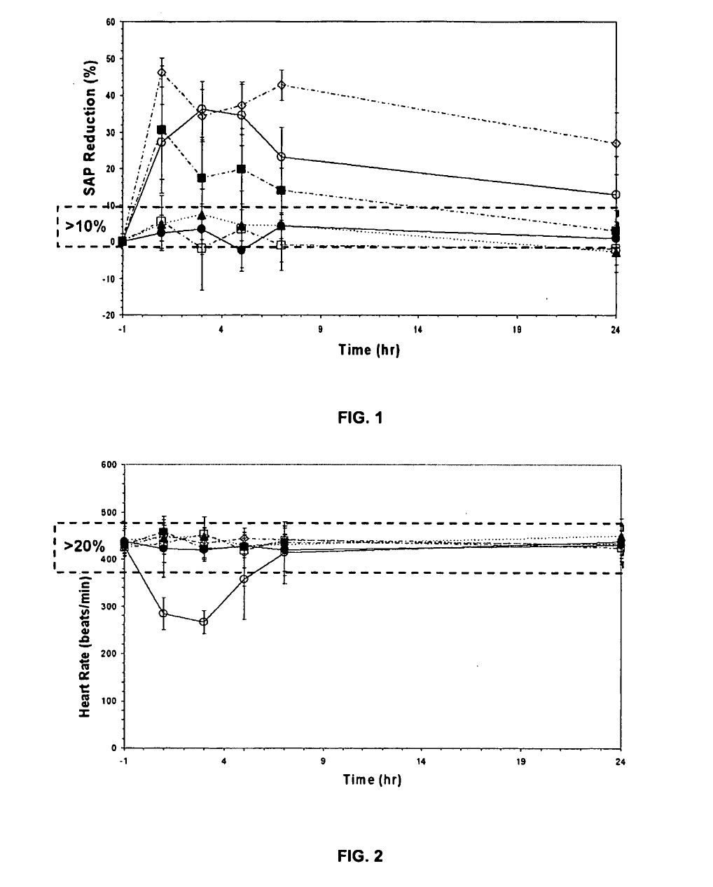

As illustrated in FIG. 1, it is clear that the compound falling under formula X both at 10 mg/kg (▪) and 30 mg/kg (⋄), caused a significant and very profound blood pressure lowering effect, respectively 1-7 hours and 1-24 hours post dosing.

The hypotensive response of Y-27632 reference compound (▴) at 10 mg/kg, contrary to what is described in literature (Uehata et al. Nature 1997, 389, 990.), was not significant.

None of the compounds tested had an effect on heart rate (See FIG. 2). This indicates that the blood pressure lowering observed with compounds of the invention, is not caused (fully or partially like clonidine) by modulation of the heart rate.

### Conclusion:

Compounds of the invention have significant hypotensive activity without affecting heart rate, whereas Y-27632 (10 mg/kg) is completely inactive in the present SHR setup.

### In Vivo Biomarker Assay

ROCK plays a role in LPS induced cytokine production and especially TNFα.

The in vivo effects of compounds according to the invention are investigated.

Male mice are dosed with 30 mg/kg of compound of the invention or vehicle via PO or IP routes. Following 2 h and 4 h post dose mice are challenged with LPS and a blood sample is taken 1 h after the LPS challenge to investigate the blood TNFα level using off the shelf technology.

The results will corroborate the fact that the compound of the invention is capable of suppressing the TNFα release efficiently after IP and PO dosing.

### Carrageenan Model of Inflammation

The efficiency of compounds of the invention to reduce acute inflammation is evaluated using the Carrageenan model: 5-6 weeks old Swiss Webster mice (Harlan) are weighed and the right paw volume measured by water displacement at the start of the experiment. The animals (n=10) are administered with either vehicle or 10 mg/kg 30 mg/kg of a compound of the invention orally, 2 h before carrageenan injection into the paw. Two hours after oral administration animals are anesthetized and injected with 50 μl (10 mg/ml) carrageenan in the sub plantar region of the paw. Two, four and six hours following the injection, the paw volumes are measured.

A reduction in paw volume is seen with both 10 mg/kg and 30 mg/kg of a compound of the invention. The two-hour value has a significance of >99% according to the T test.

The results will corroborate the fact that the compound of the invention is capable of suppressing the inflammatory response in the Carrageenan model with 10 mg/kg being a dose with maximal effect.

### Efficacy of Anti-Inflammatory Activity in a Mouse Model for Inflammatory Bowel Disease

The efficiency of compounds of the invention to reduce and/or treat hypertension in an acute model was evaluated using spontaneous hypertensive rats (SHRs).

Inflammatory bowel disease (IBD), is a chronic, disabling, and often relapsing autoimmune disorder associated with high morbidity. IBD is composed of a group of chronic inflammatory illnesses of the gastrointestinal tract which mainly affect young adults. These diseases are characterized by frequent episodes of diarrhea, abdominal pain, blood in the stool, and weight loss over a period of months to years; in some instances the deterioration of the intestinal tract requires surgical intervention, such as bowel resection.

The purpose of this example is to administer test compounds and assess anti-inflammatory activity in a mouse model of Inflammatory Bowel Disease (IBD). Each test compound is dosed at 3, 10 and 30 mg/kg. The group size for each dose is 10 mice.

160 C57BI/6 mice (female, 5-6 weeks old) are received in quarantine. Following quarantine, random groups of 10 mice per group are select for the study and assigned. At day 0, the mice are food deprived for 24 hours. Dosing with vehicle, positive control and test compounds is started twice daily. The first dose is administered 1 hour prior to trinitrobenzene sulfonate (TNBS) challenge. Individual mouse weights are recorded. Mice are anesthetized and enema tubing (4 cm PE-20 tubing fitted with 25 G needle) is inserted into the mouse anus. The TNBS solution (50% TNBS 50% 200 proof ethanol) is administered into the colon of each mouse whereafter the tubing is removed and the anus is hold closed. The mice are maintained in head down position until recovery from anesthesia. The mice are returned to routine housing with access to food and water

The dosing schedule is at follows:

a) Twice daily at 12-hour intervals on all days beginning at DAY 0 and continuing until administration of a single dose on DAY 6. Once daily for positive control:prednisolone.
b) DAY 0: Daily dosing starts.
c) DAY 0: First dose at 1 hour prior to TNBS challenge
d) DAY 0: Second dose 12 hours later
e) DAYS 1-5: Continued twice-daily dosing
f) DAY 6: Dose once 3 hours prior to euthanization

The test compounds are administered as follows:

a) Animals are dosed twice daily by oral gavage (1 ml/kg) with vehicle or test compound.
b) Animals in positive control group (prednisolone 10 mg/kg) are dosed once daily by oral gavage.
c) The vehicle and test compounds are dosed twice daily from DAY 0 to DAY 5 and once on DAY 6.

At DAY 6, 3 hours after final dosing, the mice are euthanized, the colon is removed, a longitudinal incision is made therein and the colon is gently washed with saline to remove fecal material. The severity of IBD using the following scoring system:
maximum score=10
no damage=0
hyperemia without ulcers=1
hyperemia and thickening of the bowel wall without ulcers=2
one ulcer without thickening of the bowel wall=3
two sites of ulceration or inflammation=4
more than two sites of inflammation or 1 site extending over 0.5 cm=5
damage extending at least 1 cm=6
damage extending at least 1.5 cm=7
damage extending at least 2 cm=8
damage extending at least 2.5 cm=9
damage extending at least 3 cm=10

The data described above corroborate the fact that the compound of the present invention is capable of selectively and specifically inhibit ROCK with very favorable eADME, Tox and PK-properties.

In conclusion, the compounds of the invention are particularly suitable as medicament and for the treatment of:

VSMC hypercontraction including but not limited to cerebral vasospasm, coronary vasospasm, hypertension, pulmonary hypertension, and sudden death,

Other SMC disorders including but not limited to bronchial asthma and glaucoma,

Arteriosclerotic diseases including but not limited to angina, myocardial infraction, restenosis, stroke, hypertensive vascular disease, heart failure, cardiac allograft vasculopathy and vein graft disease,

Other disorder including but not limited to osteoporosis, erectile dysfunction and cancers (such as metastasis, cell migration for example), spinal-cord injury, stroke, HIV, inflammatory and demyelinising diseases, Alzheimer's disease, neuropathic pain, asthma, pre-term labor, renal disease.

The present invention encompasses compounds 1 to 104 and stereoisomers, tautomers, racemics or a pharmaceutically acceptable salt and/or solvate thereof.

The present invention also encompasses methods for assigning a function in inflammation to a ROCK inhibitor, comprising the steps of: providing a ROCK inhibitor, testing the activity of said inhibitor on at least one parameter selected from:

(i) a LPS induced TNF release,
(ii) a carrageenan induced edema model,
and determining from a positive outcome of said at least one parameter the use of the ROCK inhibitor for preventing, alleviating, treating a condition or a disease related to inflammation.

The present invention thus also encompasses a method for assigning a function in inflammation to a compound, comprising the steps of: providing a compound, testing the activity of said compound on an in vitro or in vivo ROCK inhibition assay and determining from a positive outcome of said inhibition assay the use of the compound for preventing, alleviating, treating a condition or a disease related to inflammation. The present invention preferably encompasses a method for assigning a function in inflammation to a compound, comprising the steps of: providing a compound, testing activity of said compound on ROCK.

All patents, patent applications, and published references cited herein are hereby incorporated by reference in their entirety. While this invention has been particularly shown and described with references to preferred embodiments, it will be understood by those skilled in the art that various changes in form and details may be made without departing from the scope of the invention encompassed by the claims.

1. A compound of Formula I, II, or III, or a stereoisomer, tautomer, racemate, salt, hydrate, or solvate thereof, 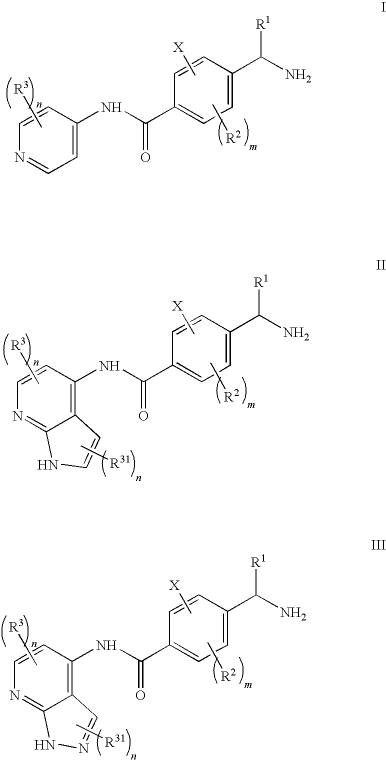
wherein
X is a group selected from hydroxyl, amino, nitro, alkoxy, alkylamino, hydroxyalkyloxy, aminoalkyloxy, alkynyl, arylalkynyl, heteroarylalkynyl, aryl, heteroaryl, aralkyl, heteroarylalkyl, aryloxy, heteroaryloxy, arylalkoxy, arylaminothiocarbonylamino, heteroarylaminothiocarbonylamino, arylalkylamino, heteroarylalkylamino, arylcarbonylamino, heteroarylcarbonylamino, arylaminocarbonyl, heteroarylaminocarbonyl, arylaminocarbonylamino, or heteroarylaminocarbonylamino, each group being optionally substituted by one or more substituents selected from halogen, hydroxyl, oxo, nitro, amino, aminocarbonyl, azido, cyano, carboxyl, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, arylamino, acyl, arylcarbonyl, aminocarbonyl, alkylsulfoxide, —SO₂R¹⁵, or alkylthio, wherein R¹⁵ is alkyl or cycloalkyl,
R¹ is hydrogen or a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
R² is hydrogen, halogen, nitro, cyano, or hydroxyl, or a group selected from alkyl, alkenyl, alkynyl, amino, acyl, acylamino, alkoxy, arylamino, haloalkoxy, aryl, or heteroaryl, each group being optionally substituted by one or more substituents selected from hydroxyl, halogen, oxo, nitro, amino, cyano, alkyl, alkoxy, haloalkyl, or haloalkoxy, and m is an integer m is an integer selected from 0, 1, 2, or 3, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is an integer selected from 0, 1, 2, or 3.

2. A compound according to claim 1, wherein:
X is a group selected from hydroxyl, amino, nitro, alkoxy, alkylamino, hydroxyalkyloxy, aminoalkyloxy, alkynyl, arylalkynyl, heteroarylalkynyl, aryl, heteroaryl, aralkyl, heteroarylalkyl, aryloxy, heteroaryloxy, arylalkoxy, arylaminothiocarbonylamino, heteroarylaminothiocarbonylamino, arylalkylamino, heteroarylalkylamino, arylcarbonylamino, heteroarylcarbonylamino, arylaminocarbonyl, heteroarylaminocarbonyl, arylaminocarbonylamino, or heteroarylaminocarbonylamino, each group being optionally substituted by one or more substituents selected from halogen, hydroxyl, nitro, cyano, amino, alkyl, alkoxy, aryl, arylamino, heteroaryl, haloalkyl, haloalkoxy, heterocyclyl, or aryloxy,
R¹ is a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
R² is hydrogen, halogen, nitro, cyano, or hydroxyl, or a group selected from alkyl, alkenyl, alkynyl, amino, acyl, acylamino, alkoxy, arylamino, haloalkoxy, aryl, or heteroaryl, each group being optionally substituted by one or more substituents selected from hydroxyl, halogen, oxo, nitro, amino, cyano, alkyl, alkoxy, haloalkyl, or haloalkoxy, m is 0, 1, or 2, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, 2 and 3.

3. A compound according to claim 1, having one of the structural Formula IV, V, VI, VII, VIII, IX, X, XI, or XII, wherein: 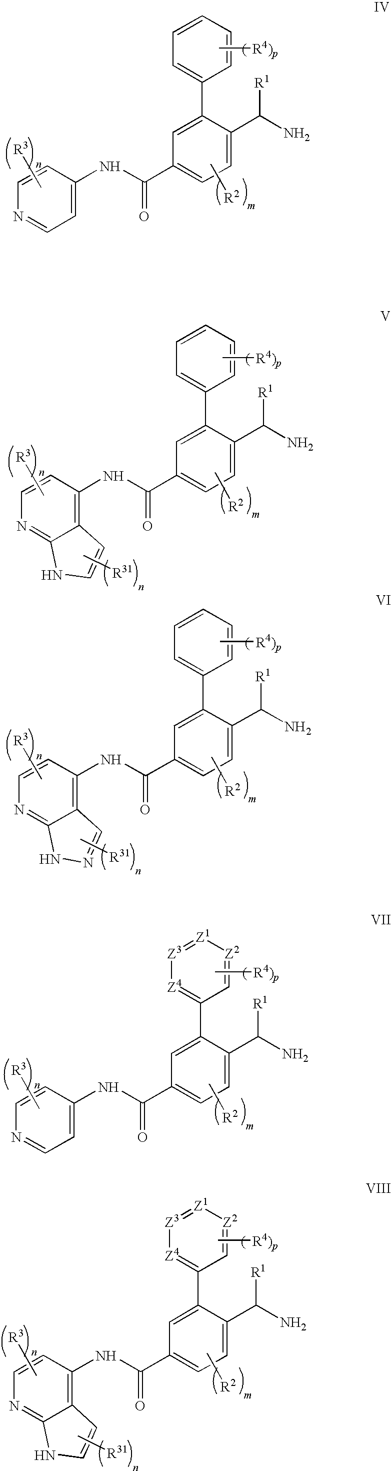 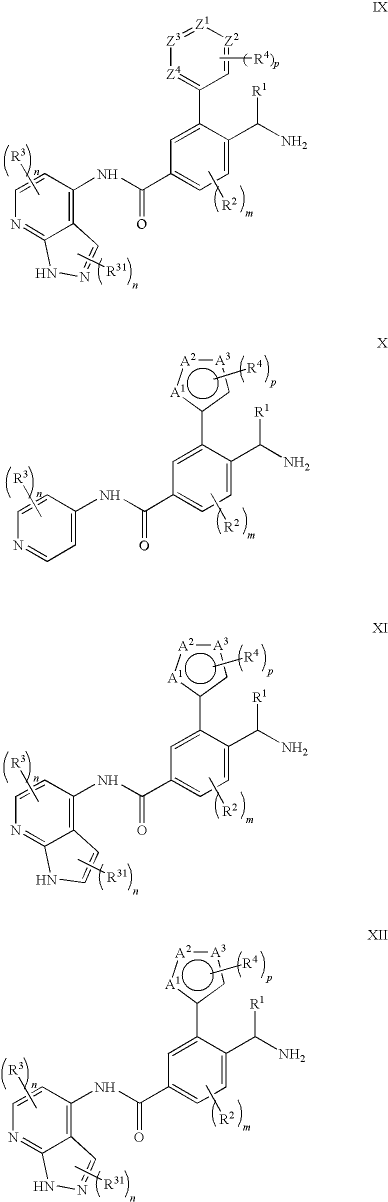
Z¹, Z², Z³, Z⁴ are each independently selected from CH or N, wherein at least one of Z¹, Z², Z³, or Z⁴ is a N atom,
A¹, A², A³ are each independently selected from CH, N, NH, O, or S, wherein at least one of A¹, A², A³ is a heteroatom selected from N, O, or S,
R⁴ is selected from halogen, hydroxyl, nitro, cyano, amino, alkyl, alkoxy, aryl, heteroaryl, haloalkyl, haloalkoxy, heterocyclyl, or aryloxy, p is an integer from 0, 1, 2, 3, 4, or 5, or two R⁴ form together an aryl, heteroaryl, or heterocyclyl fused to the aromatic ring to which they are attached, with p being at least 2, and
R¹, R², R³, R³¹, n, m have the same meaning as that defined in claim 1 or 2.

4. A compound according to claim 3, wherein:
Z¹, Z², Z³, Z⁴ are each independently selected from CH or N, wherein at least one of Z¹, Z², Z³, or Z⁴ is a N atom,
A¹, A², A³ are each independently selected from CH, N, NH, O, or S, wherein at least one of A¹, A², A³ is a heteroatom selected from N, O, or S,
R⁴ is selected from halogen, hydroxyl, nitro, cyano, amino, alkyl, alkoxy, aryl, heteroaryl, haloalkyl, haloalkoxy, heterocyclyl, or aryloxy, p is selected from 0, 1, 2, 3, 4, or 5, or two R⁴ form together an aryl, heteroaryl, or heterocyclyl fused to the aromatic ring to which they are attached with p being at least 2,
R¹ is a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
R² is selected from hydrogen, halogen, nitro, cyano, hydroxyl, alkyl, amino, alkoxy, haloalkoxy, aryl, or heteroaryl, m is 0 or 1, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, or 2.

5. A compound according to claim 1, having one of the structural Formula XIII, XIV, XV, XVI, XVII, XVIII, XIX, XX, XXI, XXII, XXIII, XXIV, XXV, XXVI, or XXVII, wherein: 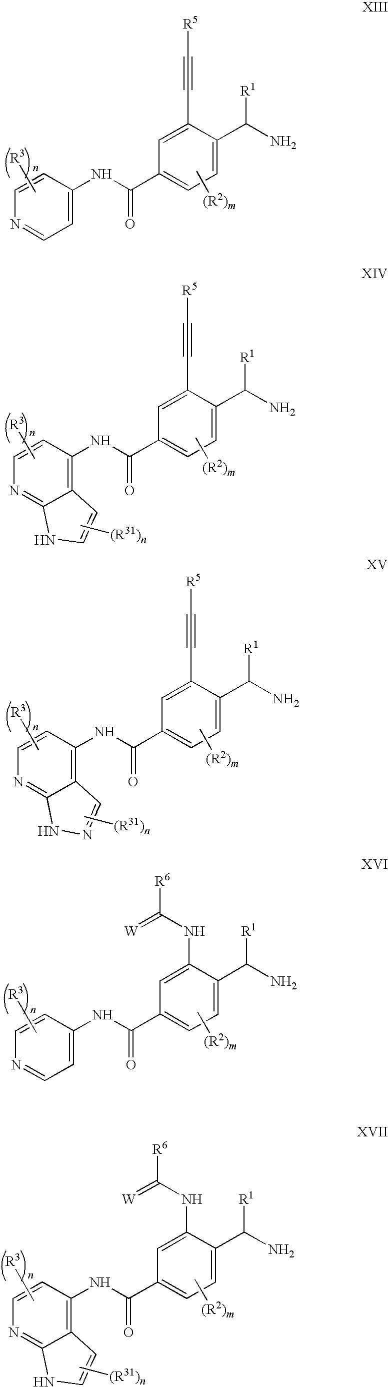 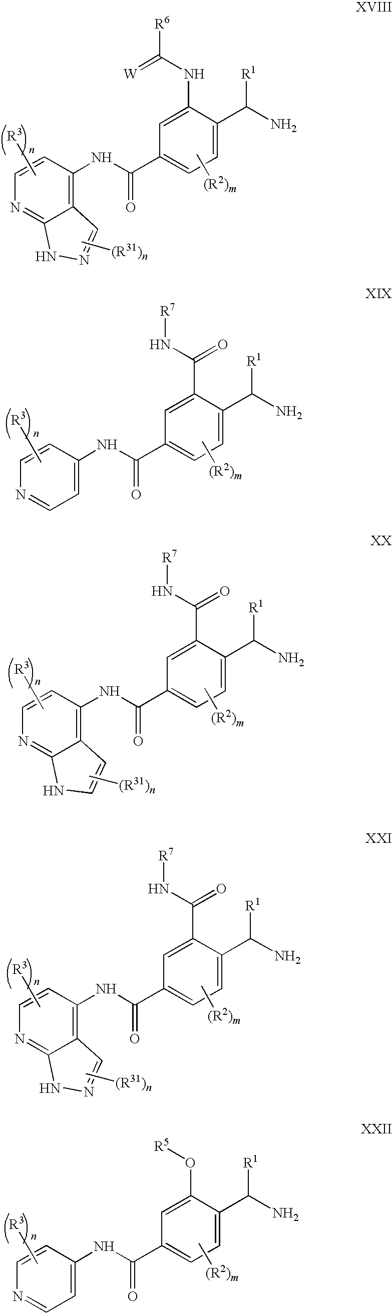 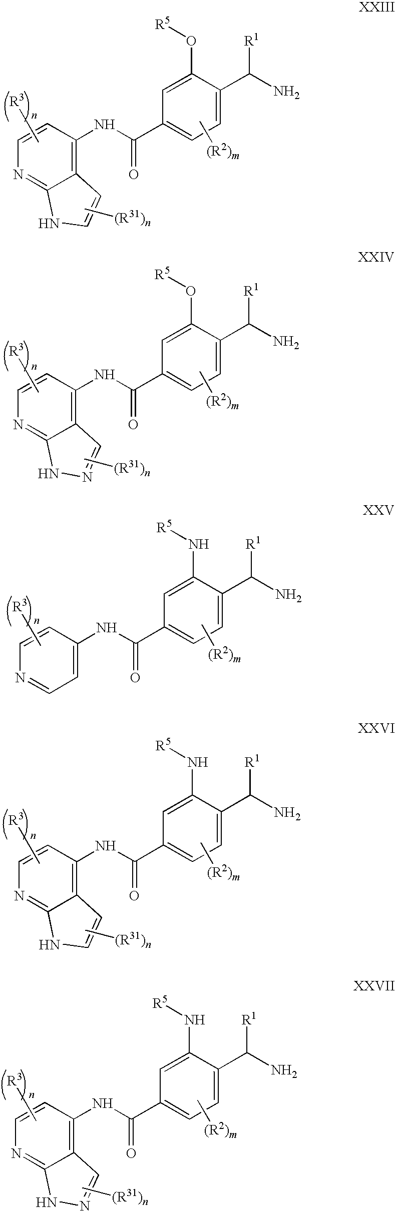
W is selected from O or S,
R⁵ is hydrogen or a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy, and
R¹, R², R³, R³¹, n, m have the same meaning as that defined in any of claims 1 to 3.

6. A compound according to claim 5, wherein:
W is selected from O or S,
R⁵ is hydrogen or a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl, cycloalkyl, heteroaryl, heterocyclyl, each group being optionally substituted by one or more substituents selected from hydroxyl, halogen, oxo, nitro, amino, cyano, alkyl, alkoxy, haloalkyl, or haloalkoxy,
R² is selected from hydrogen, halogen, nitro, cyano, hydroxyl, alkyl, amino, alkoxy, haloalkoxy, aryl, or heteroaryl, m is 0 or 1, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, or 2.

7. A compound according to claim 5, having one of the structural Formula XIII, XIV, XV, XVI, XVII, XVIII, XIX, XX, or XXI, wherein:
W is selected from O or S,
R⁵ is hydrogen or a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl, cycloalkyl, heteroaryl, or heterocyclyl, each group being optionally substituted by one or more substituents selected from hydroxyl, halogen, oxo, nitro, amino, cyano, alkyl, alkoxy, haloalkyl, or haloalkoxy,
m is 0, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, or 2.

8. A compound according to claim 5, having one of the structural Formula XXII, XXIII, XXIV, XXV, XXVI, or XXVII, wherein:
W is selected from O or S,
R⁵ is a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl, cycloalkyl, heteroaryl, heterocyclyl, each group being optionally substituted by one or more substituents selected from hydroxyl, halogen, oxo, nitro, amino, cyano, alkyl, alkoxy, haloalkyl, or haloalkoxy,
R² is selected from hydrogen, halogen, nitro, cyano, hydroxyl, alkyl, amino, alkoxy, haloalkoxy, aryl, or heteroaryl, m is 0 or 1, and
R³ and R³¹ are each independently selected from halogen, hydroxyl, oxo, nitro, amino, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, or alkylthio, wherein R¹⁵ is alkyl or cycloalkyl, and n is 0, 1, or 2.

9. A compound according to claim 2, wherein:
R¹ is a group selected from alkyl or cycloalkyl, each group being optionally substituted by one or two substituents selected from hydroxyl, halogen, oxo, nitro, amino, cyano, alkyl, alkoxy, haloalkyl, or haloalkoxy,
m is 0,
R³ and R³¹ are each independently selected from halogen, hydroxyl, oxo, nitro, cyano, or alkyl, n is 0, 1, or 2,
Z¹, Z², Z³, Z⁴ are each independently selected from CH or N, wherein at least one of Z¹, Z², Z³, or Z⁴ is a N atom,
A¹, A², A³ are each independently selected from CH, N, NH, O, or S, wherein at least one of A¹, A², A³ is a heteroatom selected from N, O, or S,
R⁴ is selected from halogen, hydroxyl, nitro, cyano, amino, alkyl, alkoxy, haloalkyl, or haloalkoxy, p is 0, 1, 2, or 3, or two R⁴ form together an aryl, heteroaryl, or heterocyclyl fused to the aromatic ring to which they are attached, with p being at least 2,
W is selected from O or S,
R⁵ is a group selected from aryl, heteroaryl, aralkyl, or heteroarylalkyl, each group being optionally substituted by one or two substituents selected from halogen, hydroxyl, nitro, cyano, amino, alkyl, alkoxy, haloalkyl, haloalkoxy,
R⁶ is —NH—R⁷ or group selected from aryl, heteroaryl, aralkyl, heteroarylalkyl, arylalkylamino, or heteroarylalkylamino, each group being optionally substituted by one or two substituents selected from halogen, hydroxyl, nitro, cyano, amino, alkyl, alkoxy, aryl, heteroaryl, haloalkyl, or haloalkoxy, and
R⁷ is a group selected from optionally substituted: aryl, heteroaryl, heterocyclyl, aralkyl, or heteroarylalky, each group being optionally substituted by one or two substituents selected from halogen, hydroxyl, nitro, cyano, amino, alkyl, alkoxy, aryl, heteroaryl, haloalkyl, or haloalkoxy.

10. A compound according to claim 1, wherein
W is selected from O or S,
R⁵ is a group selected from alkyl, hydroxyalkyl, aminoalkyl, aryl, heteroaryl, aralkyl, or heteroarylalkyl, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁶ is a group selected from alkyl, amino, —NH—R⁷, alkyl, aryl, heteroaryl, aralkyl, heteroarylalkyl, arylalkylamino, or heteroarylalkylamino, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R⁷ is a group selected from alkyl, aryl, heteroaryl, heterocyclyl, aralkyl, or heteroarylalky, each group being optionally substituted by one or more substituents selected from halogen, hydroxyl, nitro, cyano, amino, alkyl, alkoxy, aryl, heteroaryl, haloalkyl, haloalkoxy, heterocyclyl, or aryloxy,
R¹ is a group selected from alkyl or cycloalkyl, each group being optionally substituted by one or more substituents selected from hydroxyl, halogen, oxo, nitro, amino, cyano, haloalkyl, or haloalkoxy,
m is 0,
R³ and R³¹ are each independently are selected from halogen, hydroxyl, oxo, nitro, cyano, or alkyl, n is 0, 1, or 2,
Z¹, Z², Z³, Z⁴ are each independently selected from CH or N, wherein at least one of Z¹, Z², Z³, or Z⁴ is a N atom, and
A¹, A², A³ are each independently selected from CH, N, NH, O, or S, wherein at least one of A¹, A², A³ is a heteroatom selected from N, O, or S.

11. A compound according to claim 1, selected from 4-(1-Amino-ethyl)-3-hydroxy-N-pyridin-4-yl-benzamide; 4-(1-Amino-ethyl)-3-nitro-N-pyridin-4-yl-benzamide; 3-Amino-4-(1-amino-ethyl)-N-pyridin-4-yl-benzamide; 6-(1-Amino-ethyl)-4′-chloro-biphenyl-3-carboxylic acid pyridin-4-ylamide; 4-(1-Amino-ethyl)-N-pyridin-4-yl-3-thiophen-2-yl-benzamide; 6-(1-Amino-ethyl)-4′-methoxy-biphenyl-3-carboxylic acid pyridin-4-ylamide; 4-(1-Amino-ethyl)-3-furan-3-yl-N-pyridin-4-yl-benzamide; 4-(1-Amino-ethyl)-3-(2-hydroxy-ethoxy)-N-pyridin-4-yl-benzamide; 4-(1-Amino-ethyl)-3-(3-hydroxy-propoxy)-N-pyridin-4-yl-benzamide; 3-(2-Amino-ethoxy)-4-(1-amino-ethyl)-N-pyridin-4-yl-benzamide; 4-(1-Amino-ethyl)-3-(3-amino-propyl)-N-pyridin-4-yl-benzamide; 4-(1-Amino-ethyl)-3-benzyloxy-N-pyridin-4-yl-benzamide; 4-(1-Amino-ethyl)-3-(3-phenyl-thioureido)-N-pyridin-4-yl-benzamide; 4-(1-Amino-ethyl)-3-(3-phenyl-ureido)-N-pyridin-4-yl-benzamide; 4-(1-Amino-ethyl)-3-benzylamino-N-pyridin-4-yl-benzamide; 4-(1-Amino-ethyl)-3-[(furan-2-ylmethyl)-amino]-N-pyridin-4-yl-benzamide; 4-(1-Amino-ethyl)-3-benzoylamino-N-pyridin-4-yl-benzamide; Furan-2-carboxylic acid [2-(1-amino-ethyl)-5-(pyridin-4-ylcarbamoyl)-phenyl]-amide; 4-(1-Amino-ethyl)-3-(3-methoxy-benzoylamino)-N-pyridin-4-yl-benzamide; 4-(1-Amino-ethyl)-3-(3-chloro-benzoylamino)-N-pyridin-4-yl-benzamide; 4-(1-Amino-ethyl)-3-(4-methoxy-benzoylamino)-N-pyridin-4-yl-benzamide; 4-(1-Amino-ethyl)-3-(4-chloro-benzoylamino)-N-pyridin-4-yl-benzamide; 6-(1-aminoethyl)-N-pyridin-4-ylbiphenyl-3-carboxamide; 6-(1-aminoethyl)-3′-chloro-N-pyridin-4-ylbiphenyl-3-carboxamide; 6-(1-aminoethyl)-3′,4′-dichloro-N-pyridin-4-ylbiphenyl-3-carboxamide; 6-(1-aminoethyl)-4′-fluoro-N-pyridin-4-ylbiphenyl-3-carboxamide; 6-(1-aminoethyl)-3′-methoxy-N-pyridin-4-ylbiphenyl-3-carboxamide; 4-(1-aminoethyl)-3-(5,8-dihydronaphthalen-1-yl)-N-pyridin-4-ylbenzamide; 6-(1-aminoethyl)-4′-methoxy-N-pyridin-4-ylbiphenyl-3-carboxamide; 6-(1-aminoethyl)-4′-cyano-N-pyridin-4-ylbiphenyl-3-carboxamide; 6-(1-aminoethyl)-3′-cyano-N-pyridin-4-ylbiphenyl-3-carboxamide; 4-(1-aminoethyl)-3-(2-naphthyl)-N-pyridin-4-ylbenzamide; 6-(1-aminoethyl)-4′-(hydroxymethyl)-N-pyridin-4-ylbiphenyl-3-carboxamide; 6-(1-aminoethyl)-2′-chloro-N-pyridin-4-ylbiphenyl-3-carboxamide; 6-(1-aminoethyl)-2′-methoxy-N-pyridin-4-ylbiphenyl-3-carboxamide; 4-(1-aminoethyl)-N,3-dipyridin-4-ylbenzamide; 4-(1-aminoethyl)-3-pyridin-3-yl-N-pyridin-4-ylbenzamide; 4-(1-aminoethyl)-3-(2-furyl)-N-pyridin-4-ylbenzamide; 4-(1-aminoethyl)-3-(phenylethynyl)-N-pyridin-4-ylbenzamide; 4-(1-aminoethyl)-N-pyridin-4-yl-3-(pyridin-2-ylethynyl)benzamide; 6-(1-aminoethyl)-N-1H-pyrrolo[2,3-b]pyridin-4-ylbiphenyl-3-carboxamide; 6-(1-aminoethyl)-4′-chloro-N-1H-pyrrolo[2,3-b]pyridin-4-ylbiphenyl-3-carboxamide; 6-(1-aminoethyl)-4′-methoxy-N-1H-pyrrolo[2,3-b]pyridin-4-ylbiphenyl-3-carboxamide; or 6-(1-aminoethyl)-3′-chloro-N-1H-pyrrolo[2,3-b]pyridin-4-ylbiphenyl-3-carboxamide.

12. A compound according to claim 1, selected from 6-(1-aminoethyl)-3′,4′-dichloro-N-1H-pyrrolo[2,3-b]pyridin-4-ylbiphenyl-3-carboxamide; 6-(1-aminoethyl)-4′-fluoro-N-1H-pyrrolo[2,3-b]pyridin-4-ylbiphenyl-3-carboxamide; 6-(1-aminoethyl)-3′-methoxy-N-1H-pyrrolo[2,3-b]pyridin-4-ylbiphenyl-3-carboxamide; 4-(1-aminoethyl)-3-(5,8-dihydronaphthalen-1-yl)-N-1H-pyrrolo[2,3-b]-pyridin-4-ylbenzamide; 6-(1-aminoethyl)-4′-cyano-N-1H-pyrrolo[2,3-b]-pyridin-4-ylbiphenyl-3-carboxamide; 6-(1-aminoethyl)-3′-cyano-N-1H-pyrrolo[2,3-b]-pyridin-4-ylbiphenyl-3-carboxamide; 4-(1-aminoethyl)-3-(2-naphthyl)-N-1H-pyrrolo[2,3-b]-pyridin-4-ylbenzamide; 6-(1-aminoethyl)-4′-(hydroxymethyl)-N-1H-pyrrolo[2,3-b]-pyridin-4-ylbiphenyl-3-carboxamide; 6-(1-aminoethyl)-2′-chloro-N-1H-pyrrolo[2,3-b]-pyridin-4-ylbiphenyl-3-carboxamide; 6-(1-aminoethyl)-2′-methoxy-N-1H-pyrrolo[2,3-b]-pyridin-4-ylbiphenyl-3-carboxamide; 4-(1-aminoethyl)-3-pyridin-4-yl-N-1H-pyrrolo[2,3-b]pyridin-4-ylbenzamide; 4-(1-aminoethyl)-3-pyridin-3-yl-N-1H-pyrrolo[2,3-b]pyridin-4-ylbenzamide; 4-(1-aminoethyl)-3-(2-furyl)-N-1H-pyrrolo[2,3-b]pyridin-4-ylbenzamide; 4-(1-aminoethyl)-3-(phenylethynyl)-N-1H-pyrrolo[2,3-b]pyridin-4-ylbenzamide; 4-(1-aminoethyl)-3-(pyridin-2-ylethynyl)-N-1H-pyrrolo[2,3-b]pyridin-4-ylbenzamide; 6-(1-aminoethyl)-4′-hydroxy-N-1H-pyrrolo[2,3-b]pyridin-4-ylbiphenyl-3-carboxamide; 4-(1-aminoethyl)-N-1H-pyrrolo[2,3-b]pyridin-4-yl-3-(2-thienyl)benzamide; 4-(1-aminoethyl)-3-(3-furyl)-N-1H-pyrrolo[2,3-b]pyridin-4-ylbenzamide; 4-(1-aminoethyl)-3-[(anilinocarbonothioyl)amino]-N-1H-pyrrolo[2,3-b]pyridin-4-ylbenzamide; 4-(1-aminoethyl)-3-(benzoylamino)-N-1H-pyrrolo[2,3-b]pyridin-4-ylbenzamide; N-{2-(1-aminoethyl)-5-[(1H-pyrrolo[2,3-b]pyridin-4-ylamino)carbonyl]phenyl}-2-furamide; 6-(1-aminoethyl)-N-1H-pyrazolo[3,4-b]pyridin-4-ylbiphenyl-3-carboxamide; 6-(1-aminoethyl)-4′-chloro-N-1H-pyrazolo[3,4-b]pyridin-4-ylbiphenyl-3-carboxamide; 6-(1-aminoethyl)-4′-methoxy-N-1H-pyrazolo[3,4-b]pyridin-4-ylbiphenyl-3-carboxamide; 6-(1-aminoethyl)-3′-chloro-N-1H-pyrazolo[3,4-b]pyridin-4-ylbiphenyl-3-carboxamide; 6-(1-aminoethyl)-3′,4′-dichloro-N-1H-pyrazolo[3,4-b]pyridin-4-ylbiphenyl-3-carboxamide; 6-(1-aminoethyl)-4′-fluoro-N-1H-pyrazolo[3,4-b]pyridin-4-ylbiphenyl-3-carboxamide; 6-(1-aminoethyl)-3′-methoxy-N-1H-pyrazolo[3,4-b]pyridin-4-ylbiphenyl-3-carboxamide; 4-(1-aminoethyl)-3-(5,8-dihydronaphthalen-1-yl)-N-1H-pyrazolo[3,4-b]-pyridin-4-ylbenzamide; 6-(1-aminoethyl)-4′-cyano-N-1H-pyrazolo[3,4-b]-pyridin-4-ylbiphenyl-3-carboxamide; 6-(1-aminoethyl)-3′-cyano-N-1H-pyrazolo[3,4-b]-pyridin-4-ylbiphenyl-3-carboxamide; 4-(1-aminoethyl)-3-(2-naphthyl)-N-1H-pyrazolo[3,4-b]-pyridin-4-ylbenzamide; 6-(1-aminoethyl)-4′-(hydroxymethyl)-N-1H-pyrazolo[3,4-b]-pyridin-4-ylbiphenyl-3-carboxamide; 6-(1-aminoethyl)-2′-chloro-N-1H-pyrazolo[3,4-b]-pyridin-4-ylbiphenyl-3-carboxamide; 6-(1-aminoethyl)-2′-methoxy-N-1H-pyrazolo[3,4-b]-pyridin-4-ylbiphenyl-3-carboxamide; 4-(1-aminoethyl)-3-pyridin-4-yl-N-1H-pyrazolo[3,4-b]pyridin-4-ylbenzamide; 4-(1-aminoethyl)-3-pyridin-3-yl-N-1H-pyrazolo[3,4-b]pyridin-4-ylbenzamide; 4-(1-aminoethyl)-3-(2-furyl)-N-1H-pyrazolo[3,4-b]pyridin-4-ylbenzamide; 4-(1-aminoethyl)-3-(phenylethynyl)-N-1H-pyrazolo[3,4-b]pyridin-4-ylbenzamide; 4-(1-aminoethyl)-3-(pyridin-2-ylethynyl)-N-1H-pyrazolo[3,4-b]pyridin-4-ylbenzamide; or 6-(1-aminoethyl)-4′-hydroxy-N-1H-pyrazolo[3,4-b]pyridin-4-ylbiphenyl-3-carboxamide.

13. A compound according to claim 1, selected from 4-(1-aminoethyl)-N-1H-pyrazolo[3,4-b]pyridin-4-yl-3-(2-thienyl)benzamide; 4-(1-aminoethyl)-3-(3-furyl)-N-1H-pyrazolo[3,4-b]pyridin-4-ylbenzamide; 4-(1-aminoethyl)-3-[(anilinocarbonothioyl)amino]-N-1H-pyrazolo[3,4-b]pyridin-4-ylbenzamide; 4-(1-aminoethyl)-3-(benzoylamino)-N-1H-pyrazolo[3,4-b]pyridin-4-ylbenzamide; N-{2-(1-aminoethyl)-5-[(1H-pyrazolo[3,4-b]pyridin-4-ylamino)carbonyl]phenyl}-2-furamid; 4-((R)-1-amino-propyl)-N-pyridin-4-yl-3-thiophen-2-yl-benzamide; 4-((S)-1-amino-propyl)-N-pyridin-4-yl-3-thiophen-2-yl-benzamide; 4-((R)-1-amino-propyl)-N-(1H-pyrrolo[2,3-b]pyridin-4-yl)-3-thiophen-2-yl-benzamide; 4-((S)-1-amino-propyl)-N-(1H-pyrrolo[2,3-b]pyridin-4-yl)-3-thiophen-2-yl-benzamide; 4-((R)-amino-cyclopropyl-methyl)-N-pyridin-4-yl-3-thiophen-2-yl-benzamide; 4-((R)-amino-cyclopropyl-methyl)-N-(1H-pyrrolo[2,3-b]pyridin-4-yl)-3-thiophen-2-yl-benzamide; 4-((R)-amino-cyclobutyl-methyl)-N-pyridin-4-yl-3-thiophen-2-yl-benzamide; 4-((R)-amino-cyclobutyl-methyl)-N-(1H-pyrrolo[2,3-b]pyridin-4-yl)-3-thiophen-2-yl-benzamide; 4-((R)-amino-cyclopentyl-methyl)-N-pyridin-4-yl-3-thiophen-2-yl-benzamide; 4-((R)-amino-cyclopentyl-methyl)-N-(1H-pyrrolo[2,3-b]pyridin-4-yl)-3-thiophen-2-yl-benzamide; 4-((R)-1-amino-ethyl)-N-(3-fluoro-pyridin-4-yl)-3-thiophen-2-yl-benzamide; 4-((R)-1-amino-ethyl)-N-(2-fluoro-pyridin-4-yl)-3-thiophen-2-yl-benzamide; 4-((R)-1-Amino-ethyl)-N-pyridin-4-yl-3-thiophen-2-yl-benzamide; or 4-((S)-1-Amino-ethyl)-N-pyridin-4-yl-3-thiophen-2-yl-benzamide.

14. A pharmaceutical and/or veterinary composition comprising a compound as defined in claim 1.

15. A pharmaceutical and/or veterinary composition as claimed in claim 14 comprising at least one carrier, excipient, or diluent acceptable for pharmaceutical and/or veterinary purposes.

16. (canceled)

17. A method for the prevention and/or treatment of at least one disease and/or disorder selected from the group comprising eye diseases; erectile dysfunction; cardiovascular diseases; vascular diseases; inflammatory diseases; proliferative diseases; neurological diseases, and disease of the central nervous system; bronchial asthma; osteoporosis; renal diseases; and AIDS comprising administering to a patient in need of such treatment an effective amount of the compound as defined in claim 1.

18. A method for the prevention and/or treatment of eyes diseases including retinopathy, macular degeneration, and glaucoma, and/or for preventing, treating and/or alleviating complications and/or symptoms associated therewith comprising administering to a patient in need of such treatment an effective amount of the compound as defined in claim 1.

19. A method for the prevention and/or treatment of cardiovascular and vascular diseases selected from the group comprising acute stroke, congestive heart failure, cardiovascular ischemia, heart disease, cardiac remodeling, angina, coronary vasospasm, cerebral vasospasm, pulmonary vasoconstriction, restenosis, hypertension, pulmonary hypertension, arteriosclerosis, thrombosis including deep thrombosis and platelet related diseases, and/or for preventing, treating and/or alleviating complications and/or symptoms associated therewith comprising administering to a patient in need of such treatment an effective amount of the compound as defined in claim 1.

20. A method for the prevention, treatment and/or management of neurological and CNS disorders selected from the group comprising stroke, multiple sclerosis, brain or spinal cord injury, inflammatory, and demyelinating diseases comprising Alzheimer's disease, MS, and neuropathic pain, and/or for preventing, treating and/or alleviating complications and/or symptoms associated therewith comprising administering to a patient in need of such treatment an effective amount of the compound as defined in claim 1.

21. A method for the prevention and/or treatment of cancer selected from the group comprising cancer of the brain (gliomas), breast, colon, intestine, skin, head and neck, kidney, lung, liver, ovarian, pancreatic, prostate, or thyroid; leukemia; lymphoma; sarcoma; melanoma; and/or for preventing, treating and/or alleviating complications and/or symptoms and/or inflammatory responses associated therewith comprising administering to a patient in need of such treatment an effective amount of the compound as defined in claim 1.

22. A method for the prevention and/or treatment of erectile dysfunction, bronchial asthma, osteoporosis, inflammatory diseases, renal diseases, and AIDS, and/or for preventing, treating and/or alleviating complications and/or symptoms associated therewith comprising administering to a patient in need of such treatment an effective amount of the compound as defined in claim 1.

23. A method for the prevention and/or treatment of inflammatory diseases selected from the group comprising contact dermatitis, psoriasis, rheumatoid arthritis, inflammatory bowel disease, Crohn's disease, and ulcerative colitis, and/or for preventing, treating and/or alleviating complications and/or symptoms and/or inflammatory responses associated therewith comprising administering to a patient in need of such treatment an effective amount of the compound as defined in claim 1.

24. A method for inhibiting the activity of at least one kinase, in vitro or in vivo using a compound according to claim 1, or a composition comprising such a compound.

25. The method of claim 24 wherein said use is in vitro.

26. The method of claim 24 wherein the at least one kinase is ROCK.

27. The method according to claim 26 in which the at least one kinase is chosen from the alpha and/or beta isoforms of ROCK.

28. The method according to claim 27 in which the at least one kinase is chosen from the alpha isoform of ROCK.
